(19) United States
(12) Patent Application Publication
(10) Pub. No.: US 2021/0207197 A1
(43) Pub. Date: Jul. 8, 2021

(54) COMPOSITIONS AND METHODS FOR NEXT GENERATION SEQUENCING

(71) Applicant: Twist Bioscience Corporation, South San Francisco, CA (US)

(72) Inventors: Richard GANTT, San Francisco, CA (US); Siyuan CHEN, San Mateo, CA (US)

(21) Appl. No.: 17/154,906

(22) Filed: Jan. 21, 2021

Related U.S. Application Data
(62) Division of application No. 16/798,275, filed on Feb. 21, 2020.
(60) Provisional application No. 62/926,336, filed on Oct. 25, 2019.
(60) Provisional application No. 62/914,904, filed on Oct. 14, 2019.
(60) Provisional application No. 62/810,321, filed on Feb. 25, 2019.

(51) Int. Cl. C12Q 1/6813; C12N 15/10; C12Q 1/6876; C40B 70/00

(52) U.S. Cl. CPC C12Q 1/6813; C12N 15/1086; C12N 2310/3231; C40B 70/00; C12N 2310/15; C12Q 1/6876

(57) ABSTRACT

Provided herein are compositions and methods for next generation sequencing using universal polynucleotide adapters. Further provided are universal adapters using locked nucleic acids or bridged nucleic acids. Further provided are barcoded primers of reduced length for extension of universal adapters. Further provided herein are universal adapter blockers.

### CROSS-REFERENCE

This application is a divisional application of U.S. application Ser. No. 16/798,275, filed Feb. 21, 2020, which claims the benefit of U.S. provisional patent application No. 62/810,321 filed on Feb. 25, 2019, U.S. provisional patent application No. 62/914,904 filed on Oct. 14, 2019, and U.S. provisional patent application No. 62/926,336 filed on Oct. 25, 2019, all of which are incorporated by reference in their entirety.

The instant application contains a Sequence Listing which has been filed electronically in ASCII format and is hereby incorporated by reference in its entirety. Said ASCII copy, created on Mar. 26, 2020, is named 44854-781_401_SL.txt and is 3,235 bytes in size.

### BACKGROUND

Highly efficient chemical gene synthesis with high fidelity and low cost has a central role in biotechnology and medicine, and in basic biomedical research. De novo gene synthesis is a powerful tool for basic biological research and biotechnology applications. While various methods are known for the synthesis of relatively short fragments in a small scale, these techniques often suffer from scalability, automation, speed, accuracy, and cost.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are compositions and methods for next generation sequencing.

Provided herein are polynucleotides, wherein the polynucleotide comprises: a first strand, wherein the first comprises first terminal adapter region, a first non-complementary region, and a first yoke region; a second strand, wherein the second strand comprises comprising a second terminal adapter region, a second non-complementary region, and a second yoke region; wherein the first yoke region and the second yoke region are complementary, wherein the first non-complementary region and the second non-complementary region are not complementary, and wherein the first yoke region or the second yoke region comprises at least one nucleobase analogue. Further provided herein are polynucleotides wherein the nucleobase analogue increases the Tm of binding the first yoke region to the second yoke region. Further provided herein are polynucleotides wherein the nucleobase analogue is a locked nucleic acid (LNA) or a bridged nucleic acid (BNA). Further provided herein are polynucleotides wherein the complementary first yoke region and second yoke region is less than 15 bases in length. Further provided herein are polynucleotides wherein the complementary first yoke region and second yoke region is than 10 bases in length. Further provided herein are polynucleotides wherein the complementary first yoke region and second yoke region is less than 6 bases in length. Further provided herein are polynucleotides wherein the adapter does not comprise a barcode or index sequence.

Provided herein are polynucleotides, wherein the polynucleotide comprises: a duplex sample nucleic acid; a first polynucleotide ligated to a 5′ terminus of the duplex sample nucleic acid; a second polynucleotide ligated to a 3′ terminus of the duplex sample nucleic acid; wherein the first polynucleotide or the second polynucleotide comprises: a first strand comprising a first terminal adapter region, a first non-complementary region, and a first yoke region; and a second strand comprising a second terminal adapter region, a second non-complementary region, and a second yoke region; wherein the first yoke region and the second yoke region are complementary, wherein the first non-complementary region and the second non-complementary region are not complementary, and wherein the first yoke region or the second yoke region comprises at least one nucleobase analogue. Further provided herein are polynucleotides wherein the duplex sample nucleic acid is DNA. Further provided herein are polynucleotides wherein the duplex sample nucleic acid is genomic DNA. Further provided herein are polynucleotides wherein the genomic DNA is of human origin. Further provided herein are polynucleotides wherein the first polynucleotide or the second polynucleotide comprises at least one barcode. Further provided herein are polynucleotides wherein the at least one barcode is at least 8 bases in length. Further provided herein are polynucleotides wherein the at least one barcode is at least 12 bases in length. Further provided herein are polynucleotides wherein the at least one barcode is at least 16 bases in length. Further provided herein are polynucleotides wherein the at least one barcode is 8-12 bases in length. Further provided herein are polynucleotides wherein the first polynucleotide comprises a first barcode and a second barcode, and the second polynucleotide comprises a third barcode and a fourth barcode. Further provided herein are polynucleotides wherein the first barcode and the third barcode have the same sequence, and the second barcode and the fourth barcode have the same sequence. Further provided herein are polynucleotides wherein each barcode in the polynucleotide comprises a unique sequence.

Provided herein are methods of labeling a sample nucleic acid, comprising: (1) ligating at least one polynucleotide to at least one sample nucleic acid to generate an adapter-ligated sample nucleic acid, wherein the polynucleotide comprises: a first strand comprising a first primer binding region, a first non-complementary region, and a first yoke region; and a second strand comprising a second primer binding region, a second non-complementary region, and a second yoke region; wherein the first yoke region and the second yoke region are complementary, and wherein the first non-complementary region and the second non-complementary region are not complementary; (2) contacting at least one adapter-ligated sample nucleic acid with a first primer and a polymerase, wherein the first primer comprises a third primer binding site; a fourth primer binding site; and at least one barcode; wherein the third primer binding site is complementary to less than the length of the at least one polynucleotide adapter, and the third primer binding site is complementary to the first primer binding region; and (3) extending the polynucleotide to generate at least one amplified adapter-ligated sample nucleic acid, wherein the amplified adapter-ligated sample nucleic acid comprises at least one barcode. Further provided herein are methods wherein the primer is less than 30 bases in length. Further provided herein are methods wherein the primer is less than 20 bases in length. Further provided herein are methods wherein the polynucleotide does not comprise a barcode. Further provided herein are methods wherein the primer comprises one barcode. Further provided herein are methods wherein the at least one barcode comprises an index sequence. Further provided herein are methods wherein the at least one barcode is at least 8 bases in length. Further provided herein are methods wherein the at least one barcode is at least 12 bases in length. Further provided herein are methods wherein the at least one barcode is at least 16 bases in length. Further provided herein are polynucleotides wherein the at least one barcode is 8-12 bases in length. Further provided herein are methods wherein the index sequence is common among a library of sample nucleic acids from the same source. Further provided herein are methods wherein the at least one barcode comprises a unique molecular identifier (UMI). Further provided herein are methods wherein two polynucleotides are ligated to sample nucleic acid. Further provided herein are methods wherein a first polynucleotide is ligated to a 5′ terminus of the sample nucleic acid, and a second polynucleotide is ligated to the 3′ terminus of the sample nucleic acid. Further provided herein are methods wherein the method further comprises: (4) contacting at least one adapter-ligated sample nucleic acid with a second primer and a polymerase, wherein the second primer comprises a fifth primer binding site; a sixth primer binding site; and at least one barcode; wherein the sixth primer binding site is complementary to less than the length of the at least one polynucleotide, and the third primer binding site is complementary to the second primer binding region; and (5) extending the polynucleotide to generate at least one amplified adapter-ligated sample nucleic acid, wherein the amplified adapter-ligated sample nucleic acid comprises at least one barcode. Further provided herein are methods further comprising sequencing the adapter-ligated sample nucleic acid.

Provided herein are compositions comprising: at least three polynucleotide blockers, wherein the at least three polynucleotide blockers are configured to bind to one or more regions of an adapter-ligated sample nucleic acid, wherein the adapter-ligated sample nucleic acid comprises: a first non-complementary region, a first index region, a second non-complementary region, and a first yoke region; and a third non-complementary region, a second index region, a fourth non-complementary region, and a second yoke region; wherein the first yoke region and the second yoke region are complementary, and wherein the first non-complementary region and the second non-complementary region are not complementary; and a genomic insert, located adjacent to the first yoke region and the second yoke region, wherein at least one polynucleotide blockers is not complementary to the first yoke region or the second yoke region, and comprises at least one nucleotide analog configured to increase the binding between the polynucleotide blocker and the adapter-ligated sample nucleic acid. Further provided herein are compositions wherein at least two polynucleotide blockers are not complementary to the first yoke region or the second yoke region, and each comprises at least one modified nucleobase configured to increase the binding between the polynucleotide blocker and the adapter-ligated sample nucleic acid. Further provided herein are compositions wherein at least one index region comprises a barcode or unique molecular identifier. Further provided herein are compositions wherein at least one index region is 5-15 bases in length. Further provided herein are compositions wherein at least one of the polynucleotide blockers comprises at least one universal base. Further provided herein are compositions wherein the at least one universal base is 5-nitroindole or 2-deoxyinosine. Further provided herein are compositions wherein the at least one universal base is configured to overlap with at least one index sequence. Further provided herein are compositions wherein at least two universal bases are configured to overlap with at least two index sequences. Further provided herein are compositions wherein at least two of the polynucleotide blockers comprise at least one universal base, wherein each of the at least one universal base overlaps with at least one index sequence. Further provided herein are compositions wherein the overlap is 2-10 bases in length. Further provided herein are compositions wherein the composition comprises no more than four polynucleotide blockers. Further provided herein are compositions wherein the polynucleotide blocker comprises one or more locked nucleic acids (LNAs) or one or more bridged nucleic acids (BNAs). Further provided herein are compositions wherein the polynucleotide blocker comprises at least 5 nucleotide analogues. Further provided herein are compositions wherein the polynucleotide blocker comprises at least 10 nucleotide analogues. Further provided herein are compositions wherein the polynucleotide blocker has a Tm of at least 78 degrees C. Further provided herein are compositions wherein the polynucleotide blocker has a Tm of at least 80 degrees C. Further provided herein are compositions wherein the polynucleotide blocker has a Tm of at least 82 degrees C. Further provided herein are compositions wherein the polynucleotide blocker has a Tm of 80-90 degrees C.

Provided herein are methods for nucleic acid hybridization comprising: providing an adapter-ligated sample nucleic acid library comprising a plurality of genomic inserts; contacting the adapter-ligated sample nucleic acid library with a probe library comprising at least 5000 polynucleotide probes in the presence of the composition of provided herein; and hybridizing at least some of the probes to the genomic inserts. The method of claim 54, wherein the sample nucleic acid library comprises at least 1 million unique genomic inserts. Further provided herein are methods wherein at least some of the genomic inserts comprise human DNA. Further provided herein are methods wherein the method further comprises generating an enriched sample nucleic acid library. Further provided herein are methods wherein the method further comprises sequencing the enriched sample nucleic acid library. Further provided herein are methods wherein the sample nucleic acid library comprises adapters configured for next generation sequencing.

### BRIEF DESCRIPTION OF THE DRAWINGS

FIG. 1A depicts a universal or “stubby” adapter.

FIG. 1B depicts two universal adapters ligated to the ends of a sample nucleic acid.

FIG. 1C depicts a barcoded primer for use in extending universal adapters.

FIG. 1D depicts two universal adapters (after extension/barcode addition) ligated to the ends of a sample polynucleotide.

FIG. 1E depicts barcoded primers binding to universal adapters to generate a barcoded, adapter-ligated sample polynucleotide.

FIG. 1F depicts barcoded primers binding to universal adapters to generate a barcoded, adapter-ligated sample polynucleotide.

FIG. 2 depicts a schematic for ligating barcoded adapters and enriching sample polynucleotides with a probe library prior to sequencing.

FIG. 3 depicts a schematic for ligating universal adapters, adding barcodes to the adapters, and enriching sample polynucleotides with a probe library prior to sequencing.

FIG. 4A depicts concentrations of adapter-ligated sample polynucleotides for standard barcoded Y-adapters or universal adapters.

FIG. 4B depicts AT dropout rates for standard barcoded Y-adapters or universal adapters during whole genome sequencing.

FIG. 5 depicts the number of reads identified for various sample index numbers, wherein the sample indices were added to universal adapters.

FIG. 6A depicts the HS library size for libraries generated using traditional Y adapters with barcodes, universal adapters (with barcodes added by PCR), traditional Y-adapters with UMIs, and universal adapters with UMIs.

FIG. 6B depicts the percent target bases at 30× read depth for libraries generated using traditional Y adapters with barcodes, universal adapters (with barcodes added by PCR), traditional Y-adapters with UMIs, and universal adapters with UMIs.

FIG. 7 depicts capture and enrichment of sample polynucleotides with probes.

FIG. 8 depicts a schematic for generation of polynucleotide libraries from cluster amplification.

FIG. 9A depicts a pair of polynucleotides for targeting and enrichment. The polynucleotides comprise complementary target binding (insert) sequences, as well as primer binding sites.

FIG. 9B depicts a pair of polynucleotides for targeting and enrichment. The polynucleotides comprise complementary target sequence binding (insert) sequences, primer binding sites, and non-target sequences.

FIG. 10A depicts a polynucleotide binding configuration to a target sequence of a larger polynucleotide. The target sequence is shorter than the polynucleotide binding region, and the polynucleotide binding region (or insert sequence) is offset relative to the target sequence, and also binds to a portion of adjacent sequence.

FIG. 10B depicts a polynucleotide binding configuration to a target sequence of a larger polynucleotide. The target sequence length is less than or equal to the polynucleotide binding region, and the polynucleotide binding region is centered with the target sequence, and also binds to a portion of adjacent sequence.

FIG. 10C depicts a polynucleotide binding configuration to a target sequence of a larger polynucleotide. The target sequence is slightly longer than the polynucleotide binding region, and the polynucleotide binding region is centered on the target sequence with a buffer region on each side.

FIG. 10D depicts a polynucleotide binding configuration to a target sequence of a larger polynucleotide. The target sequence is longer than the polynucleotide binding region, and the binding regions of two polynucleotides are overlapped to span the target sequence.

FIG. 10E depicts a polynucleotide binding configuration to a target sequence of a larger polynucleotide. The target sequence is longer than the polynucleotide binding region, and the binding regions of two polynucleotides are overlapped to span the target sequence.

FIG. 10F depicts a polynucleotide binding configuration to a target sequence of a larger polynucleotide. The target sequence is longer than the polynucleotide binding region, and the binding regions of two polynucleotides are not overlapped to span the target sequence, leaving a gap 405.

FIG. 10G depicts a polynucleotide binding configuration to a target sequence of a larger polynucleotide. The target sequence is longer than the polynucleotide binding region, and the binding regions of three polynucleotides are overlapped to span the target sequence.

FIG. 11 presents a diagram of steps demonstrating an exemplary process workflow for gene synthesis as disclosed herein.

FIG. 12 illustrates a computer system.

FIG. 13 is a block diagram illustrating an architecture of a computer system.

FIG. 14 is a diagram demonstrating a network configured to incorporate a plurality of computer systems, a plurality of cell phones and personal data assistants, and Network Attached Storage (NAS).

FIG. 15 is a block diagram of a multiprocessor computer system using a shared virtual address memory space.

FIG. 16 is an image of a plate having 256 clusters, each cluster having 121 loci with polynucleotides extending therefrom.

FIG. 17A is a plot of polynucleotide representation (polynucleotide frequency versus abundance, as measured absorbance) across a plate from synthesis of 29,040 unique polynucleotides from 240 clusters, each cluster having 121 polynucleotides.

FIG. 17B is a plot of measurement of polynucleotide frequency versus abundance absorbance (as measured absorbance) across each individual cluster, with control clusters identified by a box.

FIG. 18 is a plot of measurements of polynucleotide frequency versus abundance (as measured absorbance) across four individual clusters.

FIG. 19A is a plot of on frequency versus error rate across a plate from synthesis of 29,040 unique polynucleotides from 240 clusters, each cluster having 121 polynucleotides.

FIG. 19B is a plot of measurement of polynucleotide error rate versus frequency across each individual cluster, with control clusters identified by a box.

FIG. 20 is a plot of measurements of polynucleotide frequency versus error rate across four clusters.

FIG. 21 is a plot of GC content as a measure of the number of polynucleotides versus percent per polynucleotide.

FIG. 22 depicts a schematic for fragmenting a sample, end repair, A-tailing, ligating universal adapters, and adding barcodes to the adapters via PCR amplification to generate a sequencing library. Additional steps optionally include enrichment, additional rounds of amplification, and/or sequencing (not shown).

FIG. 23 is a plot of the concentration (ng/uL) of ligation products for standard full length Y-adapters amplified by 10 cycles of PCR and universal adapters amplified by 8 cycles of PCR. Universal adapters lead to higher yields of ligation products with fewer PCR cycles.

FIG. 24 shows plots of the concentration of ligation products (measured by fluorescence) vs. ligation product size (bp). The arrows on both graphs indicate the peak corresponding to adapter dimers that do not comprise a genomic polynucleotide insert. Universal adapters (right graph) produce fewer adapter dimers than standard full length Y adapters (left graph).

FIG. 25A is a plot of counts vs. unadjusted, relative sequencing performance for final amplification with universal primers comprising 10 bp dual index sequences or 8 bp dual index sequences (96-plex). Relative sequencing performance was calculated by normalizing the total number of perfect index reads for each design. 10 bp dual index primers exhibited a tighter relative performance and more even sequencing representation.

FIG. 25B is a plot of counts vs. mean centered, relative sequencing performance for final amplification with universal primers comprising 10 bp dual index sequences or 8 bp dual index sequences (96-plex). Relative sequencing performance was calculated by normalizing the total number of perfect index reads for each design and normalizing to the top performer; resulting distributions of each population were centered on their calculated mean for direct comparison. 10 bp dual index primers exhibited a tighter relative performance and more even sequencing representation.

FIG. 26 is a plot of relative barcode performance vs. each barcode sequence for final amplification with universal primers comprising 10 bp dual index sequences or 8 bp dual index sequences (96-plex).

FIG. 27A is a plot of an initial screening set of 1,152 UDI Primer Pairs generated from universal adapters and sequenced as a single pool.

FIG. 27B is a plot of a set of 384 UDI Primer Pairs generated from universal adapters and sequenced as a single pool.

FIG. 27C is a plot of an individual pool of 96 UDI Primer Pairs generated from universal adapters and sequenced independently.

FIG. 27D is a plot of an individual pool of 96 UDI Primer Pairs generated from universal adapters and sequenced independently.

FIG. 27E is a plot of an individual pool of 96 UDI Primer Pairs generated from universal adapters and sequenced independently.

FIG. 27F is a plot of an individual pool of 96 UDI Primer Pairs generated from universal adapters and sequenced independently.

FIG. 28A depicts a plot of uniform coverage (top panel) and non-uniform coverage (bottom panel).

FIG. 28B is a graph of fold 80 base penalty of various comparator panels (Comparator A1, Comparator A2, and Comparator D) and Library 4A.

FIG. 28C depicts a schematic for on-target rate, near-target rate, and off-target rate.

FIG. 28D is a graph of on-target rate of various comparator panels (Comparator A1, Comparator A2, and Comparator D) and Library 4A.

FIGS. 28E-28F depict graphs of duplication rate of various comparator panels (Comparator A1, Comparator A2, and Comparator D) and Library 4A. FIG. 28E depicts HS_library_size, and FIG. 28F depicts a percentage of the fraction of aligned bases that were filtered out because they were in reads marked as duplicates.

FIG. 29 is a graph of depth coverage of various comparator panels (Comparator A1, Comparator A2, and Comparator D) and Library 4A.

FIG. 30A is a first schematic of adding or enhancing content to custom panels.

FIG. 30B is a second schematic of adding or enhancing content to custom panels.

FIG. 30C is a graph of uniformity (fold-80) comparing a panel with and without supplemental probes.

FIG. 30D is a graph of duplicate rate comparing a panel with and without supplemental probes.

FIG. 30E is a graph of percent on rate comparing a panel with and without supplemental probes.

FIG. 30F is a graph of percent target coverage comparing a panel with and without supplemental probes, and comparator enrichment kits.

FIG. 30G is a graph of 80-fold base penalty comparing a panel with and without supplemental probes, and comparator enrichment kits.

FIG. 30H depicts graphs of tunable target coverage of panels.

FIG. 31A is a schematic of the RefSeq design.

FIGS. 31B-31C depict graphs of depth coverage as percent target bases at coverage of the exome panel alone or with the RefSeq panel added. FIG. 31B depicts a first experiment, and FIG. 31C depicts a second experiment.

FIGS. 31D-31H depict graphs of various enrichment/capture sequencing metrics for a standard exome panel vs. the exome panel combined with the RefSeq panel in both singleplex and 8-plex experiments. FIG. 31D shows a graph of specificity as percent off target for the exome panel alone or with the RefSeq panel added. FIG. 31E shows a graph of uniformity for the exome panel alone or with the RefSeq panel added. FIG. 31F shows a graph of library size for the exome panel alone or with the RefSeq panel added. FIG. 31G shows a graph of duplicate rate for the exome panel alone or with the RefSeq panel added. FIG. 31H shows a graph of coverage rate for the exome panel alone or with the RefSeq panel added.

FIG. 32A is a graph of percentage of reads in each custom panel achieving 30× coverage.

FIG. 32B is a graph of the fraction of target bases >30× for each custom panel.

FIG. 32C is a graph of uniformity (fold-80) of each custom panel.

FIG. 33A is a schematic of a fast enrichment workflow.

FIG. 33B depict performance as percent target bases at coverage using the fast hybridization and wash workflow and the hybridization and wash workflow.

FIG. 34A is a graph of percentage of bases on target using nanoball sequencing.

FIG. 34B is a graph of uniformity using nanoball sequencing.

FIG. 34C is a graph of duplication rate using nanoball sequencing.

FIG. 34D is a graph of target bases at 30× coverage or higher.

FIGS. 35A-35E depicts a single molecule of a Next Generation Sequencing library following polymerase chain amplification as thick bars with 5′ & 3′ ends of the ‘top’ and ‘bottom’ strands labelled for orientation. The legend for FIGS. 35A-35E is depicted in FIG. 35A. Blockers with various chemical modifications and/or design features are depicted as thinner blockers with 5′ & 3′ ends labelled for orientation and positioned nearest to the adapter region for which they are designed to bind. FIG. 35A depicts a binding configuration for a set of blockers (‘D’, ‘J’, ‘L’, and ‘E’) that binds all adapter regions interior to the index with a single molecule (‘J’ and ‘L’). FIG. 35B depicts a binding configuration for a set of blockers (‘D’, ‘M’, ‘N’, ‘Q’, and ‘E’) that binds the adapter region interior to the index with multiple blockers. Note that the Y-stem annealing portion of the adapters is bound with a single blocker member ‘N’. FIG. 35C depicts an alternative binding configuration for a set of blockers (‘D’, ‘M’, ‘P’, ‘Q’, and ‘E’) that binds the adapter region interior to the index with multiple blockers. Note that the Y-stem annealing portion of the adapters is bound with a single blocker member ‘P’. FIG. 35D depicts a binding configuration for a set of blockers (‘R’, ‘N’, and ‘S’) that binds the adapter region interior to the index with multiple blockers. In this case the binding of adapter sequences exterior to the index, the adapter index, and interior to the index interact with a single unique molecule on each side. Note that the Y-stem annealing portion of the adapters is bound with a single blocker member ‘N’. Note that only a single adapter index length is addressable with such a binding configuration. FIG. 35E depicts an alternative binding configuration for a set of blockers that binds the adapter region interior to the index with multiple blockers. In this case the binding of adapter sequences exterior to the index, the adapter index, and interior to the index interact with a single unique molecule on each side. Note that the Y-stem annealing portion of the adapters is bound with a single blocker member ‘P’. Note that only a single adapter index length is addressable with such a binding configuration.

FIGS. 36A-36D depicts a single molecule of a Next Generation Sequencing library following polymerase chain amplification as thick bars with 5′ & 3′ ends of the ‘top’ and ‘bottom’ strands labelled for orientation. The legend for FIGS. 36A-36D is depicted in FIG. 36A. Blockers with various chemical modifications and/or design features are depicted as thinner blockers with 5′ & 3′ ends labelled for orientation and positioned nearest to the adapter region for which they are designed to bind. FIG. 36A depicts all blockers binding in a desired configuration. This is a desired population that leads to optimal performance of target enrichment workflows. FIG. 36B depicts exterior blockers binding in the desired configuration. This is an undesired population. Interior blockers binding in an undesired configuration with unbound regions that can recruit other molecules that include adapter sequences on other molecules that are not desired. FIG. 36C depicts blockers binding to each other in solution. This is an undesired population. Blockers bind to each other and cannot bind to their designated adapter regions. FIG. 36D depicts blockers free in solution. This is a neutral population that has minimal effect on performance of target enrichment workflows.

FIGS. 37A-37G depicts a single molecule of a Next Generation Sequencing library following polymerase chain amplification as thick bars with 5′ & 3′ ends of the ‘top’ and ‘bottom’ strands labelled for orientation. The legend for FIGS. 37A-37G is depicted in FIG. 37A. Blockers with various chemical modifications and/or design features are depicted as thinner blockers with 5′ & 3′ ends labelled for orientation and positioned nearest to the adapter region for which they are designed to bind. FIG. 37A depicts a set of blockers designed for (1) dual index adapters where (2) all blockers bind to a single strand, (3) blockers designed to bind region exterior to index are not extended to cover adapter index, and (4) blockers designed to bind adapter region interior to index are not extended to cover adapter index. FIG. 37B depicts a set of blockers designed for (1) dual index adapters where (2) all blockers bind to a single strand, (3) blockers designed to bind region exterior to index are extended to cover adapter index, and (4) blockers designed to bind adapter region interior to index are not extended to cover adapter index. FIG. 37C depicts a set of blockers designed for (1) dual index adapters where (2) all blockers bind to a single strand, (3) blockers designed to bind region exterior to index are not extended to cover adapter index, and (4) blockers designed to bind adapter region interior to index are extended to cover adapter index. FIG. 37D depicts a set of blockers designed for (1) dual index adapters where (2) all blockers bind to a single strand, (3) blockers designed to bind region exterior to index are extended to cover adapter index, and (4) blockers designed to bind adapter region interior to index are extended to cover adapter index. FIG. 37E depicts a set of blockers designed for (1) dual index adapters where (2) blockers bind to a both strands, (3) blockers designed to bind region exterior to index are extended to cover adapter index, and (4) blockers designed to bind adapter region interior to index are extended to cover adapter index. FIG. 37F depicts a set of blockers designed for (1) single index adapters where (2) all blockers bind to a single strand, (3) blockers designed to bind region exterior to index are extended to cover adapter index (if present), and (4) blockers designed to bind adapter region interior to index are extended to cover adapter index (if present). FIG. 37G depicts a set of blockers designed for (1) dual index adapters where (2) all blockers bind to a single strand, (3) blockers designed to bind region exterior to index are extended to cover adapter index, (4) blockers designed to bind adapter region interior to index are extended to cover adapter index, and (5) blockers designed to bind adapter region interior to index are extended to cover unique molecular identifier index (or other polynucleotide sequence that could be defined or undefined).

FIG. 38 depicts a graph of performance of blocker sets that cover various number of index bases as a function of percent off bait.

FIGS. 39A-39C depicts one strand of a single molecule of a Next Generation Sequencing library following polymerase chain amplification is depicted as thick bars with 5′ & 3′ ends of the ‘top’ and ‘bottom’ strands labelled for orientation. The legend for FIGS. 39A-39C is depicted in FIG. 39A. Blockers with various chemical modifications and/or design features are depicted as thinner blockers with 5′ & 3′ ends labelled for orientation and positioned nearest to the adapter region for which they are designed to bind. Here different binding modes for two blockers designed to cover three adapter index bases from both sides are shown in different binding modes for adapters. FIG. 39A depicts a 6 bp adapter index length, 6 total index bases covered by an overhang, 0 total index bases exposed resulting in 0% total index bases exposed. FIG. 39B depicts a 8 bp adapter index length, 6 total index bases covered by an overhang, 2 total index bases exposed resulting in 25% total index bases exposed.

FIG. 39C depicts a 10 bp adapter index length, 6 total index bases covered by an overhang, 4 total index bases exposed resulting in 40% total index bases exposed.

FIGS. 40A-40L depicts one strand of a single molecule of a Next Generation Sequencing library following polymerase chain amplification is depicted as thick bars with 5′ & 3′ ends of the ‘top’ and ‘bottom’ strands labelled for orientation. The legend for FIGS. 40A-40L is depicted in FIG. 40A. Blockers with various chemical modifications and/or design features are depicted as thinner blockers with 5′ & 3′ ends labelled for orientation and positioned nearest to the adapter region for which they are designed to bind. FIG. 40A depicts blockers for a (1) dual index system designed to (2) bind to a single strand with (3) no modification for binding to Y-stem annealing portion of adapters and (4) extension to cover adapter index. FIG. 40B depicts blockers for a (1) dual index system designed to (2) bind to both strands with (3) no modification for binding to Y-stem annealing portion of adapters and (4) extension to cover adapter index. FIG. 40C depicts blockers for a (1) single index system designed to (2) bind to a single strand with (3) no modification for binding to Y-stem annealing portion of adapters and (4) extension to cover adapter index. FIG. 40D depicts blockers for a (1) dual index system designed to (2) bind to a single strand with (3) no modification for binding to Y-stem annealing portion of adapters, (4) extension to cover adapter index, and (5) extension to cover unique molecular identifier index. FIG. 40E depicts blockers for a (1) dual index system designed to (2) bind to a single strand with (3) modification to decrease binding affinity to Y-stem annealing portion of adapters and (4) extension to cover adapter index. FIG. 40F depicts blockers for a (1) dual index system designed to (2) bind to both strands with (3) modification to decrease binding affinity to Y-stem annealing portion of adapters and (4) extension to cover adapter index. FIG. 40G depicts blockers for a (1) single index system designed to (2) bind to a single strand with (3) modification to decrease binding affinity to Y-stem annealing portion of adapters and (4) extension to cover adapter index. FIG. 40H depicts blockers for a (1) dual index system designed to (2) bind to a single strand with (3) modification to decrease binding affinity to Y-stem annealing portion of adapters, (4) extension to cover adapter index, and (5) extension to cover unique molecular identifier index. FIG. 40I depicts blockers for a (1) dual index system designed to (2) bind to a single strand with (3) a single member to bind to Y-stem annealing portion of adapters and (4) extension to cover adapter index. FIG. 40J depicts blockers for a (1) dual index system designed to (2) bind to both strands with (3) a single member to bind to Y-stem annealing portion of adapters and (4) extension to cover adapter index. FIG. 40K depicts blockers for a (1) single index system designed to (2) bind to a single strand with (3) a single member to bind to Y-stem annealing portion of adapters and (4) extension to cover adapter index. FIG. 40L depicts blockers for a (1) dual index system designed to (2) bind to a single strand with (3) a single member to bind to Y-stem annealing portion of adapters, (4) extension to cover adapter index, and (5) extension to cover unique molecular identifier index.

FIG. 41 depicts a workflow for unmethylated samples (top) and methylated samples (bottom).

FIGS. 42A-42D depict graphs of sequencing metrics for three different sizes of standard methylated panels. FIG. 42A depicts a graph of the percentage of bases at 30× coverage. FIG. 42B depicts a graph of the fold-80 base penalty. FIG. 42C depicts a graph of percent off bait. FIG. 42D depicts a graph of the duplication rate.

FIGS. 43A-43D depicts graphs of sequencing metrics for an optimized 1 Mb methylated panel with high, medium, or low stringency. FIG. 43A depicts a graph of the percentage of bases at 30× coverage. FIG. 43B depicts a graph of the fold-80 base penalty. FIG. 43C depicts a graph of percent off bait. FIG. 43D depicts a graph of the duplication rate.

FIGS. 44A-44D depicts graphs of sequencing metrics for an optimized 1 Mb methylated panel of medium stringency used to capture targets from gDNA libraries generated from hypomethylated and hypermethylated cell lines blended to final ratios of 0, 25, 50, 75, and 100% methylation. FIG. 44A depicts a graph of the percentage of bases at 30× coverage. FIG. 44B depicts a graph of the fold-80 base penalty. FIG. 44C depicts a graph of percent off bait. FIG. 44D depicts a graph of the duplication rate.

FIGS. 45A-45B depict the detection of different DNA methylation levels along targets and individual CpG sites in the clinically relevant Cyclin D2 locus, which is known to change methylation states in certain cancers (e.g., breast cancer). FIG. 45A depicts methylation at the genomic locus from 4,268 kb to 4,276 kb. FIG. 45B depicts methylation at the genomic locus from 4,275.2 kb to 4,276.4 kb.

FIGS. 46A-46D depict graphs of sequencing metrics for an optimized 1 Mb methylated panel of medium stringency used to capture targets using either bisulfite or enzymatic conversion methods. FIG. 46A depicts a graph of the percentage of bases at 30× coverage. FIG. 46B depicts a graph of the fold-80 base penalty. FIG. 46C depicts a graph of percent off bait. FIG. 46D depicts a graph of the duplication rate.

FIG. 47 depicts a box graph of conversion rates, measured as the fraction of cytosines converted in non-CpG sites were >99.5% for both bisulfite and enzymatic conversion methods.

### DETAILED DESCRIPTION

Described herein are composition and methods for next generation sequencing, including polynucleotide adapters and hybridization blockers. Traditional adapters often comprise barcode regions that comprise information related to sample index/origin, or unique molecular identifiers; such barcodes are ligated directly to sample nucleic acids. However, in some cases a requirement for high purity and significant synthetic overhead in producing barcoded adapters limits their performance in next generation sequencing applications. Alternatively, truncated “universal” (or stubby) adapters without barcodes are ligated to sample nucleic acids, and libraries of barcodes are added at a later stage before sequencing. Such universal adapters in some instances are cheaper to produce, and provide higher ligation efficiencies than traditional barcoded adapters. Higher ligation efficiencies in some instances allow fewer PCR cycles for amplification, which leads to lower PCR-induced amplification errors. In some instances, barcode libraries that are added to universal adapters comprise a higher number of barcodes, or barcodes that are longer than typical barcoded adapters. Additionally, universal adapters are compatible with a wide range of different sequencing platforms. Further provided herein are universal adapters comprising nucleobase analogues. Further provided herein are barcoded primers, wherein the length of a universal adapter binding region of the primer is less than the length of the universal adapter. Described herein are hybridization blockers prevent unwanted adapter-adapter interactions to increase enrichment efficiency metrics. Further described herein are hybridization blockers with various adapter-binding configurations. Further described herein are methods of identifying methylation modifications to genomic DNA.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

The term nucleic acid encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. The length of polynucleotides, when provided, are described as the number of bases and abbreviated, such as nt (nucleotides), bp (bases), kb (kilobases), Mb (megabases) or Gb (gigabases).

Provided herein are methods and compositions for production of synthetic (i.e. de novo synthesized or chemically synthesizes) polynucleotides. The term oligonucleic acid, oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria) and eukaryotes (e.g., mice, rabbits, humans, and non-human primates). In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or to 50,000 to 1,000,000 polynucleotides. A polynucleotide library described herein may comprise about 370,000; 400,000; 500,000 or more different polynucleotides.

Universal Adapters

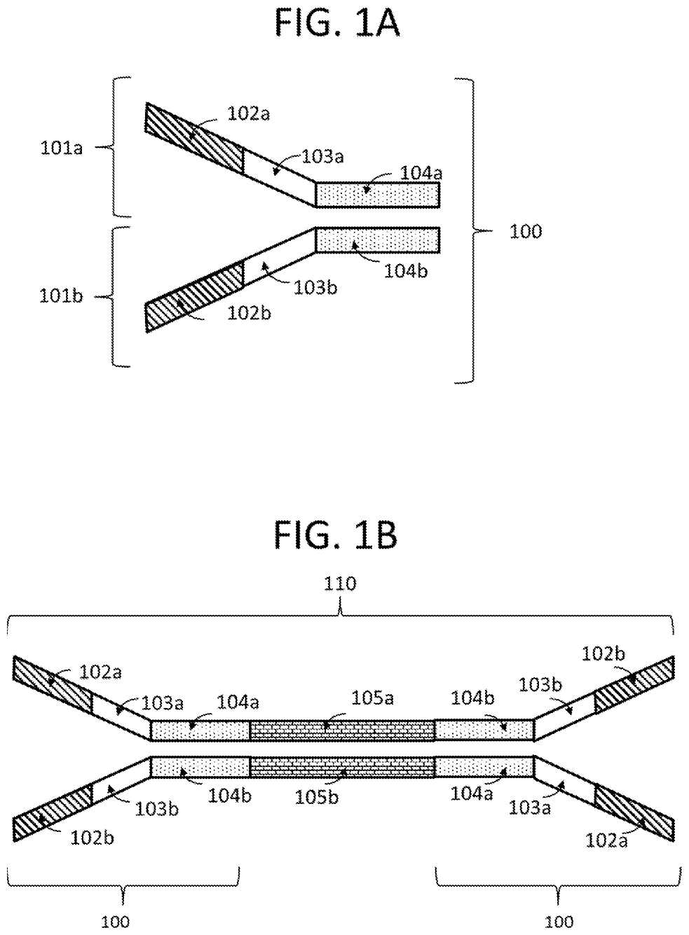

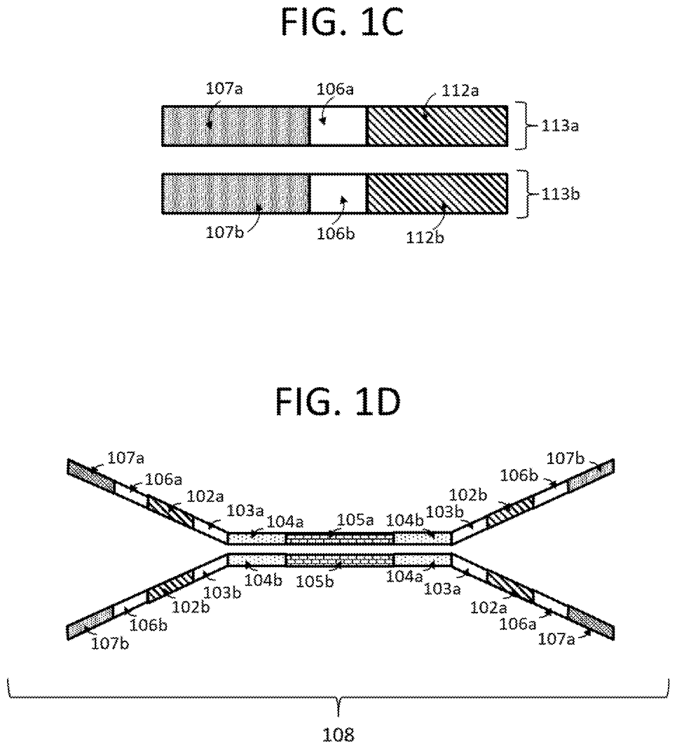

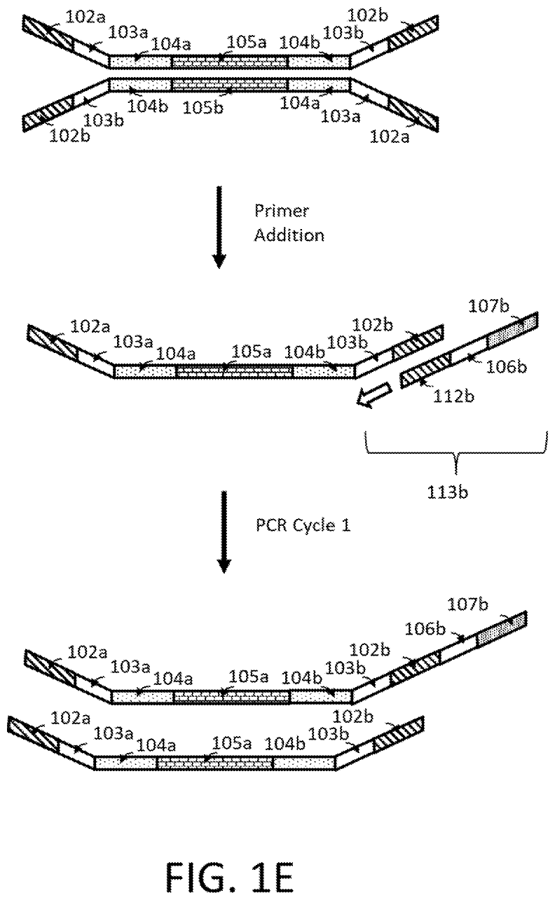

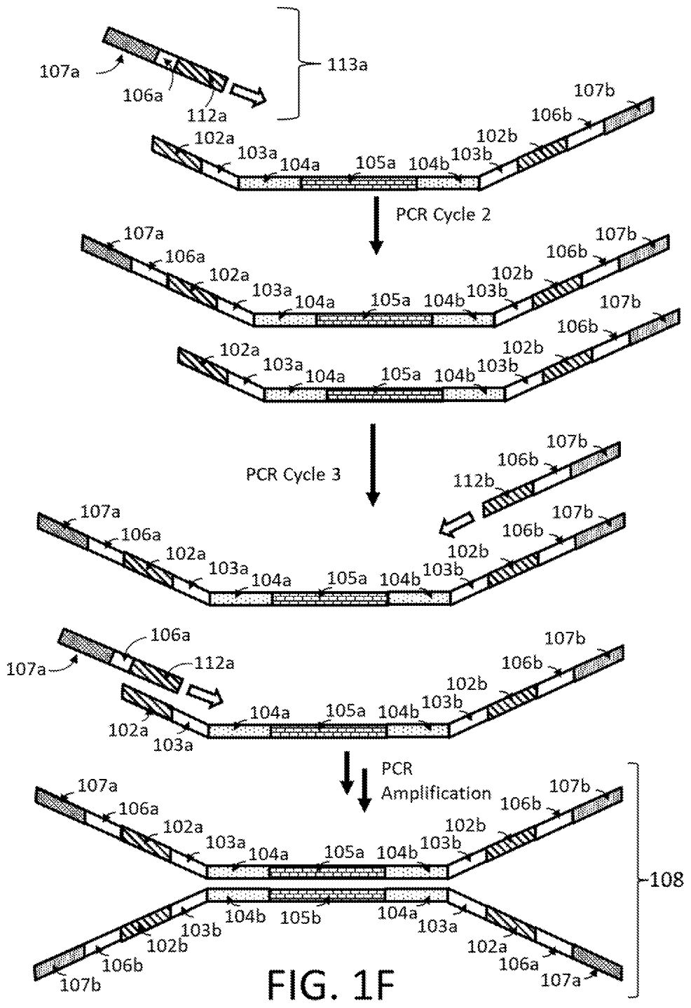

As depicted in FIG. 1A, in some instances, the universal adapters disclosed herein may comprise a universal polynucleotide adapter 100 comprising a first strand 101a and a second strand 101b. In some instances, a first strand 101a comprises a first primer binding region 102a, a first non-complementary region 103a, and a first yoke region 104a. In some instances, a second strand 101b comprises a second primer binding region 102b, a second non-complementary region 103b, and a second yoke region 104b. In some instances, a primer (e.g., 102a/102b) binding region allows for PCR amplification of a polynucleotide adapter 100. In some instances, a primer (e.g., 102a/102b) binding region allows for PCR amplification of a polynucleotide adapter 100 and concurrent addition of one or more barcodes to the polynucleotide adapter. In some instances, the first yoke region 104a is complementary to the second yoke region 104b. In some instances, the first non-complementary region 103a is not complementary to the second non-complementary region 103b. In some instances, the universal adapter 100 is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier.

A universal (polynucleotide) adapter 100 may be shortened relative to a typical barcoded adapter (e.g., full-length “Y adapter”). For example, a universal adapter strand 101a or 101b is 20-45 bases in length. In some instances, a universal adapter strand is 25-40 bases in length. In some instances, a universal adapter strand is 30-35 bases in length. In some instances, a universal adapter strand is no more than 50 bases in length, no more than 45 bases in length, no more than 40 bases in length, no more than 35 bases in length, no more than 30 bases in length, or no more than 25 bases in length. In some instances, a universal adapter strand is about 25, 27, 30, 32, 34, 36, 38, 40, 42, 44, 46, 48, 50, 52, 54, 56, 58, or about 60 bases in length. In some instances, a universal adapter strand is about 60 base pairs in length. In some instances, a universal adapter strand is about 58 base pairs in length. In some instances, a universal adapter strand is about 52 base pairs in length. In some instances, a universal adapter strand is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. As depicted in FIG. 1B, an adapter-ligated sample polynucleotide 110 in some instances comprises a sample polynucleotide (e.g., sample nucleic acid) (105a/105b) with adapters 100 ligated to both the 5′ and 3′ end of the sample polynucleotide 105a/105b. A duplex sample polynucleotide comprises both a first strand (forward) 105a and a second strand (reverse) 105b.

Universal adapters may contain any number of different nucleobases (DNA, RNA, etc.), nucleobase analogues, or non-nucleobase linkers or spacers. For example, an adapter comprises one or more nucleobase analogues or other groups that enhance hybridization (Tₘ) between two strands of the adapter. In some instances, nucleobase analogues are present in the yoke region of an adapter. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps. In some instances, adapters comprise one or more nucleobase analogues selected from Table 1.

TABLE 1

| Base | A | T | G |
|---|---|---|---|
| Lock- ed Nu- cleic Acid (LNA) | 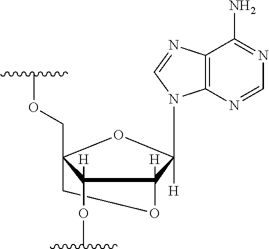 [SMILES: *OC[C@@]12COC([C@H](n3cnc4c(N)ncnc43)O1)[C@H]2O*] | 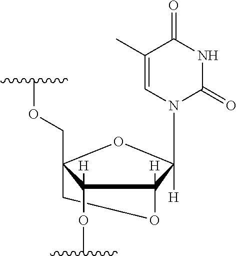 [SMILES: *OC[C@@]12COC([C@H](n3cc(C)c(=O)[nH]c3=O)O1)[C@H]2O*] | 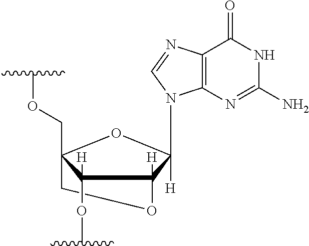 [SMILES: *OC[C@@]12COC([C@H](n3cnc4c(=O)[nH]c(N)nc43)O1)[C@H]2O*] |
| Bridg- ed Nu- cleic Acid* (BNA) | 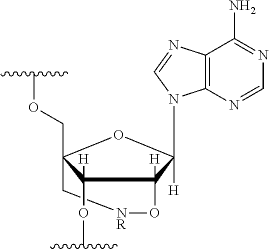 | 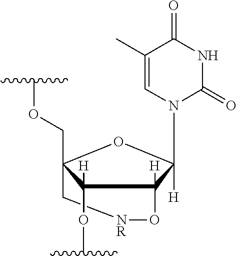 | 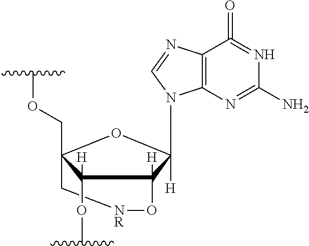 |
| | Base | C | U |
| | Locked Nucleic Acid (LNA) | 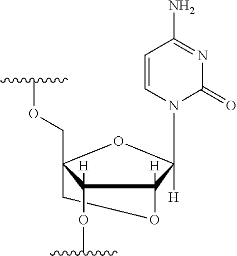 [SMILES: *OC[C@@]12COC([C@H](n3ccc(N)nc3=O)O1)[C@H]2O*] | 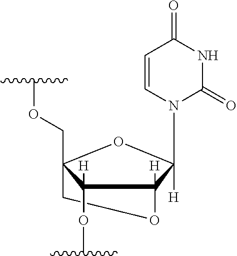 [SMILES: *OC[C@@]12COC([C@H](n3ccc(=O)[nH]c3=O)O1)[C@H]2O*] |
| | Bridged Nucleic Acid* (BNA) | 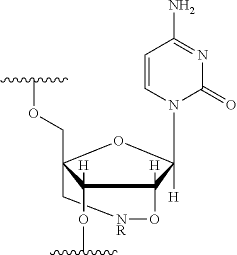 | 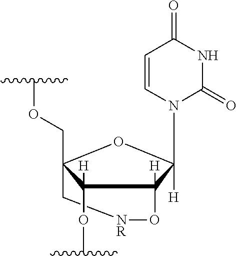 |

*R is H or Me.

Universal adapters may comprise any number of nucleobase analogues (such as LNAs or BNAs), depending on the desired hybridization Tₘ. For example, an adapter comprises 1 to 20 nucleobase analogues. In some instances, an adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, adapters (e.g., universal adapters) described herein comprise methylated nucleobases, such as methylated cytosine.

### Barcoded Primers

Polynucleotide primers may comprise defined sequences, such as barcodes (or indices), as depicted in FIG. 1C. Barcodes can be attached to universal adapters, for example, using PCR and barcoded primers 113a or 113b to generate barcode, adapter-ligated sample polynucleotides FIG. 1D, 108. Primer binding sites, such as universal primer binding sites 107a or 107b depicted in FIGS. 1C and 1D, facilitate simultaneous amplification of all members of a barcode primer library, or a subpopulation of members. In some instances, a primer binding site 107a or 107b comprises a region that binds to a flow cell or other solid support during next generation sequencing. In some instances, a barcoded primer comprises a P5 (5′-AATGATACGGCGACCACCGA-3′ (SEQ ID NO: 5)) or P7 (5′-CAAGCAGAAGACGGCATACGAGAT-3′ (SEQ ID NO: 6)) sequence. In some instances, primer binding sites 112a or 112b are configured to bind to universal adapter sequences 102a or 102b, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

Barcoded primers comprise one or more barcodes 106a or 106b, as depicted in FIGS. 1C and 1D. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. Barcoded primers or adapters may comprise unique molecular identifiers (UMI). Such UMIs in some instances uniquely tag all nucleic acids in a sample. In some instances, at least 60%, 70%, 80%, 90%, 95%, or more than 95% of the nucleic acids in a sample are tagged with a UMLI. In some instances, at least 85%, 90%, 95%, 97%, or at least 99% of the nucleic acids in a sample are tagged with a unique barcode, or UMLI. Barcoded primers in some instances comprise an index sequence and one or more UMLI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode.

Barcoded primers in a library comprise a region that is complementary 112a/112b to a primer binding region 102a/102b on a universal adapter, as depicted in FIGS. 1E and 1F. For example, universal adapter binding region 112a is complementary to primer region 102a of the universal adapter, and universal adapter binding region 112b is complementary to primer region 102b of the universal adapter. Such arrangements facilitate extension of universal adapters during PCR, and attach barcoded primers (as depicted in FIGS. 1E and 1F). In some instances, the Tm between the primer and the primer binding region is 40-65 degrees C. In some instances, the Tm between the primer and the primer binding region is 42-63 degrees C. In some instances, the Tm between the primer and the primer binding region is 50-60 degrees C. In some instances, the Tm between the primer and the primer binding region is 53-62 degrees C. In some instances, the Tm between the primer and the primer binding region is 54-58 degrees C. In some instances, the Tm between the primer and the primer binding region is 40-57 degrees C. In some instances, the Tm between the primer and the primer binding region is 40-50 degrees C. In some instances, the Tm between the primer and the primer binding region is about 40, 45, 47, 50, 52, 53, 55, 57, 59, 61, or 62 degrees C.

Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances be described as a “set”, wherein the set comprises In some instances, universal blockers prevent adapter-adapter interactions independent of one or more barcodes present on at least one of the adapters. For example, a blocker comprises one or more nucleobase analogues or other groups that enhance hybridization (Tₘ) between the blocker and the adapter. In some instances, a blocker comprises one or more nucleobases which decrease hybridization (Tₘ) between the blocker and the adapter (e.g., “universal” bases). In some instances, a blocker described herein comprises both one or more nucleobases which increase hybridization (Tₘ) between the blocker and the adapter and one or more nucleobases which decrease hybridization (Tₘ) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

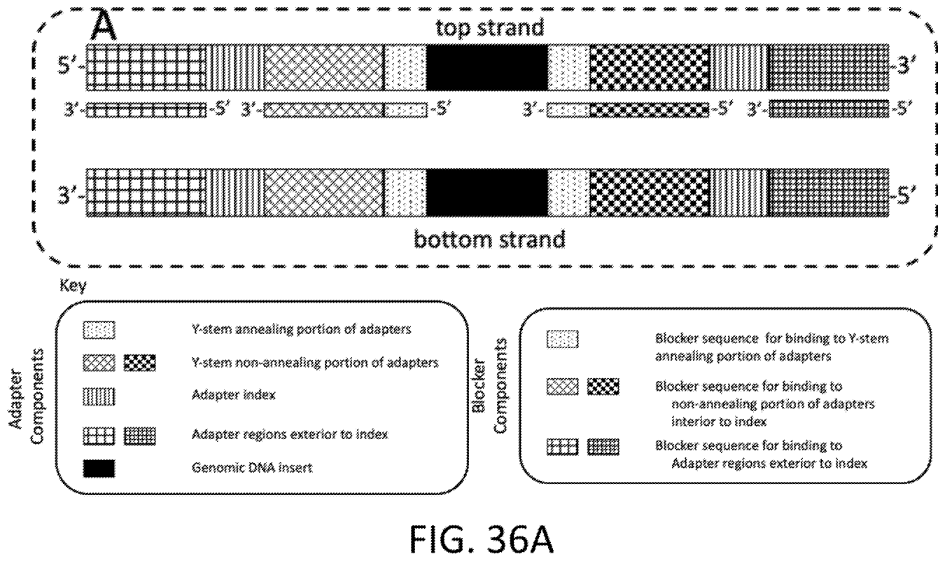

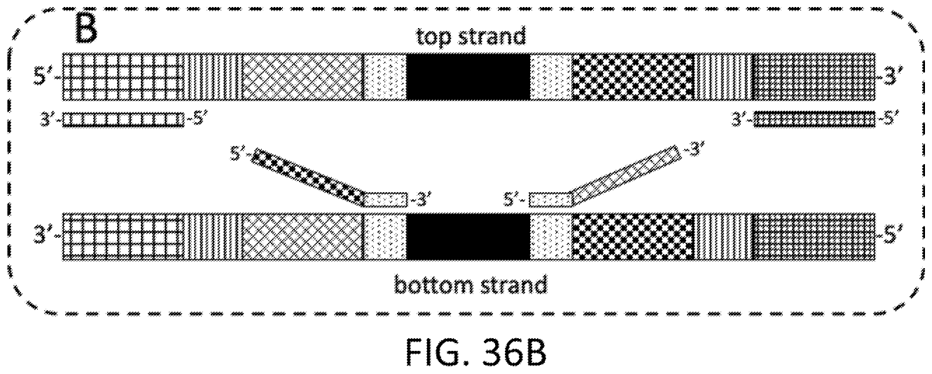

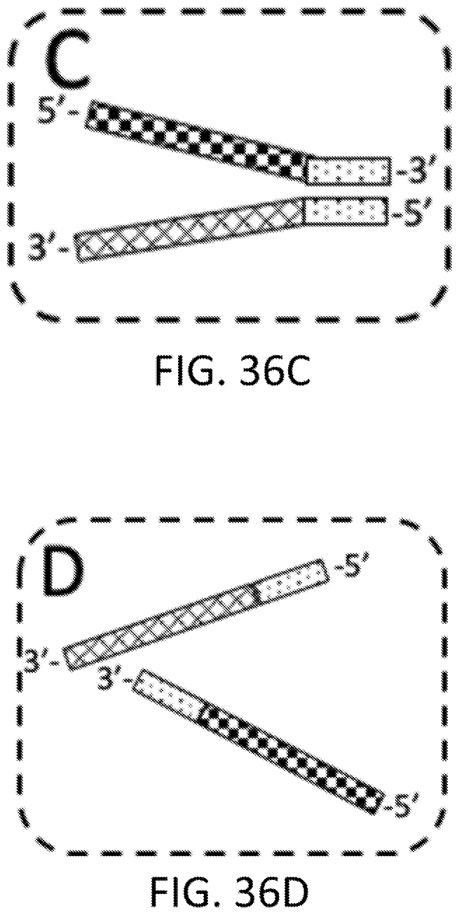

Blockers (e.g., universal blockers) and adapters may form a number of different populations during hybridization. In some instances, when the number of DNA modifications that decrease affinity in the Y-stem annealing region of the blocker are increased, the populations ‘A’ & ‘D’ dominate and either have the desired (A, FIG. 36A) or minimal effect (D, FIG. 36D). In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’ FIG. 36B) or sequester blockers where they are unable to function properly (C, FIG. 36C).

The index on both single or dual index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

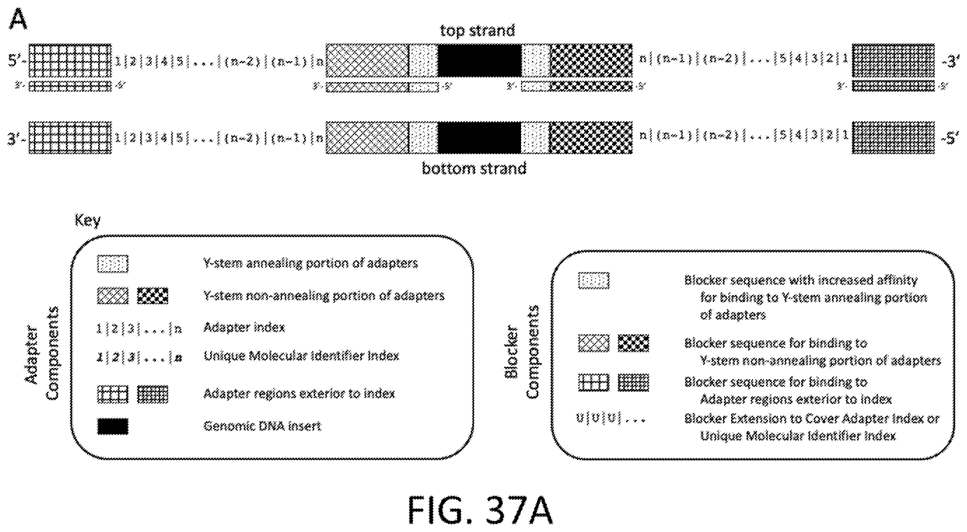

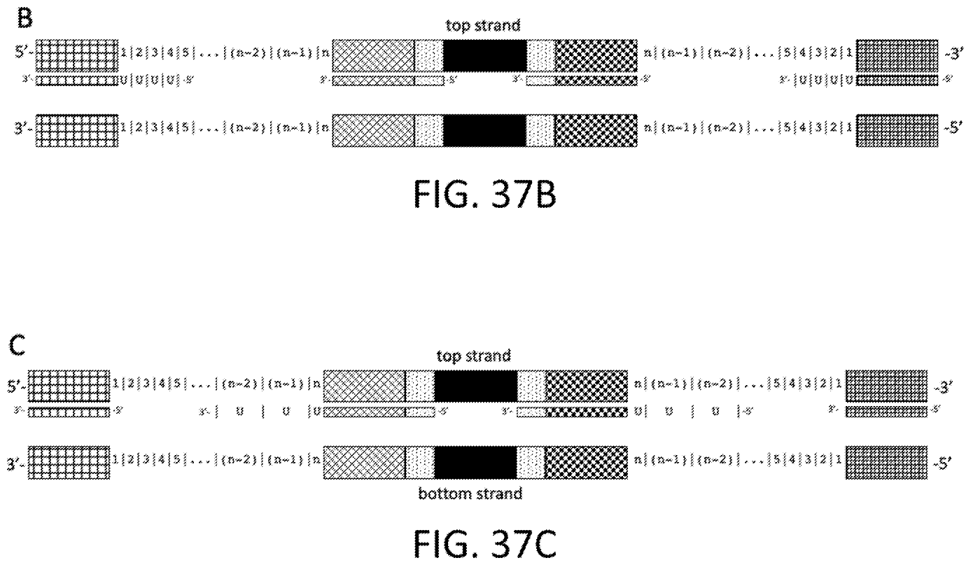

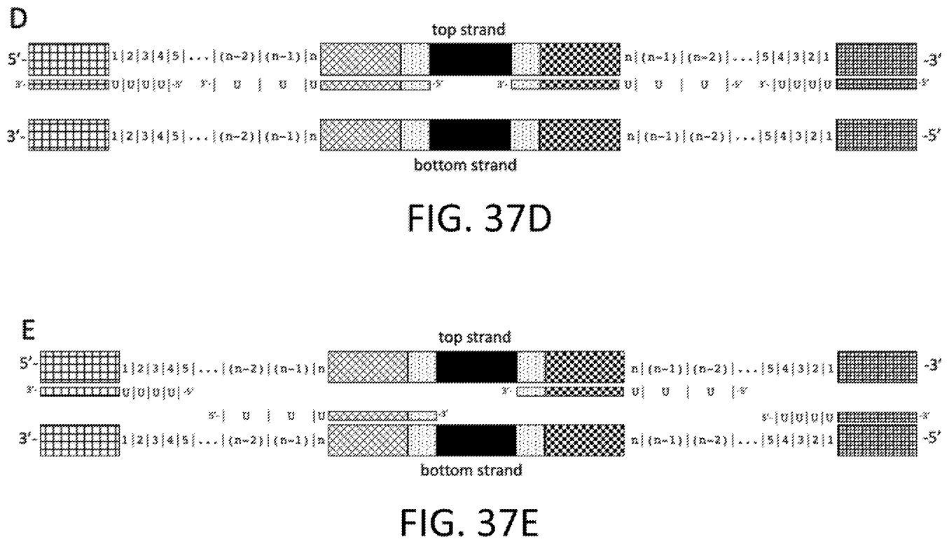

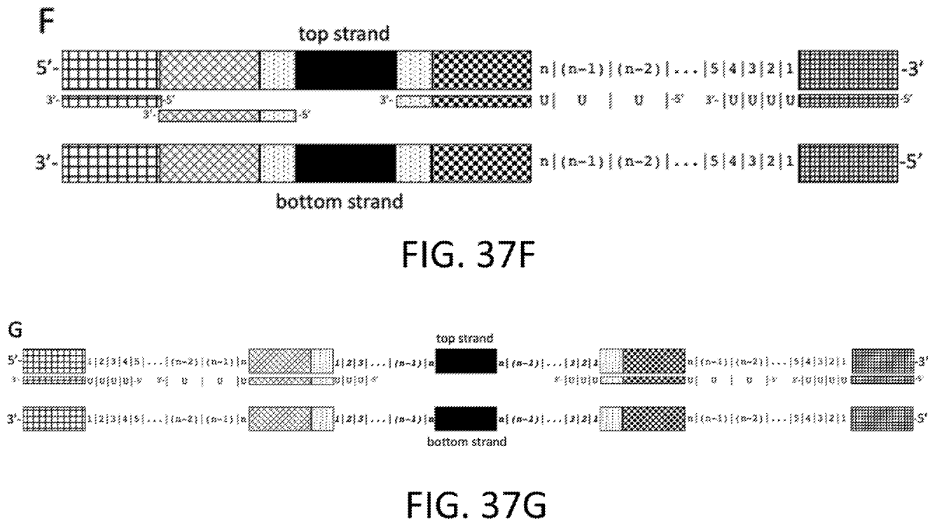

In a first arrangement, the length of the adapter index overhang may be varied. When designed from a single side, the adapter index overhang can be altered to cover from 0 to n of the adapter index bases from either side of the index (FIGS. 37B-37F). This allows for the ability to design such adapter blockers for both single (FIG. 37F) and dual index adapter systems (FIGS. 37B and 37C).

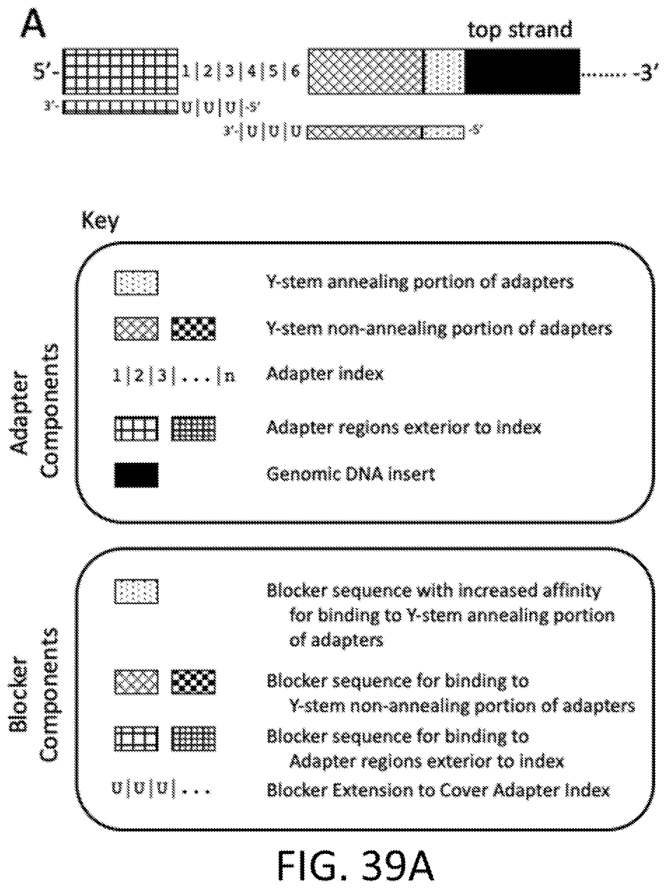

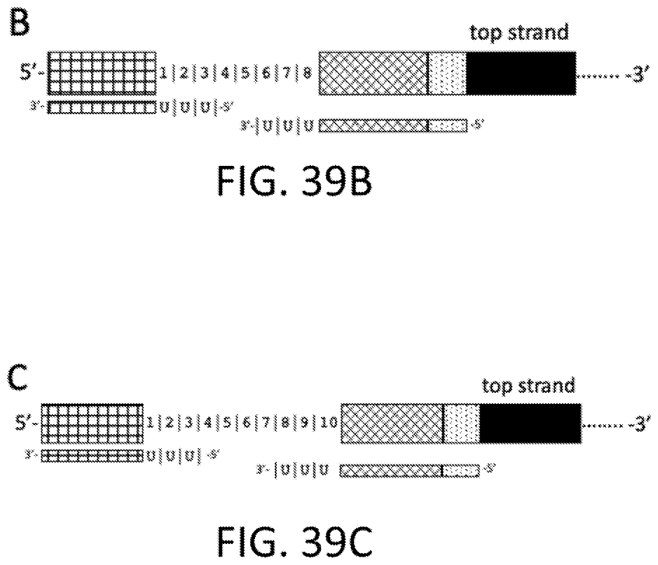

In a second arrangement, the adapter index bases are covered from both sides (FIGS. 37D and 37E). When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively (FIGS. 39A-39C).

In a third arrangement, modified nucleobases are selected to cover index adapter bases. Examples of these modifications that are currently commercially available include degenerate bases (i.e., mixed bases of A, T, C, G), 2′-deoxylnosine, & 5-nitroindole.

In a forth arrangement, blockers with adapter index overhangs bind to either the sense (i.e., ‘top’) or anti-sense (i.e., ‘bottom’) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition (FIG. 37G). These types of sequences can be placed in multiple locations of an adapter and in this case the most widely utilized case (i.e., unique molecular index next to the genomic insert) is presented. Other positions for the unique molecular identifier (e.g., next to adapter index bases) could also be addressed with similar approaches.

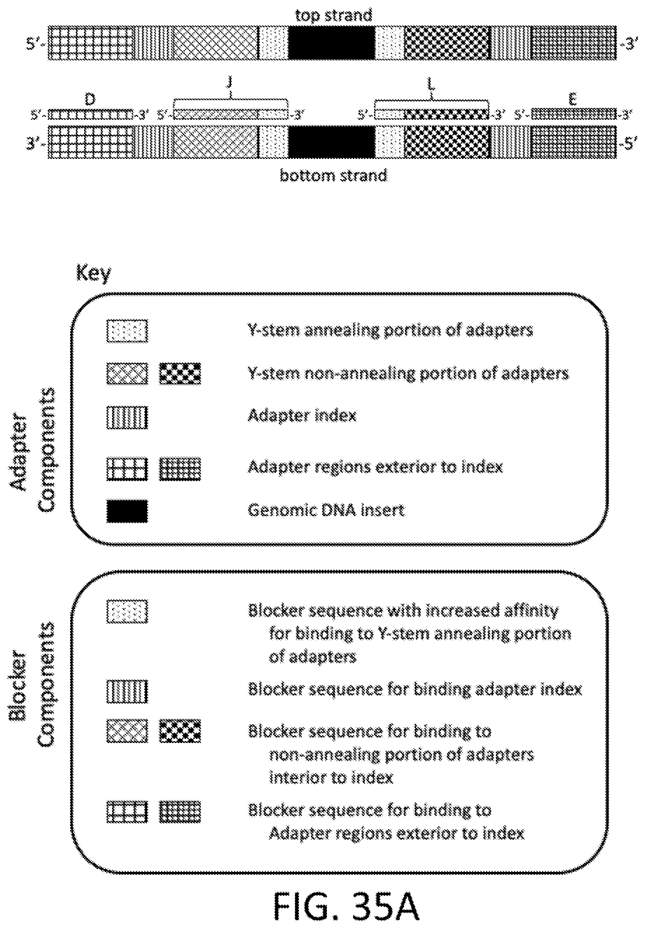

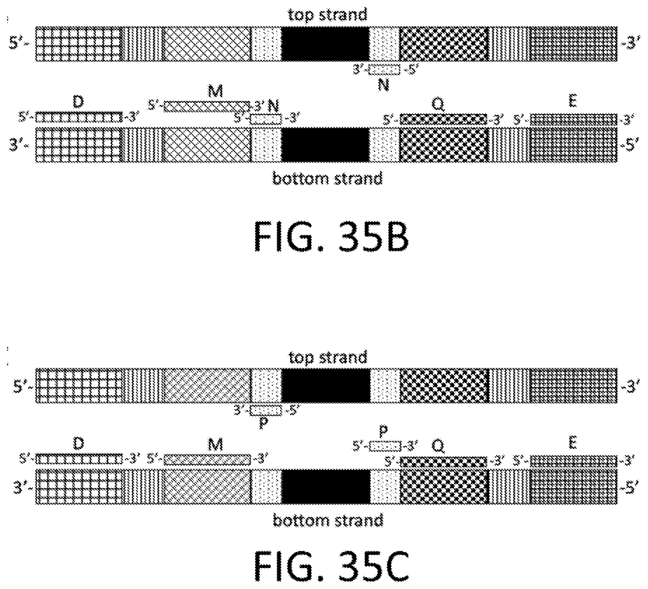

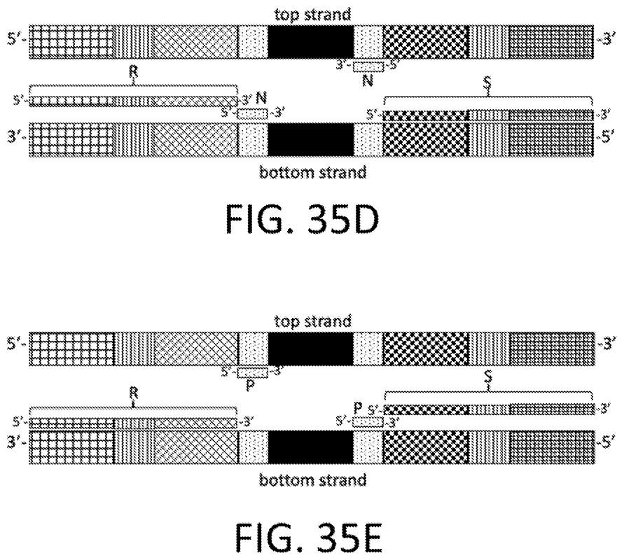

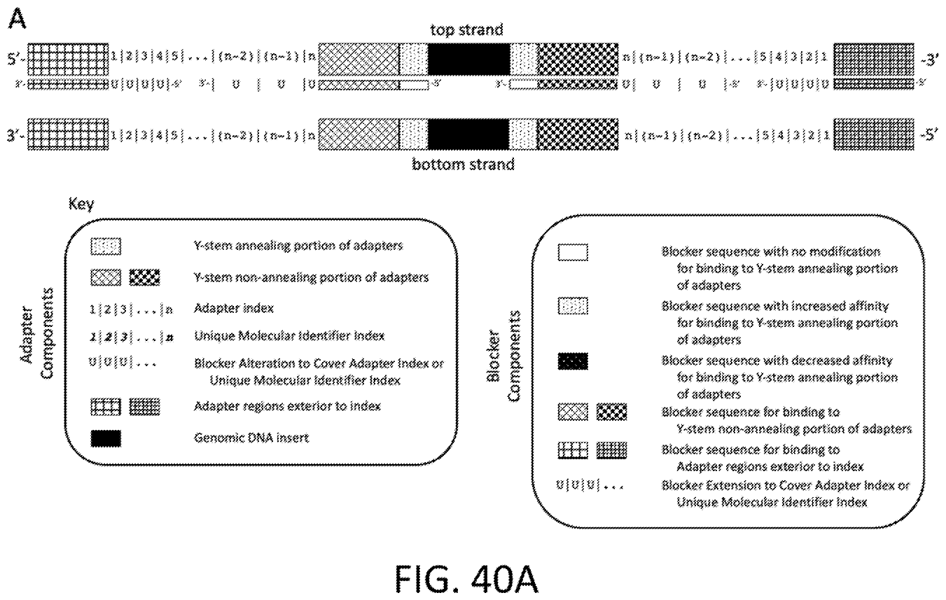

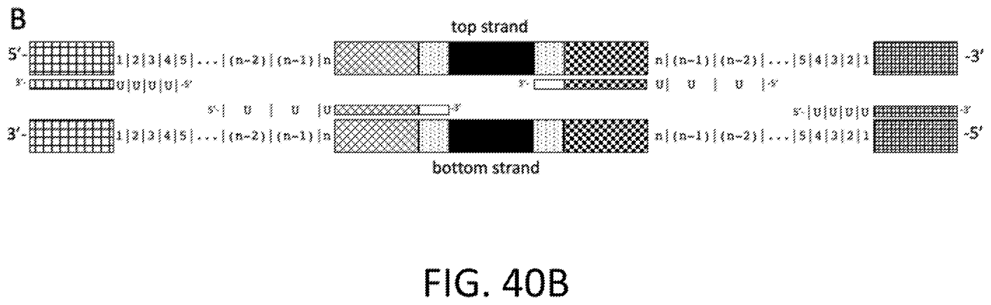

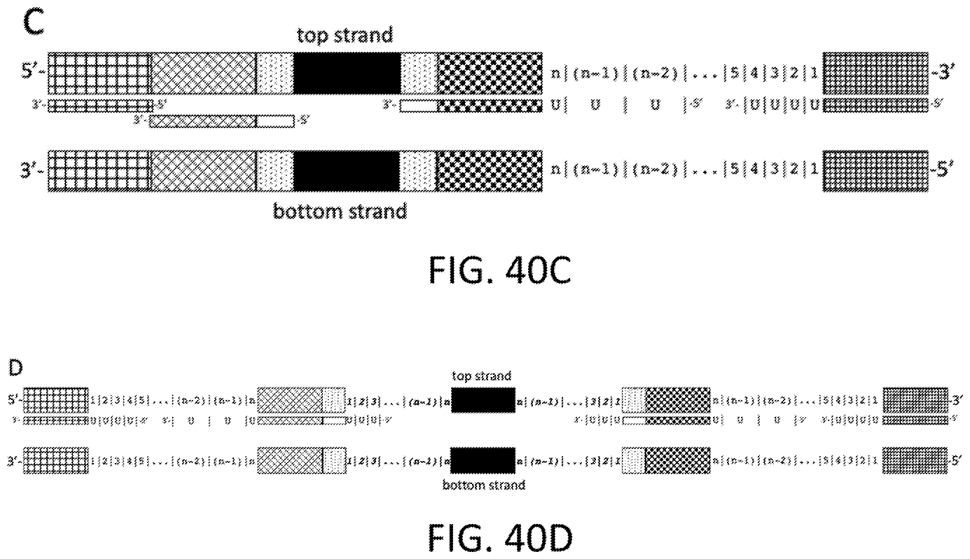

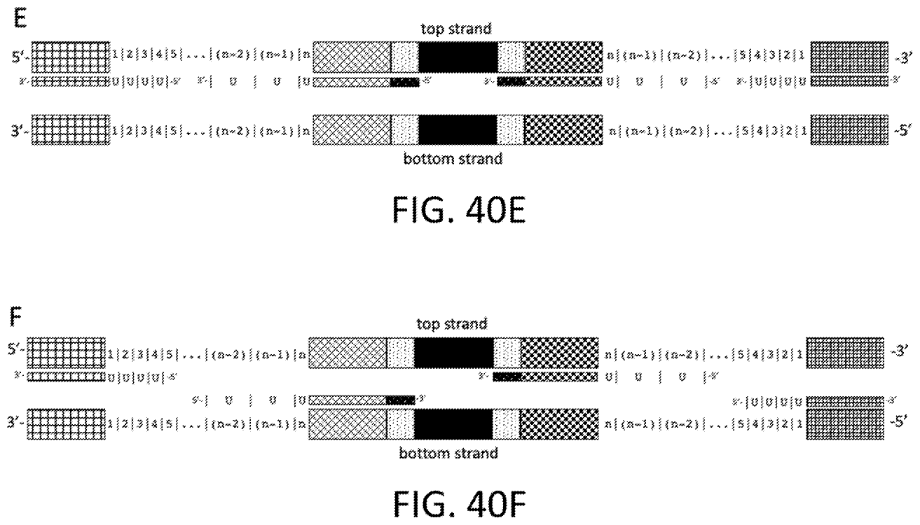

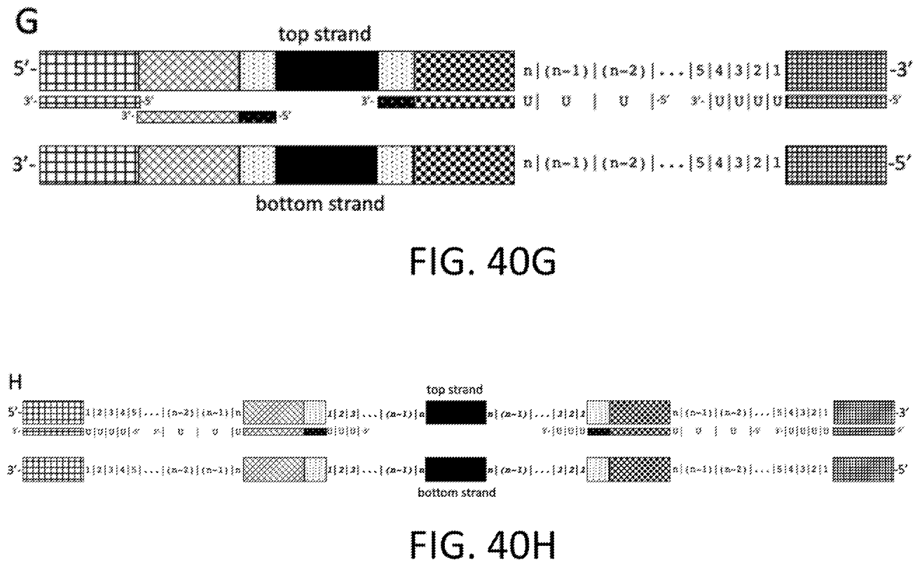

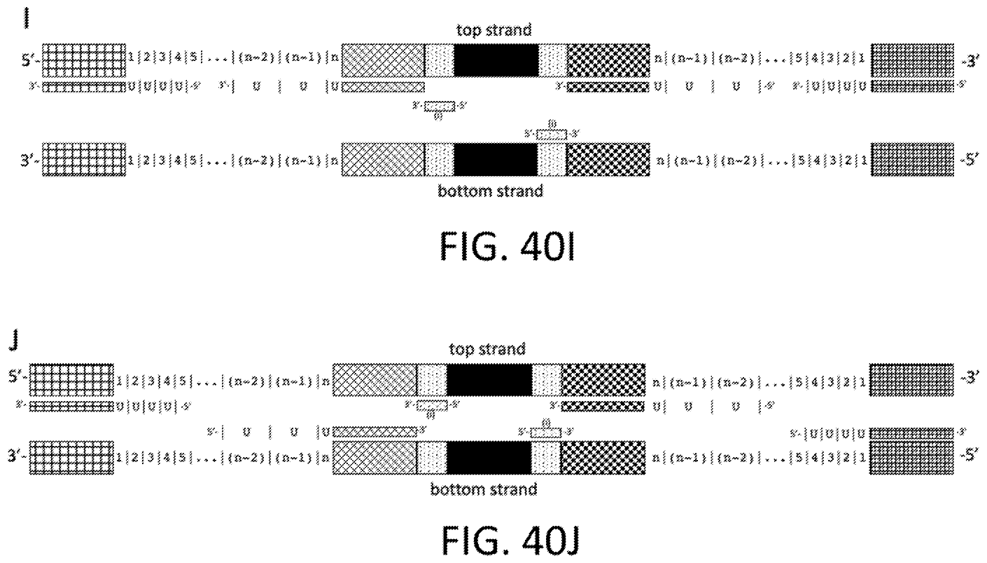

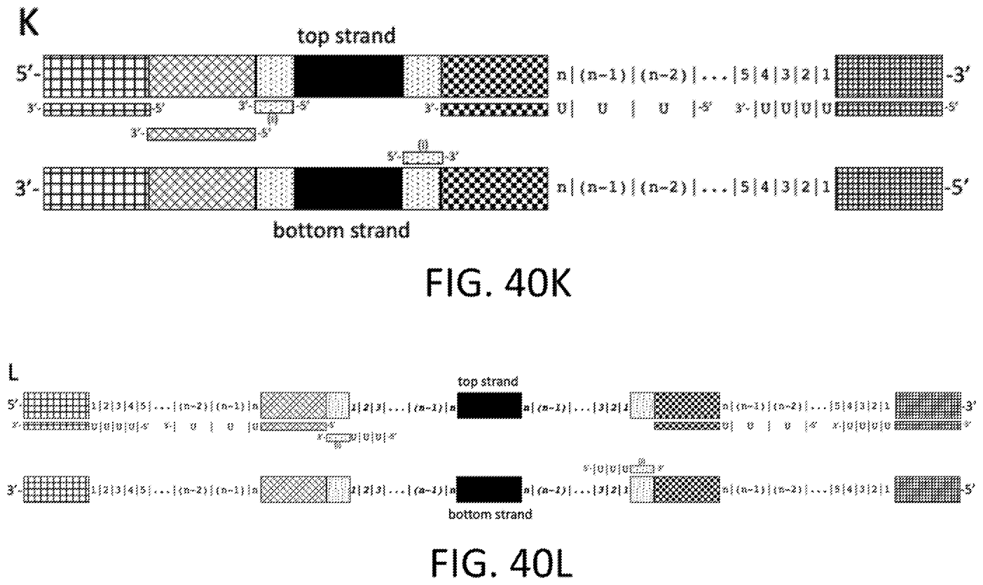

In a sixth arrangement, all of the previous arrangements are utilized in various combinations to meet a targeted performance metric for off-bait performance during target enrichment under specified conditions. In some instances, blockers comprise an arrangement shown in FIG. 35A. In some instances, blockers comprise an arrangement shown in FIG. 35B. In some instances, blockers comprise an arrangement shown in FIG. 35C. In some instances, blockers comprise an arrangement shown in FIG. 35D. In some instances, blockers comprise an arrangement shown in FIG. 35E. In some instances, blockers comprise an arrangement shown in FIG. 37A. In some instances, blockers comprise an arrangement shown in FIG. 37B. In some instances, blockers comprise an arrangement shown in FIG. 37C. In some instances, blockers comprise an arrangement shown in FIG. 37D. In some instances, blockers comprise an arrangement shown in FIG. 37E. In some instances, blockers comprise an arrangement shown in FIG. 37F. In some instances, blockers comprise an arrangement shown in FIG. 37G. In some instances, blockers comprise an arrangement shown in FIG. 39A. In some instances, blockers comprise an arrangement shown in FIG. 39B. In some instances, blockers comprise an arrangement shown in FIG. 39C. In some instances, blockers comprise an arrangement shown in FIG. 40A. In some instances, blockers comprise an arrangement shown in FIG. 40B. In some instances, blockers comprise an arrangement shown in FIG. 40C. In some instances, blockers comprise an arrangement shown in FIG. 40D. In some instances, blockers comprise an arrangement shown in FIG. 40E. In some instances, blockers comprise an arrangement shown in FIG. 40F. In some instances, blockers comprise an arrangement shown in FIG. 40G. In some instances, blockers comprise an arrangement shown in FIG. 40H. In some instances, blockers comprise an arrangement shown in FIG. 40I. In some instances, blockers comprise an arrangement shown in FIG. 40J. In some instances, blockers comprise an arrangement shown in FIG. 40K. In some instances, blockers comprise an arrangement shown in FIG. 40L.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxylnosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tm for binding to a cognate nucleobase. In some instances, universal bases comprise 5-nitroindole or 2′-deoxylnosine. In instances, blockers comprise spacer elements that connect two polynucleotide chains. In some instances, blockers comprise one or more nucleobase analogues selected from Table 1. In some instances, such nucleobase analogues are added to control the Tₘ of a blocker. Blockers may comprise any number of nucleobase analogues (such as LNAs or BNAs), depending on the desired hybridization Tₘ. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tₘ in a range of about 2° C. to about 8° C. for each nucleobase analogue. In some instances, the Tₘ is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tₘ. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tₘ to the adapter sequence. In some instances, non-canonical nucleic acids (for example locked nucleic acids, bridged nucleic acids, or other non-canonical nucleic acid or analog) are inserted into blockers to increase or decrease the blocker's Tₘ. In some instances, the Tₘ of a blocker is calculated using a tool specific to calculating Tₘ for polynucleotides comprising a non-canonical amino acid. In some instances, a Tₘ is calculated using the Exiqon™ online prediction tool. In some instances, blocker Tₘ described herein are calculated in-silico. In some instances, the blocker Tₘ is calculated in-silico, and is correlated to experimental in-vitro conditions. Without being bound by theory, an experimentally determined Tₘ may be further influenced by experimental parameters such as salt concentration, temperature, presence of additives, or other factor. In some instances, Tₘ described herein are in-silico determined Tₘ that are used to design or optimize blocker performance. In some instances, Tₘ values are predicted, estimated, or determined from melting curve analysis experiments. In some instances, blockers have a Tₘ of 70 degrees C. to 99 degrees C. In some instances, blockers have a Tₘ of 75 degrees C. to 90 degrees C. In some instances, blockers have a Tₘ of at least 85 degrees C. In some instances, blockers have a Tₘ of at least 70, 72, 75, 77, 80, 82, 85, 88, 90, or at least 92 degrees C. In some instances, blockers have a Tₘ of about 70, 72, 75, 77, 80, 82, 85, 88, 90, 92, or about 95 degrees C. In some instances, blockers have a Tₘ of 78 degrees C. to 90 degrees C. In some instances, blockers have a Tₘ of 79 degrees C. to 90 degrees C. In some instances, blockers have a Tₘ of 80 degrees C. to 90 degrees C. In some instances, blockers have a Tₘ of 81 degrees C. to 90 degrees C. In some instances, blockers have a Tₘ of 82 degrees C. to 90 degrees C. In some instances, blockers have a Tₘ of 83 degrees C. to 90 degrees C. In some instances, blockers have a Tₘ of 84 degrees C. to 90 degrees C. In some instances, a set of blockers have an average Tₘ of 78 degrees C. to 90 degrees C. In some instances, a set of blockers have an average Tₘ of 80 degrees C. to 90 degrees C. In some instances, a set of blockers have an average Tₘ of at least 80 degrees C. In some instances, a set of blockers have an average Tₘ of at least 81 degrees C. In some instances, a set of blockers have an average Tₘ of at least 82 degrees C. In some instances, a set of blockers have an average Tₘ of at least 83 degrees C. In some instances, a set of blockers have an average Tₘ of at least 84 degrees C. In some instances, a set of blockers have an average Tₘ of at least 86 degrees C. Blocker Tₘ are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker:target). In some instances, no more than 20% off-target reads are achieved with a molar ratio of less than 10:1 (blocker:target). In some instances, no more than 20% off-target reads are achieved with a molar ratio of less than 5:1 (blocker:target). In some instances, no more than 20% off-target reads are achieved with a molar ratio of less than 2:1 (blocker:target). In some instances, no more than 20% off-target reads are achieved with a molar ratio of less than 1.5:1 (blocker:target). In some instances, no more than 20% off-target reads are achieved with a molar ratio of less than 1.2:1 (blocker:target). In some instances, no more than 20% off-target reads are achieved with a molar ratio of less than 1.05:1 (blocker:target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5% 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

Methods for Sequencing

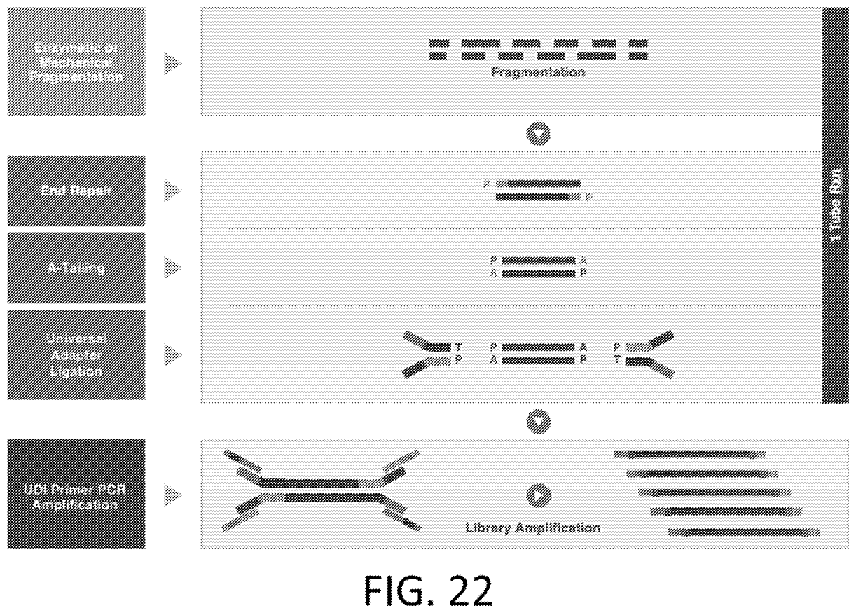

Described herein are methods to improve the efficiency and accuracy of sequencing. Such methods comprise use of universal adapters comprising nucleobase analogues, and generation of barcoded adapters after ligation to sample nucleic acids. In some instances, a sample is fragmented, fragment ends are repaired, one or more adenines is added to one strand of a fragment duplex, universal adapters are ligated, and a library of fragments is amplified with barcoded primers to generate a barcoded nucleic acid library (FIG. 22). Additional steps in some instances include enrichment/capture, additional PCR amplification, and/or sequencing of the nucleic acid library.

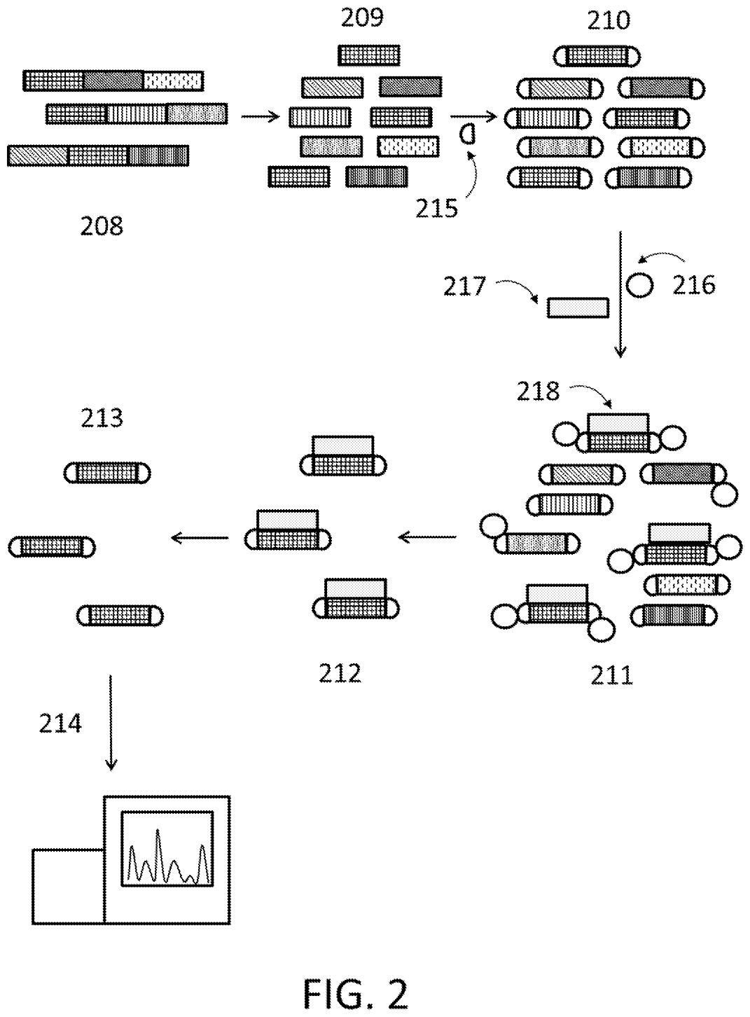

In a first step of an exemplary sequencing workflow (FIG. 2), a sample 208 comprising sample nucleic acids is fragmented by mechanical or enzymatic shearing to form a library of fragments 209. Indexed adapters 215 are ligated to fragmented sample nucleic acids to form an adapter-ligated sample nucleic acid library 210. This library is then optionally amplified. The library 210 is then optionally hybridized with target binding polynucleotides 217, which hybridize to sample nucleic acids 211, and hybridized with blocking polynucleotides 216 that prevent hybridization between sample nucleic acids 217 and adapters 215. Capture of sample nucleic acids-target binding polynucleotide hybridization pairs 212/218, and removal of target binding polynucleotides 217 allows isolation/enrichment of sample nucleic acids 213, which are then optionally amplified and sequenced 214.

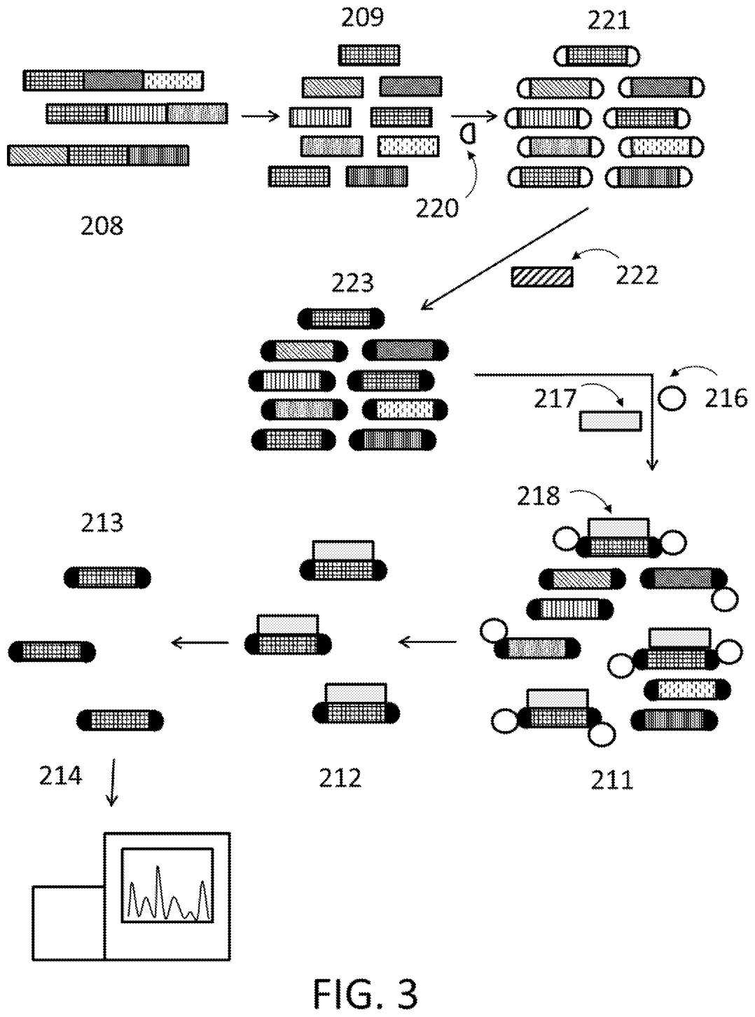

In a first step of an exemplary sequencing workflow (FIG. 3), a sample 208 comprising sample nucleic acids is fragmented by mechanical or enzymatic shearing to form a library of fragments 209. Universal adapters 220 are ligated to fragmented sample nucleic acids to form an adapter-ligated sample nucleic acid library 221. This library is then amplified with a barcoded primer library 222 (only one primer shown for simplicity) to generate a barcoded adapter-sample polynucleotide library 223. The library 223 is then optionally hybridized with target binding polynucleotides 217, which hybridize to sample nucleic acids, along with blocking polynucleotides 216 that prevent hybridization between probe polynucleotides 217 and adapters 220. Capture of sample polynucleotide-target binding polynucleotide hybridization pairs 212/218, and removal of target binding polynucleotides 217 allows isolation/enrichment of sample nucleic acids 213, which are then optionally amplified and sequenced 214. Various combinations of universal adapters and barcoded primers may be used. In some instances, barcoded primers comprise at least one barcode. In some instances, different types of barcodes are added to the sample nucleic acid using adapters or barcodes, or both. For example, a universal adapter comprises an index barcode, and after ligation is amplified with a barcoded primer comprising an additional index barcode. In some instances, a universal adapter comprises a unique molecular identifier barcode, and after ligation is amplified with a barcoded primer comprising an index barcode.

Barcoded primers may be used to amplify universal adapter-ligated sample polynucleotides using PCR, to generate a polynucleic acid library for sequencing. Such a library comprises barcodes after amplification in some instances. In some instances, amplification with barcoded primers results in higher amplification yields relative to amplification of a standard Y adapter-ligated sample polynucleotide library. In some instances, 2, 3, 4, 5, 6, 7, 8, 9, 10, 11, or 12 PCR cycles are used to amplify a universal adapter-ligated sample polynucleotide library. In some instances, no more than 2, 3, 4, 5, 6, 7, 8, 9, 10, 11, or no more than 12 PCR cycles are used to amplify a universal adapter-ligated sample polynucleotide library. In some instances, 2-12, 3-10, 4-9, 5-8, 6-10, or 8-12 PCR cycles are used to amplify a universal adapter-ligated sample polynucleotide library, thus generating amplicon products. Such libraries in some instances comprise fewer PCR-based errors. Without being bound by theory, reduced PCR cycles during amplification leads to fewer errors in resulting amplicon products. After amplification, such barcoded amplicon libraries are in some instances enriched or subjected to capture, additional amplification reactions, and/or sequencing. In some instances, amplicon products generated using the universal adapters described herein comprise about 30%, 15%, 10%, 7%, 5%, 3%, 2%, 1.5%, 1%, 0.5%, 0.1%, or 0.05% fewer errors than amplicon products generated from amplification of standard full-length Y adapters.

Described herein are methods wherein universal blockers are used to prevent off-target binding of capture probes to adapters ligated to genomic fragments, or adapter-adapter hybridization. Adapter blockers used for preventing off-target hybridization may target a portion or the entire adapter. In some instances, specific blockers are used that are complementary to a portion of the adapter that includes the unique index sequence. In cases where the adapter-tagged genomic library comprises a large number of different indices, it can be beneficial to design blockers which either do not target the index sequence, or do not hybridize strongly to it. For example, a “universal” blocker targets a portion of the adapter that does not comprise an index sequence (index independent), which allows a minimum number of blockers to be used regardless of the number of different index sequences employed. In some instances, no more than 8 universal blockers are used. In some instances, 4 universal blockers are used. In some instances, 3 universal blockers are used. In some instances, 2 universal blockers are used. In some instances, 1 universal blocker is used. In an exemplary arrangement, 4 universal blockers are used with adapters comprising at least 4, 8, 16, 32, 64, 96, or at least 128 different index sequences. In some instances, the different index sequences comprises at least or about 4, 6, 8, 10, 12, 14, 16, 18, 20, or more than 20 base pairs (bp). In some instances, a universal blocker is not configured to bind to a barcode sequence. In some instances, a universal blocker partially binds to a barcode sequence. In some instances, a universal blocker which partially binds to a barcode sequence further comprises nucleotide analogs, such as those that increase the Tₘ of binding to the adapter (e.g., LNAs or BNAs).

Methylation Sequencing and Capture

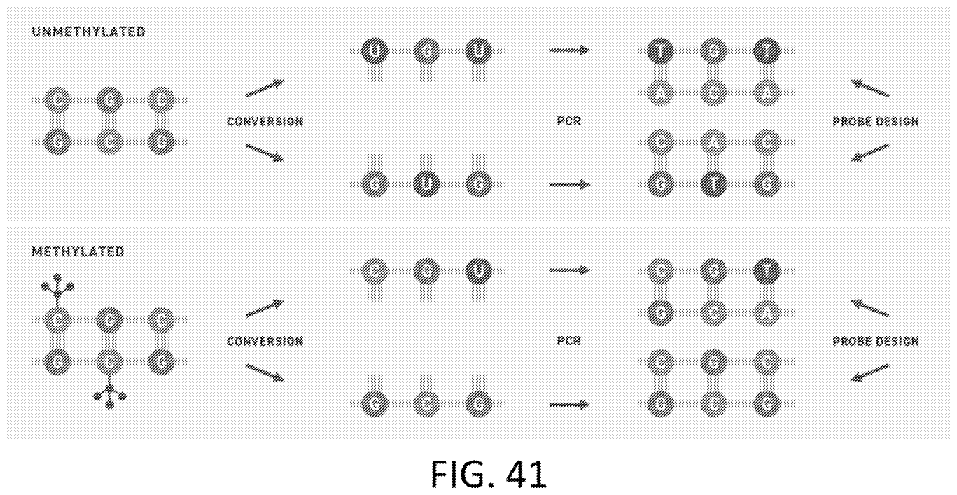

Methylation sequencing involves enzymatic or chemical methods leading to the conversion of unmethylated cytosines to uracil through a series of events culminating in deamination, while leaving methylated cytosines intact (FIG. 41). During amplification, uracils are paired with adenines on the complementary strand, leading to the inclusion of thymine in the original position of the unmethylated cytosine. In FIG. 41, there are identical sequences with each having unmethylated-cytosines in different positions. The end product is asymmetric, yielding two different double stranded DNA molecules after conversion (top row, FIG. 41); the same process for methylated DNA leads to yet additional sets of sequences (bottom row, FIG. 41).

Target enrichment can proceed by pre- or post-capture conversion. Post-capture conversion targets the original sample DNA to the left, while pre-capture targets the four strands of converted sequences on the right (FIG. 41). While post-capture conversion presents fewer challenges for probe design, it often requires large quantities of starting DNA material as PCR amplification does not preserve methylation patterns and cannot be performed before capture. Therefore, pre-capture conversion is often the method of choice for low-input, sensitive applications such as cell free DNA.

Methods described herein may comprise treatment of a library with enzymes or bisulfite to facilitate conversion of cytosines to uracil. In some instances, adapters (e.g., universal adapters) described herein comprise methylated nucleobases, such as methylated cytosine.

De Novo Synthesis of Small Polynucleotide Populations for Amplification Reactions

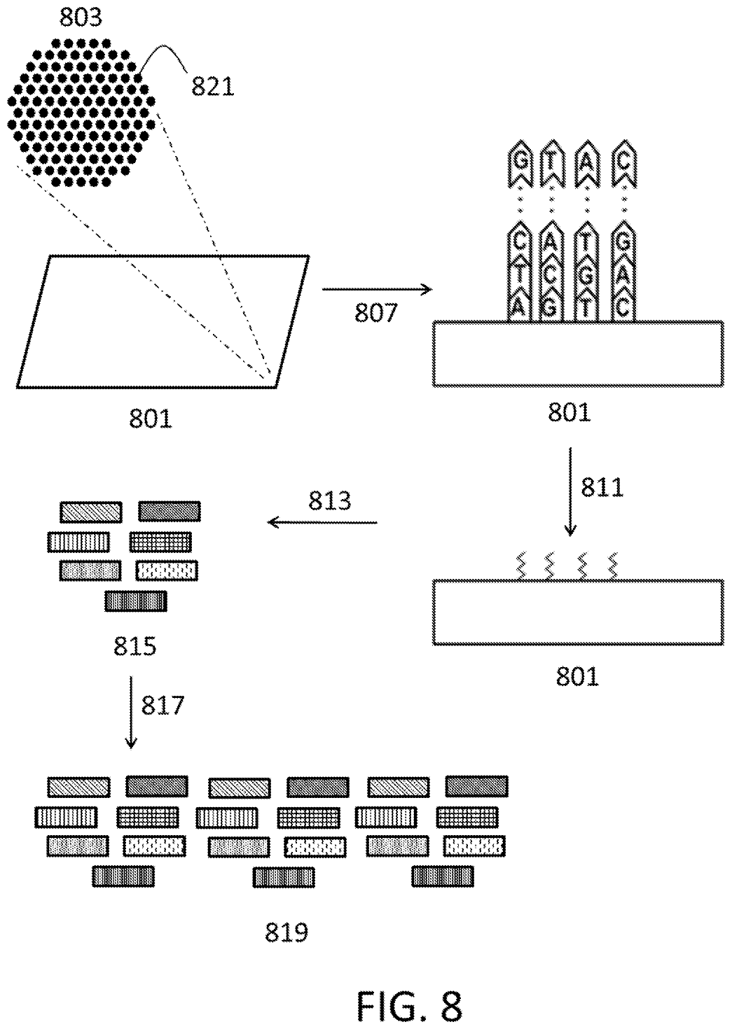

Described herein are methods of synthesis of polynucleotides from a surface, e.g., a plate. In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 8. A silicon plate 801 includes multiple clusters 803. Within each cluster are multiple loci 821. Polynucleotides are synthesized 807 de novo on a plate 801 from the cluster 803. Polynucleotides are cleaved 811 and removed 813 from the plate to form a population of released polynucleotides 815. The population of released polynucleotides 815 is then amplified 817 to form a library of amplified polynucleotides 819.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5× 1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

Polynucleotide Probe Structures

Libraries of polynucleotide probes can be used to enrich particular target sequences in a larger population of sample polynucleotides. In some instances, polynucleotide probes each comprise a target binding sequence complementary to one or more target sequences, one or more non-target binding sequences, and one or more primer binding sites, such as universal primer binding sites. Target binding sequences that are complementary or at least partially complementary in some instances bind (hybridize) to target sequences. Primer binding sites, such as universal primer binding sites facilitate simultaneous amplification of all members of the probe library, or a subpopulation of members. In some instances, the probes or adapters further comprise a barcode or index sequence. Barcodes are nucleic acid sequences that allow some feature of a polynucleotide with which the barcode is associated to be identified. After sequencing, the barcode region provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, 512, 1024, 2000, 5000, or more than 5000 barcoded libraries are used. In some instances, the polynucleotides are ligated to one or more molecular (or affinity) tags such as a small molecule, peptide, antigen, metal, or protein to form a probe for subsequent capture of the target sequences of interest. In some instances, only a portion of the polynucleotides are ligated to a molecular tag. In some instances, two probes that possess complementary target binding sequences which are capable of hybridization form a double stranded probe pair. Polynucleotide probes or adapters may comprise unique molecular identifiers (UMI). UMIs allow for internal measurement of initial sample concentrations or stoichiometry prior to downstream sample processing (e.g., PCR or enrichment steps) which can introduce bias. In some instances, UMIs comprise one or more barcode sequences.

Probes described here may be complementary to target sequences which are sequences in a genome. Probes described here may be complementary to target sequences which are exome sequences in a genome. Probes described here may be complementary to target sequences which are intron sequences in a genome. In some instances, probes comprise a target binding sequence complementary to a target sequence (of the sample nucleic acid), and at least one non-target binding sequence that is not complementary to the target. In some instances, the target binding sequence of the probe is about 120 nucleotides in length, or at least 10, 15, 20, 25, 50, 75, 100, 110, 120, 125, 140, 150, 160, 175, 200, 300, 400, 500, or more than 500 nucleotides in length. The target binding sequence is in some instances no more than 10, 15, 20, 25, 50, 75, 100, 125, 150, 175, 200, or no more than 500 nucleotides in length. The target binding sequence of the probe is in some instances about 120 nucleotides in length, or about 10, 15, 20, 25, 40, 50, 60, 70, 80, 85, 87, 90, 95, 97, 100, 105, 110, 115, 117, 118, 119, 120, 121, 122, 123, 124, 125, 126, 127, 128, 129, 130, 135, 140, 145, 150, 155, 157, 158, 159, 160, 161, 162, 163, 164, 165, 166, 167, 168, 169, 170, 175, 180, 190, 200, 210, 220, 230, 240, 250, 300, 400, or about 500 nucleotides in length. The target binding sequence is in some instances about 20 to about 400 nucleotides in length, or about 30 to about 175, about 40 to about 160, about 50 to about 150, about 75 to about 130, about 90 to about 120, or about 100 to about 140 nucleotides in length. The non-target binding sequence(s) of the probe is in some instances at least about 20 nucleotides in length, or at least about 1, 5, 10, 15, 17, 20, 23, 25, 50, 75, 100, 110, 120, 125, 140, 150, 160, 175, or more than about 175 nucleotides in length. The non-target binding sequence often is no more than about 5, 10, 15, 20, 25, 50, 75, 100, 125, 150, 175, or no more than about 200 nucleotides in length. The non-target binding sequence of the probe often is about 20 nucleotides in length, or about 1, 2, 3, 4, 5, 6, 7, 8, 9, 10, 11, 12, 13, 14, 15, 16, 17, 18, 19, 20, 21, 22, 23, 25, 40, 50, 60, 70, 80, 90, 100, 110, 120, 130, 140, 150, or about 200 nucleotides in length. The non-target binding sequence in some instances is about 1 to about 250 nucleotides in length, or about 20 to about 200, about 10 to about 100, about 10 to about 50, about 30 to about 100, about 5 to about 40, or about 15 to about 35 nucleotides in length. The non-target binding sequence often comprises sequences that are not complementary to the target sequence, and/or comprise sequences that are not used to bind primers. In some instances, the non-target binding sequence comprises a repeat of a single nucleotide, for example polyadenine or polythymidine. A probe often comprises none or at least one non-target binding sequence. In some instances, a probe comprises one or two non-target binding sequences. The non-target binding sequence may be adjacent to one or more target binding sequences in a probe. For example, a non-target binding sequence is located on the 5′ or 3′ end of the probe. In some instances, the non-target binding sequence is attached to a molecular tag or spacer.

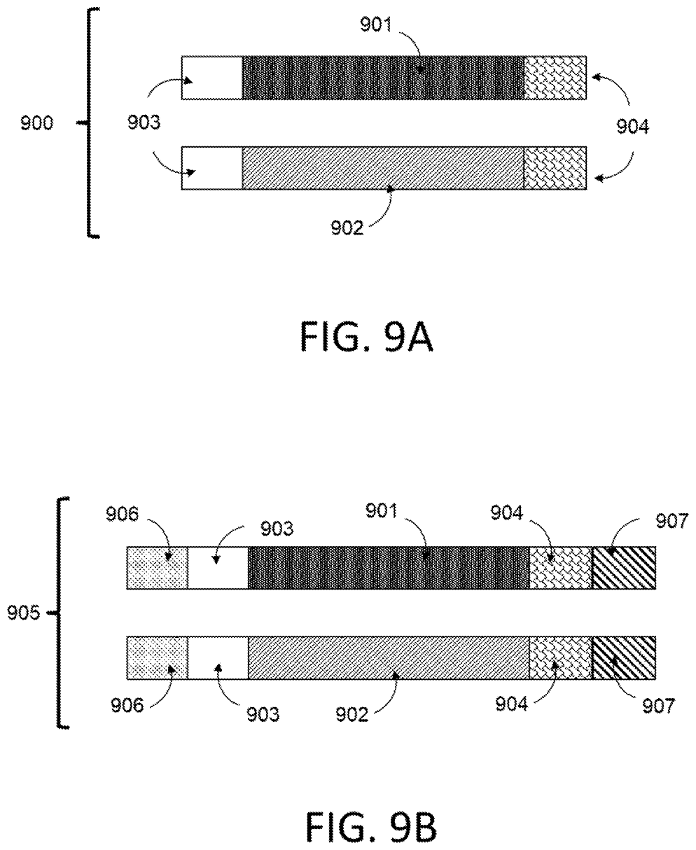

In some instances, the non-target binding sequence(s) may be a primer binding site. The primer binding sites often are each at least about 20 nucleotides in length, or at least about 10, 12, 14, 16, 18, 20, 22, 24, 26, 28, 30, 32, 34, 36, 38, or at least about 40 nucleotides in length. Each primer binding site in some instances is no more than about 10, 12, 14, 16, 18, 20, 22, 24, 26, 28, 30, 32, 34, 36, 38, or no more than about 40 nucleotides in length. Each primer binding site in some instances is about 10 to about 50 nucleotides in length, or about 15 to about 40, about 20 to about 30, about 10 to about 40, about 10 to about 30, about 30 to about 50, or about 20 to about 60 nucleotides in length. In some instances the polynucleotide probes comprise at least two primer binding sites. In some instances, primer binding sites may be universal primer binding sites, wherein all probes comprise identical primer binding sequences at these sites. In some instances, a pair of polynucleotide probes targeting a particular sequence and its reverse complement (e.g., a region of genomic DNA) are represented by 900 in FIG. 9A, comprising a first target binding sequence 901, a second target binding sequence 902, a first non-target binding sequence 903, and a second non-target binding sequence 904. For example, a pair of polynucleotide probes complementary to a particular sequence (e.g., a region of genomic DNA).

In some instances, the first target binding sequence 901 is the reverse complement of the second target binding sequence 902. In some instances, both target binding sequences are chemically synthesized prior to amplification. In an alternative arrangement, a pair of polynucleotide probes targeting a particular sequence and its reverse complement (e.g., a region of genomic DNA) are represented by 905 in FIG. 9B, comprising a first target binding sequence 901, a second target binding sequence 902, a first non-target binding sequence 903, a second non-target binding sequence 904, a third non-target binding sequence 906, and a fourth non-target binding sequence 907. In some instances, the first target binding sequence 901 is the reverse complement of the second target binding sequence 902. In some instances, one or more non-target binding sequences comprise polyadenine or polythymidine.

In some instances, both probes in the pair are labeled with at least one molecular tag. In some instances, PCR is used to introduce molecular tags (via primers comprising the molecular tag) onto the probes during amplification. In some instances, the molecular tag comprises one or more biotin, folate, a polyhistidine, a FLAG tag, glutathione, or other molecular tag consistent with the specification. In some instances probes are labeled at the 5′ terminus. In some instances, the probes are labeled at the 3′ terminus. In some instances, both the 5′ and 3′ termini are labeled with a molecular tag. In some instances, the 5′ terminus of a first probe in a pair is labeled with at least one molecular tag, and the 3′ terminus of a second probe in the pair is labeled with at least one molecular tag. In some instances, a spacer is present between one or more molecular tags and the nucleic acids of the probe. In some instances, the spacer may comprise an alkyl, polyol, or polyamino chain, a peptide, or a polynucleotide. The solid support used to capture probe-target nucleic acid complexes in some instances, is a bead or a surface. The solid support in some instances comprises glass, plastic, or other material capable of comprising a capture moiety that will bind the molecular tag. In some instances, a bead is a magnetic bead. For example, probes labeled with biotin are captured with a magnetic bead comprising streptavidin. The probes are contacted with a library of nucleic acids to allow binding of the probes to target sequences. In some instances, blocking polynucleic acids are added to prevent binding of the probes to one or more adapter sequences attached to the target nucleic acids. In some instances, blocking polynucleic acids comprise one or more nucleic acid analogues. In some instances, blocking polynucleic acids have a uracil substituted for thymine at one or more positions.

Probes described herein may comprise complementary target binding sequences which bind to one or more target nucleic acid sequences. In some instances, the target sequences are any DNA or RNA nucleic acid sequence. In some instances, target sequences may be longer than the probe insert. In some instance, target sequences may be shorter than the probe insert. In some instance, target sequences may be the same length as the probe insert. For example, the length of the target sequence may be at least or about at least 2, 10, 15, 20, 25, 30, 35, 40, 45, 50, 100, 150, 200, 300, 400, 500, 1000, 2000, 5,000, 12,000, 20,000 nucleotides, or more. The length of the target sequence may be at most or about at most 20,000, 12,000, 5,000, 2,000, 1,000, 500, 400, 300, 200, 150, 100, 50, 45, 35, 30, 25, 20, 19, 18, 17, 16, 15, 14, 13, 12, 11, 10, 2 nucleotides, or less. The length of the target sequence may fall from 2-20,000, 3-12,000, 5-5, 5000, 10-2,000, 10-1,000, 10-500, 9-400, 11-300, 12-200, 13-150, 14-100, 15-50, 16-45, 17-40, 18-35, and 19-25. The probe sequences may target sequences associated with specific genes, diseases, regulatory pathways, or other biological functions consistent with the specification.

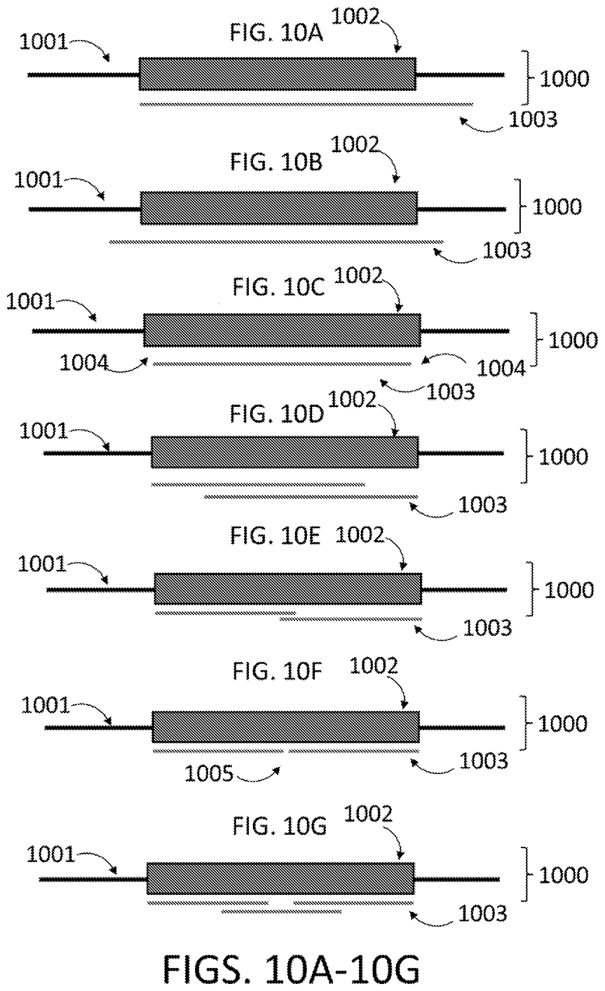

In some instances, a single probe insert 1003 is complementary to one or more target sequences 1002 (FIGS. 10A-10G) in a larger polynucleic acid 1000. An exemplary target sequence is an exon. In some instances, one or more probes target a single target sequence (FIGS. 10A-10G). In some instances, a single probe may target more than one target sequence. In some instances, the target binding sequence of the probe targets both a target sequence 1002 and an adjacent sequence 1001 (FIGS. 10A and 10B). In some instances, a first probe targets a first region and a second region of a target sequence, and a second probe targets the second region and a third region of the target sequence (FIG. 10D and FIG. 10E). In some instances, a plurality of probes targets a single target sequence, wherein the target binding sequences of the plurality of probes contain one or more sequences which overlap with regard to complementarity to a region of the target sequence (FIG. 10G). In some instances, probe inserts do not overlap with regard to complementarity to a region of the target sequence. In some instances, at least at least 2, 10, 15, 20, 25, 30, 35, 40, 45, 50, 100, 150, 200, 300, 400, 500, 1000, 2000, 5,000, 12,000, 20,000, or more than 20,000 probes target a single target sequence. In some instances no more than 4 probes directed to a single target sequence overlap, or no more than 3, 2, 1, or no probes targeting a single target sequence overlap. In some instances, one or more probes do not target all bases in a target sequence, leaving one or more gaps (FIG. 10C and FIG. 10F). In some instances, the gaps are near the middle of the target sequence 1005 (FIG. 10F). In some instances, the gaps 1004 are at the 5′ or 3′ ends of the target sequence (FIG. 10C). In some instances, the gaps are 6 nucleotides in length. In some instances, the gaps are no more than 1, 2, 3, 4, 5, 6, 7, 8, 9, 10, 20, 30, 40, or no more than 50 nucleotides in length. In some instances, the gaps are at least 1, 2, 3, 4, 5, 6, 7, 8, 9, 10, 20, 30, 40, or at least 50 nucleotides in length. In some instances, the gap length falls within 1-50, 1-40, 1-30, 1-20, 1-10, 2-30, 2-20, 2-10, 3-50, 3-25, 3-10, or 3-8 nucleotides in length. In some instances, a set of probes targeting a sequence do not comprise overlapping regions amongst probes in the set when hybridized to complementary sequence. In some instances, a set of probes targeting a sequence do not have any gaps amongst probes in the set when hybridized to complementary sequence. Probes may be designed to maximize uniform binding to target sequences. In some instances, probes are designed to minimize target binding sequences of high or low GC content, secondary structure, repetitive/palindromic sequences, or other sequence feature that may interfere with probe binding to a target. In some instances, a single probe may target a plurality of target sequences.

A probe library described herein may comprise at least 10, 20, 50, 100, 200, 500, 1,000, 2,000, 5,000, 10,000, 20,000, 50,000, 100,000, 200,000, 500,000, 1,000,000 or more than 1,000,000 probes. A probe library may have no more than 10, 20, 50, 100, 200, 500, 1,000, 2,000, 5,000, 10,000, 20,000, 50,000, 100,000, 200,000, 500,000, or no more than 1,000,000 probes. A probe library may comprise 10 to 500, 20 to 1000, 50 to 2000, 100 to 5000, 500 to 10,000, 1,000 to 5,000, 10,000 to 50,000, 100,000 to 500,000, or to 50,000 to 1,000,000 probes. A probe library may comprise about 370,000; 400,000; 500,000 or more different probes.

Next Generation Sequencing Applications

Downstream applications of polynucleotide libraries may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

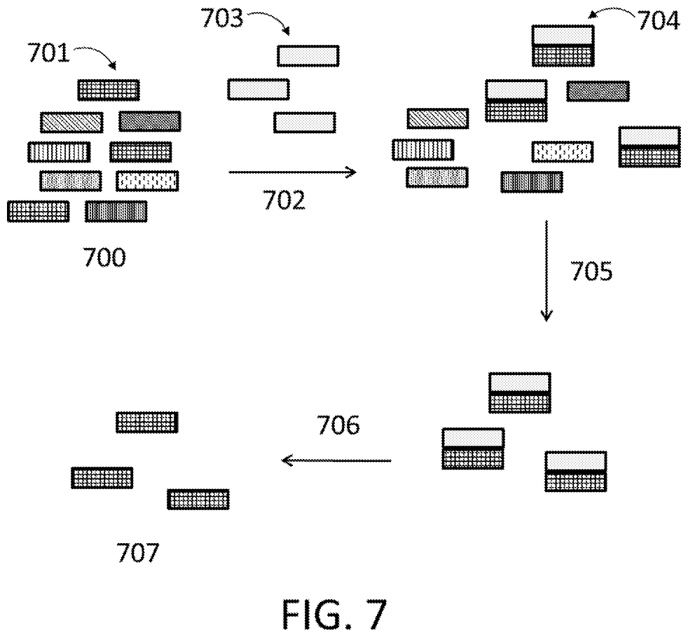

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. In some instances, an enrichment workflow is depicted in FIG. 7. A library 700 of double stranded adapter-tagged polynucleotide strands 701 is contacted with polynucleotide probes 702, to form hybrid pairs 704. Such pairs are separated 705 from unhybridized fragments, and isolated 706 from probes to produce an enriched library 707.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (i.e., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification. SMSS is described in part in US Publication Application Nos. 2006002471I; 20060024678; 20060012793; 20060012784; and 20050100932.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959; and well as in US Publication Application Nos. 20020012930; 20030058629; 20030100102; 20030148344; 20040248161; 20050079510, 20050124022; and 20060078909.

In some instances, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106130; 20030064398; 20030022207; and Constans, A., The Scientist 2003, 17(13):36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing {See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiN, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

Highly Parallel De Novo Nucleic Acid Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 um to about 500 um, from about 10 um to about 200 um, or from about 10 um to about 100 um. In some instances, the distance from two centers of adjacent loci is greater than about 10 um, 20 um, 30 um, 40 um, 50 um, 60 um, 70 um, 80 um, 90 um or 100 um. In some instances, the distance from the centers of two adjacent loci is less than about 200 um, 150 um, 100 um, 80 um, 70 um, 60 um, 50 um, 40 um, 30 um, 20 um or 10 um. In some instances, each locus has a width of about 0.5 um, 1 um, 2 um, 3 um, 4 um, 5 um, 6 um, 7 um, 8 um, 9 um, 10 um, 20 um, 30 um, 40 um, 50 um, 60 um, 70 um, 80 um, 90 um or 100 um. In some instances, each locus is has a width of about 0.5 um to 100 um, about 0.5 um to 50 um, about 10 um to 75 um, or about 0.5 um to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 um, 100 um, 200 um, 500 um, 1000 um, or 2000 um or 5000 um. In some instances, the distance from the centers of two adjacent clusters is from about 50 um and about 100 um, from about 50 um and about 200 um, from about 50 um and about 300 um, from about 50 um and about 500 um, and from about 100 um to about 2000 um. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 2%, 30%, 40%, 5%, 10%, 150%, 20%, 30%, 40%, 500%, 600%, 700%, 800%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 400, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um.

Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H-perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the 06 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150, 22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of polynucleotides comprising variant polynucleotides differing at a plurality of codon sites. In some instances, a polynucleotide may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may be not be adjacent and separated by 1, 2, 3, 4, 5, 6, 7, 8, 9, 10, or more codons.

In some instances, a polynucleotide may comprise multiple sites of variant codon sites, wherein all the variant codon sites are adjacent to one another, forming a stretch of variant codon sites. In some instances, a polynucleotide may comprise multiple sites of variant codon sites, wherein none the variant codon sites are adjacent to one another. In some instances, a polynucleotide may comprise multiple sites of variant codon sites, wherein some the variant codon sites are adjacent to one another, forming a stretch of variant codon sites, and some of the variant codon sites are not adjacent to one another.

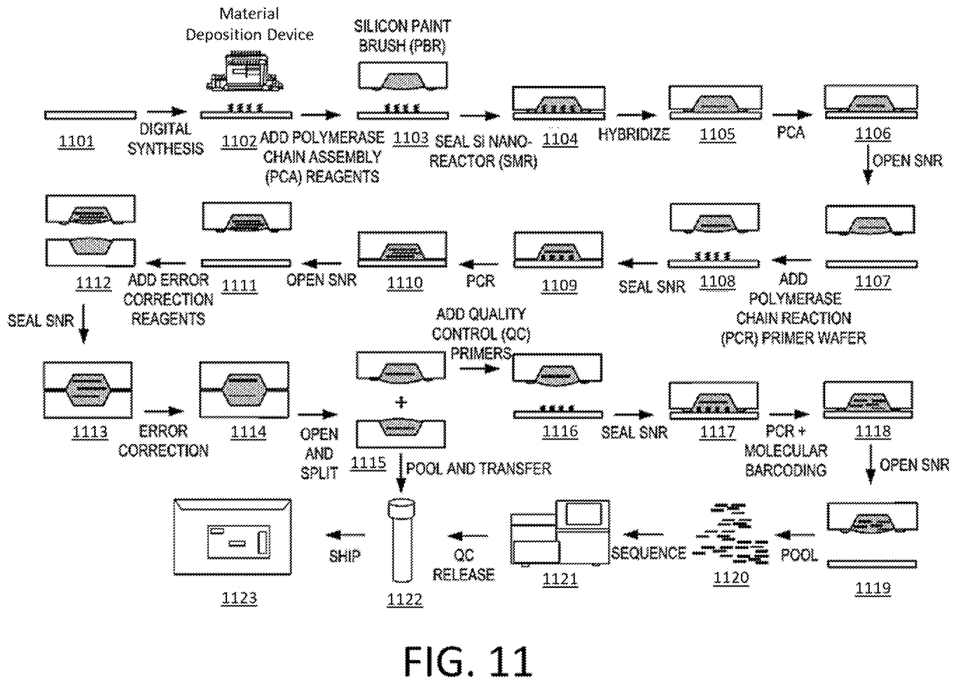

Referring to the Figures, FIG. 11 illustrates an exemplary process workflow for synthesis of nucleic acids (e.g., genes) from shorter polynucleotides. The workflow is divided generally into phases: (1) de novo synthesis of a single stranded polynucleotide library, (2) joining polynucleotides to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large polynucleotides for generation are selected, a predetermined library of polynucleotides is designed for de novo synthesis. Various suitable methods are known for generating high density polynucleotide arrays. In the workflow example, a device surface layer 1101 is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A material deposition device, such as a polynucleotide synthesizer, is designed to release reagents in a step wise fashion such that multiple polynucleotides extend, in parallel, one residue at a time to generate oligomers with a predetermined nucleic acid sequence 1102. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 1103. Prior to or after the sealing 1104 of the polynucleotides, a reagent is added to release the polynucleotides from the substrate. In the exemplary workflow, the polynucleotides are released subsequent to sealing of the nanoreactor 1105. Once released, fragments of single stranded polynucleotides hybridize in order to span an entire long range sequence of DNA. Partial hybridization 1105 is possible because each synthesized polynucleotide is designed to have a small portion overlapping with at least one other polynucleotide in the population.

After hybridization, a PCR reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 1106.

After PCR is complete, the nanoreactor is separated from the device 1107 and positioned for interaction with a device having primers for PCR 1108. After sealing, the nanoreactor is subject to PCR 1109 and the larger nucleic acids are amplified. After PCR 1110, the nanochamber is opened 1111, error correction reagents are added 1112, the chamber is sealed 1113 and an error correction reaction occurs to remove mismatched base pairs and/or strands with poor complementarity from the double stranded PCR amplification products 1114. The nanoreactor is opened and separated 1115. Error corrected product is next subject to additional processing steps, such as PCR and molecular bar coding, and then packaged 1122 for shipment 1123.

In some instances, quality control measures are taken. After error correction, quality control steps include for example interaction with a wafer having sequencing primers for amplification of the error corrected product 1116, sealing the wafer to a chamber containing error corrected amplification product 1117, and performing an additional round of amplification 1118. The nanoreactor is opened 1119 and the products are pooled 1120 and sequenced 1121. After an acceptable quality control determination is made, the packaged product 1122 is approved for shipment 1123.

In some instances, a nucleic acid generate by a workflow such as that in FIG. 11 is subject to mutagenesis using overlapping primers disclosed herein. In some instances, a library of primers are generated by in situ preparation on a solid support and utilize single nucleotide extension process to extend multiple oligomers in parallel. A deposition device, such as a polynucleotide synthesizer, is designed to release reagents in a step wise fashion such that multiple polynucleotides extend, in parallel, one residue at a time to generate oligomers with a predetermined nucleic acid sequence 1102.

Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

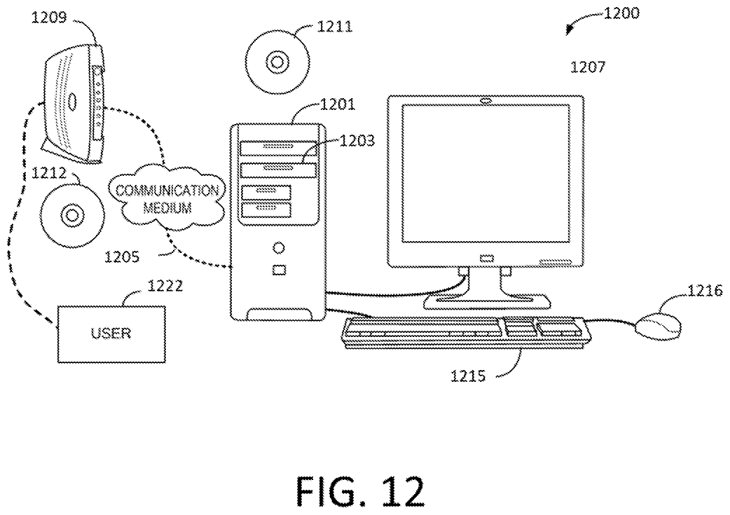

The computer system 1200 illustrated in FIG. 12 may be understood as a logical apparatus that can read instructions from media 1211 and/or a network port 1205, which can optionally be connected to server 1209 having fixed media 1212. The system, such as shown in FIG. 12 can include a CPU 1201, disk drives 1203, optional input devices such as keyboard 1215 and/or mouse 1216 and optional monitor 1207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1222 as illustrated in FIG. 12.

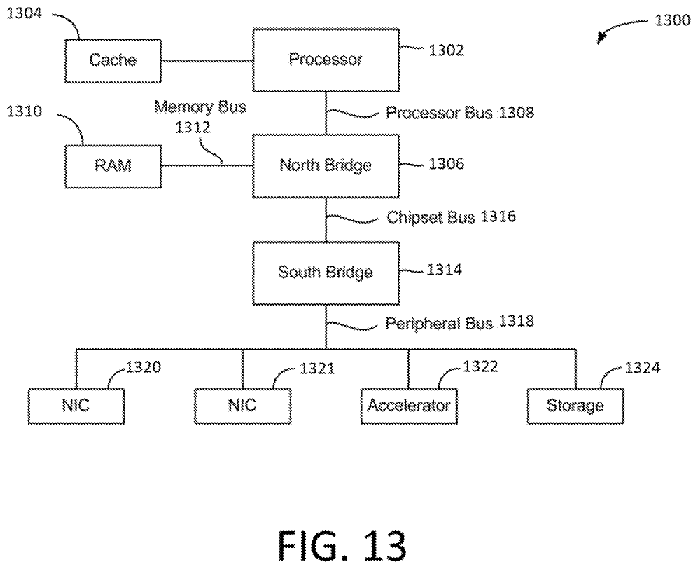

FIG. 13 is a block diagram illustrating a first example architecture of a computer system 1300 that can be used in connection with example instances of the present disclosure. As depicted in FIG. 13, the example computer system can include a processor 1302 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 13, a high speed cache 1304 can be connected to, or incorporated in, the processor 1302 to provide a high speed memory for instructions or data that have been recently, or are frequently, used by processor 1302. The processor 1302 is connected to a north bridge 1306 by a processor bus 1308. The north bridge 1306 is connected to random access memory (RAM) 1310 by a memory bus 1312 and manages access to the RAM 1310 by the processor 1302. The north bridge 1306 is also connected to a south bridge 1314 by a chipset bus 1316. The south bridge 1314 is, in turn, connected to a peripheral bus 1318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1318. In some alternative architectures, the functionality of the north bridge can be incorporated into the processor instead of using a separate north bridge chip. In some instances, system 1300 can include an accelerator card 1322 attached to the peripheral bus 1318. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1324 and can be loaded into RAM 1310 and/or cache 1304 for use by the processor. The system 1300 includes an operating system for managing system resources; non-limiting examples of operating systems include: Linux, Windows™, MACOS™, BlackBerry OS™ iOS™, and other functionally-equivalent operating systems, as well as application software running on top of the operating system for managing data storage and optimization in accordance with example instances of the present disclosure. In this example, system 1300 also includes network interface cards (NICs) 1320 and 1321 connected to the peripheral bus for providing network interfaces to external storage, such as Network Attached Storage (NAS) and other computer systems that can be used for distributed parallel processing.

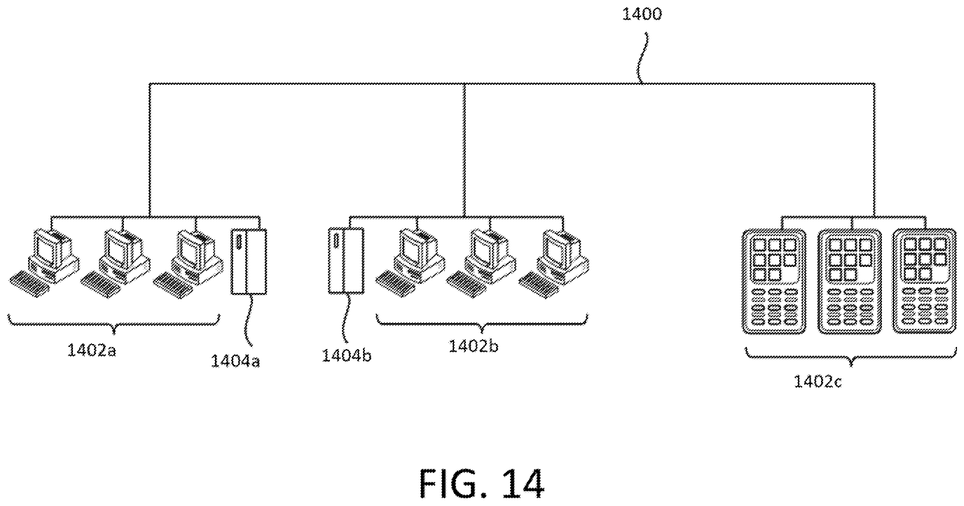

FIG. 14 is a diagram showing a network 1400 with a plurality of computer systems 1402a, and 1402b, a plurality of cell phones and personal data assistants 1402c, and Network Attached Storage (NAS) 1404a, and 1404b. In example instances, systems 1402a, 1402b, and 1402c can manage data storage and optimize data access for data stored in Network Attached Storage (NAS) 1404a and 1404b. A mathematical model can be used for the data and be evaluated using distributed parallel processing across computer systems 1402a, and 1402b, and cell phone and personal data assistant systems 1402c. Computer systems 1402a, and 1402b, and cell phone and personal data assistant systems 1402c can also provide parallel processing for adaptive data restructuring of the data stored in Network Attached Storage (NAS) 1404a and 1404b. FIG. 14 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

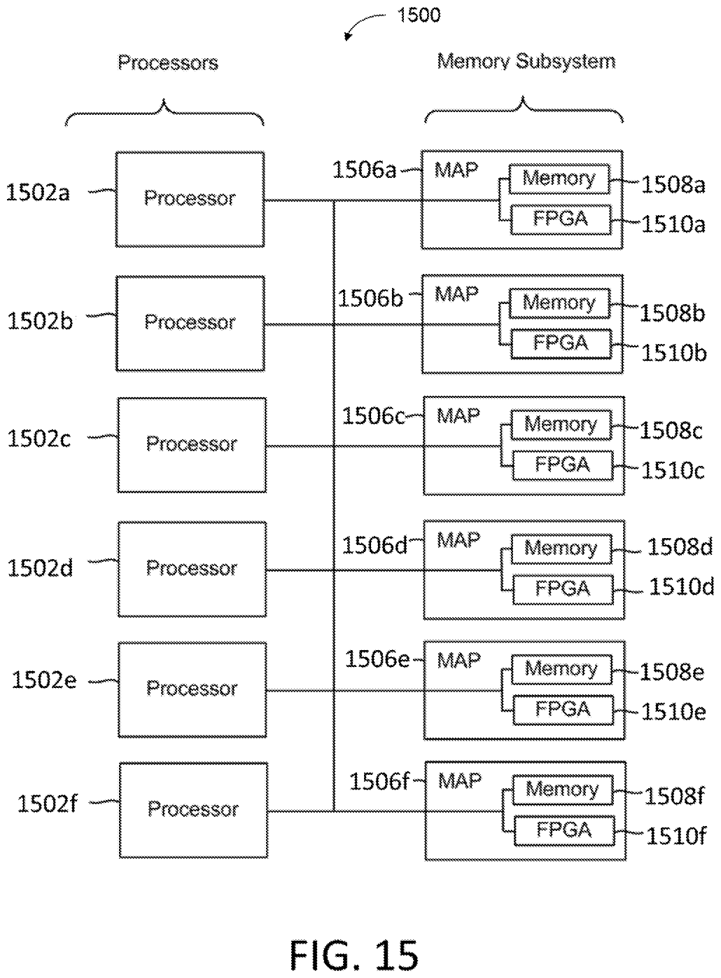

FIG. 15 is a block diagram of a multiprocessor computer system 1500 using a shared virtual address memory space in accordance with an example instance. The system includes a plurality of processors 1502a-f that can access a shared memory subsystem 1504. The system incorporates a plurality of programmable hardware memory algorithm processors (MAPs) 1506a-f in the memory subsystem 1504. Each MAP 1506a-f can comprise a memory 1508a-f and one or more field programmable gate arrays (FPGAs) 1510a-f. The MAP provides a configurable functional unit and particular algorithms or portions of algorithms can be provided to the FPGAs 1510a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1508a-f, allowing it to execute tasks independently of, and asynchronously from the respective microprocessor 1502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 15, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 13.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with DI water, held under a DI water gooseneck faucet for 5 minutes, and dried with N₂. The substrate was subsequently soaked in NH40H (1:100; 3 mL:300 mL) for 5 minutes, rinsed with DI water using a handgun, soaked in three successive beakers with DI water for 1 minute each, and then rinsed again with DI water using the handgun. The substrate was then plasma cleaned by exposing the substrate surface to O₂. A SAMCO PC-300 instrument was used to plasma etch O₂ at 250 watts for 1 minute in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 minutes, 70° C., 135° C. vaporizer. The substrate surface was resist coated using a Brewer Science 200× spin coater. SPR™ 3612 photoresist was spin coated on the substrate at 2500 rpm for 40 seconds. The substrate was pre-baked for 30 minutes at 90° C. on a Brewer hot plate. The substrate was subjected to photolithography using a Karl Suss MA6 mask aligner instrument. The substrate was exposed for 2.2 seconds and developed for 1 minute in MSF 26A. Remaining developer was rinsed with the handgun and the substrate soaked in water for 5 minutes. The substrate was baked for 30 minutes at 100° C. in the oven, followed by visual inspection for lithography defects using a Nikon L200. A descum process was used to remove residual resist using the SAMCO PC-300 instrument to O₂ plasma etch at 250 watts for 1 minute.

The substrate surface was passively functionalized with a 100 μL solution of perfluorooctyltrichlorosilane mixed with 10 μL light mineral oil. The substrate was placed in a chamber, pumped for 10 minutes, and then the valve was closed to the pump and left to stand for 10 minutes. The chamber was vented to air. The substrate was resist stripped by performing two soaks for 5 minutes in 500 mL NMP at 70° C. with ultrasonication at maximum power (9 on Crest system). The substrate was then soaked for 5 minutes in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-Mer Sequence on a Polynucleotide Synthesis Device

A two dimensional polynucleotide synthesis device was assembled into a flowcell, which was connected to a flowcell (Applied Biosystems (ABI394 DNA Synthesizer”). The polynucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1. 5′AGACAATCAACCATTTGGGGTGGACAGCCTTGACCTCTAGACTTCGGCAT##TTTTTTTTT T3′ (SEQ ID NO.: 1), where # denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 2 and an ABI synthesizer.

TABLE 2

| | Table 2 | |
|---|---|---|
| General DNA Synthesis | | Time |
| Process Name | Process Step | (seconds) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| | Acetonitrile System Flush | 4 |
| WASH (Acetonitrile Wash | Acetonitrile to Flowcell | 15 |
| Flow) | N2 System Flush | 4 |
| | Acetonitrile System Flush | 4 |
| CAPPING (CapA+B, 1:1, | CapA+B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I₂ in 20% pyridine, 10% water, and 70% THF) were roughly ˜100 uL/second, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/second, and for Deblock (3% dichloroacetic acid in toluene), roughly ˜300 uL/second (compared to ˜50 uL/second for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence on a Polynucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATGCT AGCCATACCATGATGATGATGATGATGAGAACCCCGCAT##TTTTTTTTTT3′, where # denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 master mix, 2.5 uL 10 uM Forward primer, 2.5 uL 10 uM Reverse primer, 1 uL polynucleotide extracted from the surface, and water up to 50 uL) using the following thermal cycling program:

98 C, 30 seconds

98 C, 10 seconds; 63 C, 10 seconds; 72 C, 10 seconds; repeat 12 cycles

72 C, 2 minutes

The PCR products were also run on a BioAnalyzer (data not shown), demonstrating sharp peaks at the 100-mer position. Next, the PCR amplified samples were cloned, and Sanger sequenced. Table 3 summarizes the results from the Sanger sequencing for samples taken from spots 1-5 from chip 1 and for samples taken from spots 6-10 from chip 2.

TABLE 3

| Spot | Error rate | | Cycle efficiency |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors.

Finally, Table 4 summarizes error characteristics for the sequences obtained from the polynucleotides samples from spots 1-10.

TABLE 4

| Sample | | | | | | | | | | |
|---|---|---|---|---|---|---|---|---|---|---|
| ID/Spot | OSA_0046/ | OSA_0047/ | OSA_0048/ | OSA_0049/ | OSA_0050/ | OSA_0051/ | OSA_0052/ | OSA_0053/ | OSA_0054/ | OSA_0055/ |
| no. | 1 | 2 | 3 | 4 | 5 | 6 | 7 | 8 | 9 | 10 |
| Total | 32 | 32 | 32 | 32 | 32 | 32 | 32 | 32 | 32 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI Match | 2500 | 2698 | 2561 | 2122 | 2499 | 2666 | 2625 | 2899 | 2798 | 2348 |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI Single | 0 | 0 | 0 | 0 | 0 | 0 | 0 | 0 | 0 | 0 |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI Minus | MP Err: | MP Err: | MP Err: | MP Err: | MP Err: | MP Err: | MP Err: | MP Err: | MP Err: | MP Err: |
| Primer | ~1 in | ~1 in | ~1 in | ~1 in | ~1 in | ~1 in | ~1 in | ~1 in | ~1 in | ~1 in |
| Error Rate | 763 | 824 | 780 | 429 | 1525 | 1615 | 531 | 1769 | 854 | 1451 |

### Example 4: Parallel Assembly of 29,040 Unique Polynucleotides

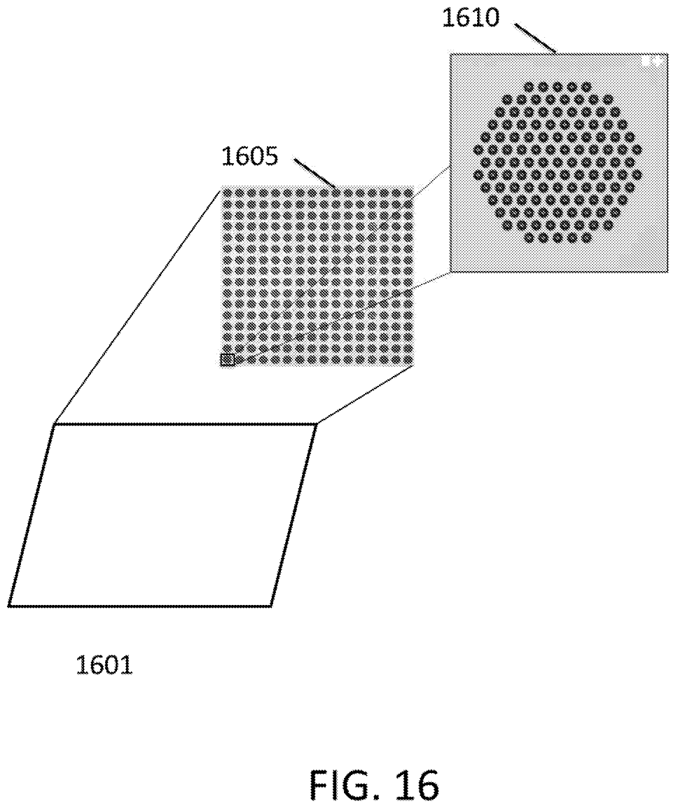

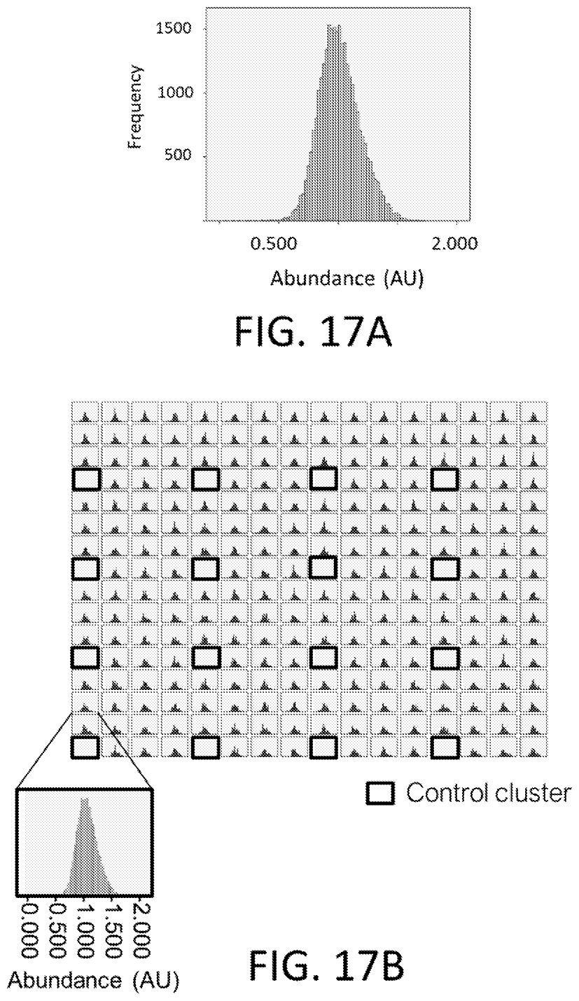

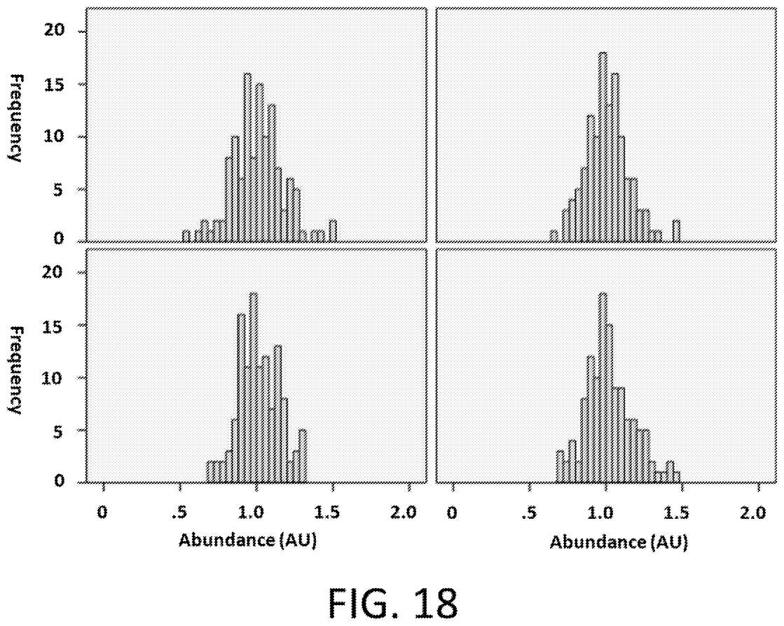

A structure comprising 256 clusters 1605 each comprising 121 loci on a flat silicon plate 1601 was manufactured as shown in FIG. 16. An expanded view of a cluster is shown in 1610 with 121 loci. Loci from 240 of the 256 clusters provided an attachment and support for the synthesis of polynucleotides having distinct sequences. Polynucleotide synthesis was performed by phosphoramidite chemistry using general methods from Example 3. Loci from 16 of the 256 clusters were control clusters. The global distribution of the 29,040 unique polynucleotides synthesized (240×121) is shown in FIG. 17A. Polynucleotide libraries were synthesized at high uniformity. 90% of sequences were present at signals within 4× of the mean, allowing for 100% representation. Distribution was measured for each cluster, as shown in FIG. 17B. The distribution of unique polynucleotides synthesized in 4 representative clusters is shown in FIG. 18. On a global level, all polynucleotides in the run were present and 99% of the polynucleotides had abundance that was within 2× of the mean indicating synthesis uniformity. This same observation was consistent on a per-cluster level.

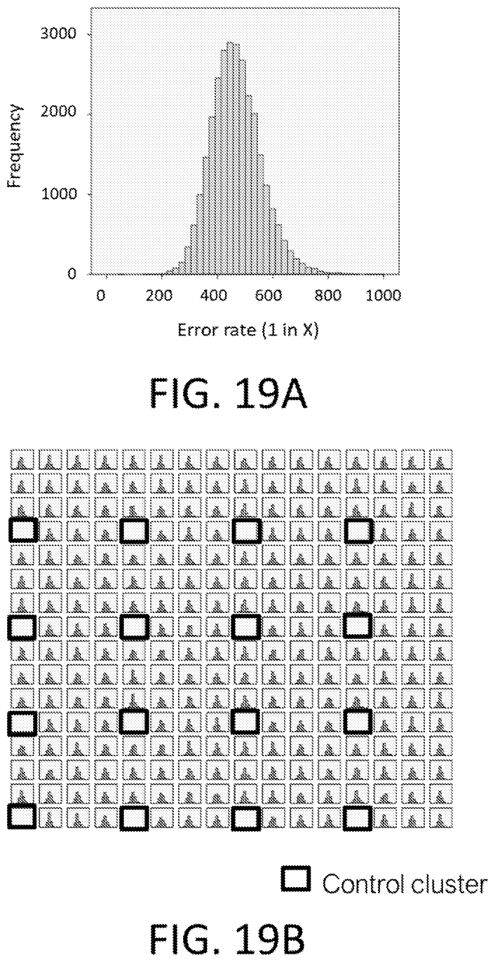

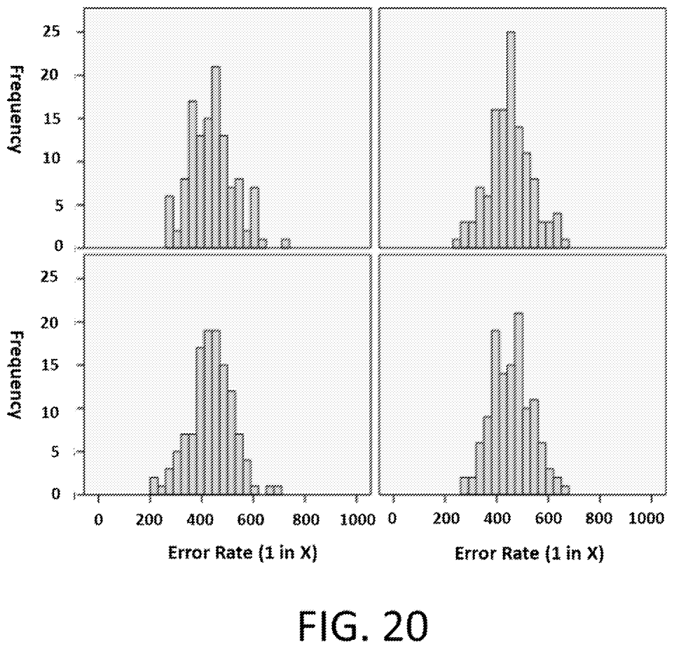

The error rate for each polynucleotide was determined using an Illumina MiSeq gene sequencer. The error rate distribution for the 29,040 unique polynucleotides is shown in FIG. 19A and averages around 1 in 500 bases, with some error rates as low as 1 in 800 bases. Distribution was measured for each cluster, as shown in FIG. 19B. The error rate distribution for unique polynucleotides in four representative clusters is shown in FIG. 20. The library of 29,040 unique polynucleotides was synthesized in less than 20 hours.

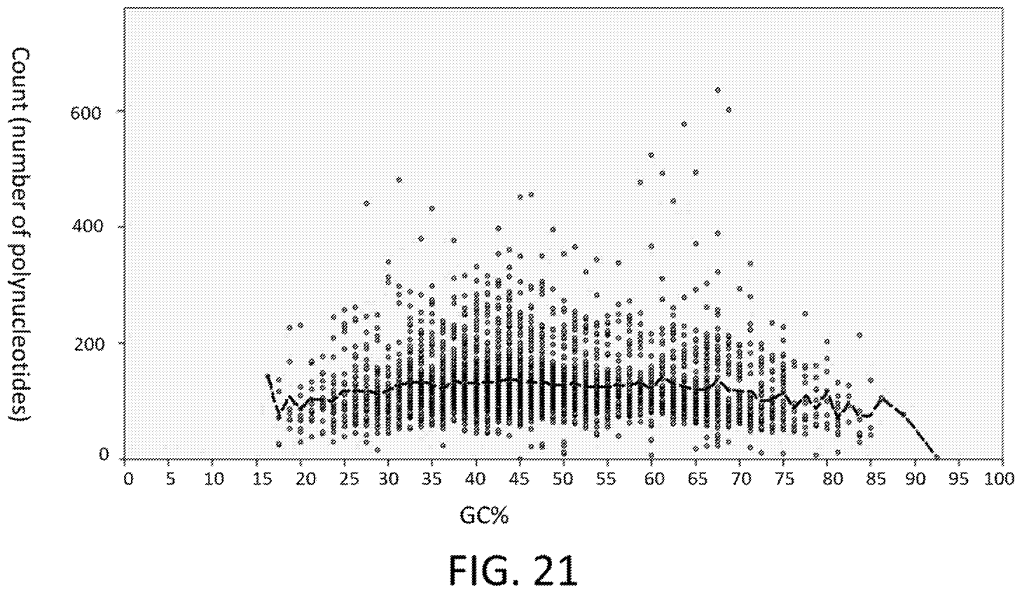

Analysis of GC percentage versus polynucleotide representation across all of the 29,040 unique polynucleotides showed that synthesis was uniform despite GC content, FIG. 21.

### Example 5: Sample Preparation and Enrichment with a Polynucleotide Targeting Library

Genomic DNA (gDNA) was obtained from a sample, and fragmented enzymatically in a fragmentation buffer, end-repaired, and 3′ adenylated. Dual-index adapters (16 unique barcode combinations) were ligated to both ends of the genomic DNA fragments to produce a library of adapter-tagged gDNA strands, and the adapter-tagged DNA library is amplified with a high-fidelity polymerase. The gDNA library was then denatured into single strands at 96° C., in the presence of universal adapter blockers. A polynucleotide targeting library (probe library) was denatured in a hybridization solution at 96° C., and combined with the denatured, tagged gDNA library in hybridization solution for 16 hours at 70° C. Binding buffer was then added to the hybridized tagged gDNA-probes, and magnetic beads comprising streptavidin were used to capture biotinylated probes. The beads were separated from the solution using a magnet, and the beads were washed three times with buffer to remove unbound adapters, gDNA, and adapter blockers before an elution buffer was added to release the enriched, tagged gDNA fragments from the beads. The enriched library of tagged gDNA fragments was amplified with a high-fidelity polymerase to get yields sufficient for cluster generation, and then the library was sequenced using an NGS instrument.

### Example 6: Genomic DNA Capture with an Exome-Targeting Polynucleotide Probe Library

A polynucleotide targeting library comprising at least 500,000 non-identical polynucleotides targeting the human exome was designed and synthesized on a structure by phosphoramidite chemistry using the general methods from Example 3, and the stoichiometry controlled using the general methods of Example 5 to generate Library 4. The polynucleotides were then labeled with biotin, and then dissolved to form an exome probe library solution. A dried indexed library pool was obtained from a genomic DNA (gDNA) sample using the general methods of Example 16.

The exome probe library solution, a hybridization solution, a blocker mix A, and a blocker mix B were mixed by pulse vortexing for 2 seconds. The hybridization solution was heated at 65° C. for 10 minutes, or until all precipitate was dissolved, and then brought to room temperature on the benchtop for 5 additional minutes. 20 μL of hybridization solution and 4 μL of the exome probe library solution were added to a thin-walled PCR 0.2 mL strip-tube and mixed gently by pipetting. The combined hybridization solution/exome probe solution was heated to 95° C. for 2 minutes in a thermal cycler with a 105° C. lid and immediately cooled on ice for at least 10 minutes. The solution was then allowed to cool to room temperature on the benchtop for 5 minutes. While the hybridization solution/exome probe library solution was cooling, water was added to 9 μl for each genomic DNA sample, and 5 μL of blocker mix A, and 2 μL of blocker mix B were added to the dried indexed library pool in the thin-walled PCR 0.2 mL strip-tube. The solution was then mixed by gentle pipetting. The pooled library/blocker tube was heated at 95° C. for 5 minutes in a thermal cycler with a 105° C. lid, then brought to room temperature on the benchtop for no more than 5 minutes before proceeding onto the next step. The hybridization mix/probe solution was mixed by pipetting and added to the entire 24 μL of the pooled library/blocker tube. The entire capture reaction well was mixed by gentle pipetting, to avoid generating bubbles. The sample tube was pulse-spun to make sure the tube was sealed tightly. The capture/hybridization reaction was heated at 70° C. for 16 hours in a PCR thermocycler, with a lid temperature of 85° C.

Binding buffer, wash Buffer 1 and wash Buffer 2 were heated at 48° C. until all precipitate was dissolved into solution. 700 μL of wash buffer 2 was aliquoted per capture and preheated to 48° C. Streptavidin binding beads and DNA purification beads were equilibrated at room temperature for at least 30 minutes. A polymerase, such as KAPA HiFi HotStart ReadyMix and amplification primers were thawed on ice. Once the reagents were thawed, they were mixed by pulse vortexing for 2 seconds. 500 μL of 80 percent ethanol per capture reaction was prepared. Streptavidin binding beads were pre-equilibrated at room temperature and vortexed until homogenized. 100 μL of streptavidin binding beads were added to a clean 1.5 mL microcentrifuge tube per capture reaction. 200 μL of binding buffer was added to each tube and each tube was mixed by pipetting until homogenized. The tube was placed on magnetic stand. Streptavidin binding beads were pelleted within 1 minute. The tube was removed and the clear supernatant was discarded, making sure not to disturb the bead pellet. The tube was removed from the magnetic stand, and the washes were repeated two additional times. After the third wash, the tube was removed and the clear supernatant was discarded. A final 200 μL of binding buffer was added, and beads were resuspended by vortexing until homogeneous.

After completing the hybridization reaction, the thermal cycler lid was opened and the full volume of capture reaction was quickly transferred (36-40 μL) into the washed streptavidin binding beads. The mixture was mixed for 30 minutes at room temperature on a shaker, rocker, or rotator at a speed sufficient to keep capture reaction/streptavidin binding bead solution homogenized. The capture reaction/streptavidin binding bead solution was removed from mixer and pulse-spun to ensure all solution was at the bottom of the tube. The sample was placed on a magnetic stand, and streptavidin binding beads pelleted, leaving a clear supernatant within 1 minute. The clear supernatant was removed and discarded. The tube was removed from the magnetic stand and 200 μL of wash buffer was added at room temperature, followed by mixing by pipetting until homogenized. The tube was pulse-spun to ensure all solution was at the bottom of the tube. A thermal cycler was programmed with the following conditions (Table 5).

The temperature of the heated lid was set to 105° C.

TABLE 5

| Step | Temperature | Time | | Cycle Number |
|---|---|---|---|---|
| 1 | 98° C. | 45 | seconds | 1 |
| 2 | 98° C. | 15 | seconds | 9 |
| | 60° C. | 30 | seconds | |
| | 72° C. | 30 | seconds | |
| 3 | 72° C. | 1 | minute | 1 |

| | | |
|---|---|---|
| 4 | 4° C. | HOLD |

Amplification primers (2.5 μL) and a polymerase, such as KAPA HiFi HotStart ReadyMix (25 μL) were added to a tube containing the water/streptavidin binding bead slurry, and the tube mixed by pipetting. The tube was then split into two reactions. The tube was pulse-spun and transferred to the thermal cycler and the cycling program in Table 5 was started. When thermal cycler program was complete, samples were removed from the block and immediately subjected to purification. DNA purification beads pre-equilibrated at room temperature were vortexed until homogenized. 90 μL (1.8×) homogenized DNA purification beads were added to the tube, and mixed well by vortexing. The tube was incubated for 5 minutes at room temperature, and placed on a magnetic stand. DNA purification beads pelleted, leaving a clear supernatant within 1 minute. The clear supernatant was discarded, and the tube was left on the magnetic stand. The DNA purification bead pellet was washed with 200 μL of freshly prepared 80 percent ethanol, incubated for 1 minute, then removed and the ethanol discarded. The wash was repeated once, for a total of two washes, while keeping the tube on the magnetic stand. All remaining ethanol was removed and discarded with a 10 μL pipette, making sure to not disturb the DNA purification bead pellet. The DNA purification bead pellet was air-dried on a magnetic stand for 5-10 minutes or until the pellet was dry. The tube was removed from the magnetic stand and 32 μL of water was added, mixed by pipetting until homogenized, and incubated at room temperature for 2 minutes. The tube was placed on a magnetic stand for 3 minutes or until beads were fully pelleted. 30 μL of clear supernatant was recovered and transferred to a clean thin-walled PCR 0.2 mL strip-tube, making sure not to disturb DNA purification bead pellet. Average fragment length was between about 375 bp to about 425 bp using a range setting of 150 bp to 1000 bp on an analysis instrument. Ideally, the final concentration values is at least about 15 ng/μL. Each capture was quantified and validated using Next Generation Sequencing (NGS).

A summary of NGS metrics is shown in Table 6, Table 7 as compared to a comparator exome capture kit (Comparator Kit D). Library 4 has probes (baits) that correspond to a higher percentage of exon targets than Comparator Kit D. This results in less sequencing to obtain comparable quality and coverage of target sequences using Library 4.

TABLE 6

| NGS Metric | Comparator Kit D | | Library 4 | |
|---|---|---|---|---|
| Target Territory | 38.8 | Mb | 33.2 | Mb |
| Bait Territory | 50.8 | Mb | 36.7 | Mb |
| Bait Design Efficiency | 76.5% | | 90.3% | |
| Capture Plex | 8-plex | | 8-plex | |
| PF Reads | 57.7 | M | 49.3 | M |
| Normalized Coverage | 150 | X | 150 | X |
| HS Library Size | 30.3 | M | 404.0 | M |

| | | |
|---|---|---|
| Percent Duplication | 32.5% | 2.5% |
| Fold Enrichment | 43.2 | 48.6 |
| Fold 80 Base Penalty | 1.84 | 1.40 |

TABLE 7

| NGS Metric | Comparator Kit D | Library 4 |
|---|---|---|
| Percent Pass Filtered Unique Reads | 67.6% | 97.5% |
| (PCT_PF_UQ_READS) | | |
| Percent Target Bases at 1X | 99.8% | 99.8% |
| Percent Target Bases at 20X | 90.3% | 99.3% |
| Percent Target Bases at 30X | 72.4% | 96.2% |

A comparison of overlapping target regions for both Kit D and Library 4 (total reads normalized to 96× coverage) is shown in Table 8. Library 4 was processed as 8 samples per hybridization, and Kit D was processed at 2 samples per hybridization. Additionally, for both libraries, single nucleotide polymorphism and in-frame deletion calls from overlapping regions were compared against high-confidence regions identified from “Genome in a Bottle” NA12878 reference data (Table 9). Library 4 performed similarly or better (higher indel precision) that Kit D in identifying SNPs and indels. The term “indel(s)” as used herein refers a type of error inclusive of insertions and deletions that differ from a predetermined sequence.

TABLE 8

| NGS Metric | Comparator Kit D | | Library 4 | |
|---|---|---|---|---|
| Percent Pass Filtered Reads | 94.60% | | 97.7% | |
| (PCT_PF_UQ_READS) | | | | |
| Percent Selected Bases | 79% | | 80% | |
| Percent Target Bases at 1X | 100% | | 100% | |
| Percent Target Bases at 20X | 90% | | 96% | |
| Percent Target Bases at 30X | 71% | | 77% | |
| Fold Enrichment | 44.9 | | 49.9 | |
| Fold 80 Base Penalty | 1.76 | | 1.4 | |
| HS Library Size | 122 | M | 267 | M |

TABLE 9

| | Comparator Kit D | | Library 4 | |
|---|---|---|---|---|
| Variants | Precision | Sensitivity | Precision | Sensitivity |
| Single Nucleotide | 98.59% | 99.23% | 99.05% | 99.27% |
| Polymorphisms (SNPs) | | | | |
| In-Frame Deletions | 76.42% | 94.12% | 87.76% | 94.85% |
| (Indels) | | | | |
| Total | 98.14% | 99.15% | 98.85% | 99.20% |

Precision represents the ratio of true positive calls to total (true and false) positive calls. Sensitivity represents the ratio of true positive calls to total true values (true positive and false negative).

### Example 7. Library Preparation with Universal Adapters

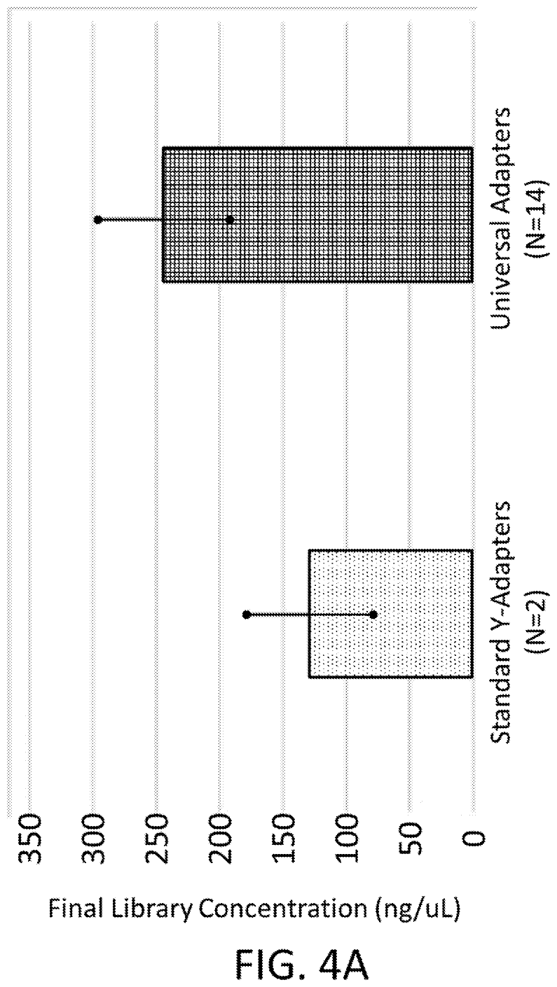

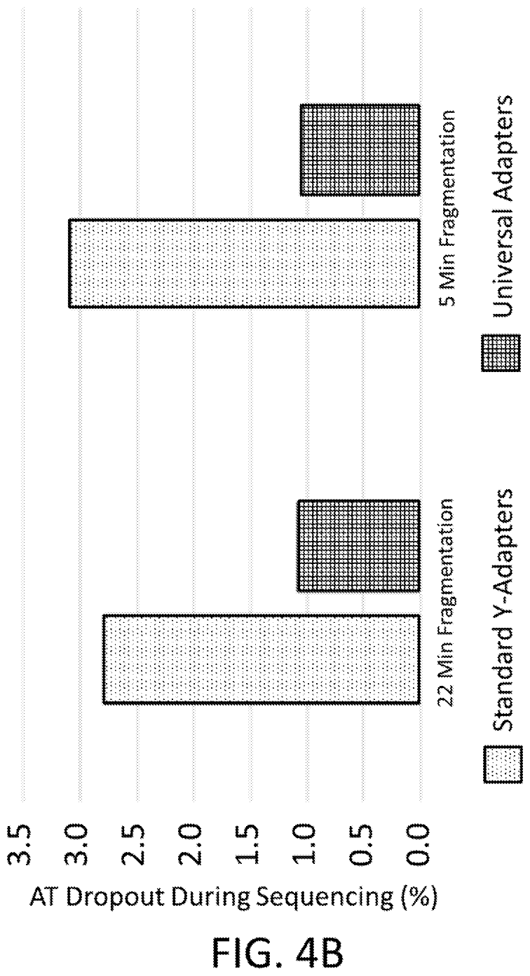

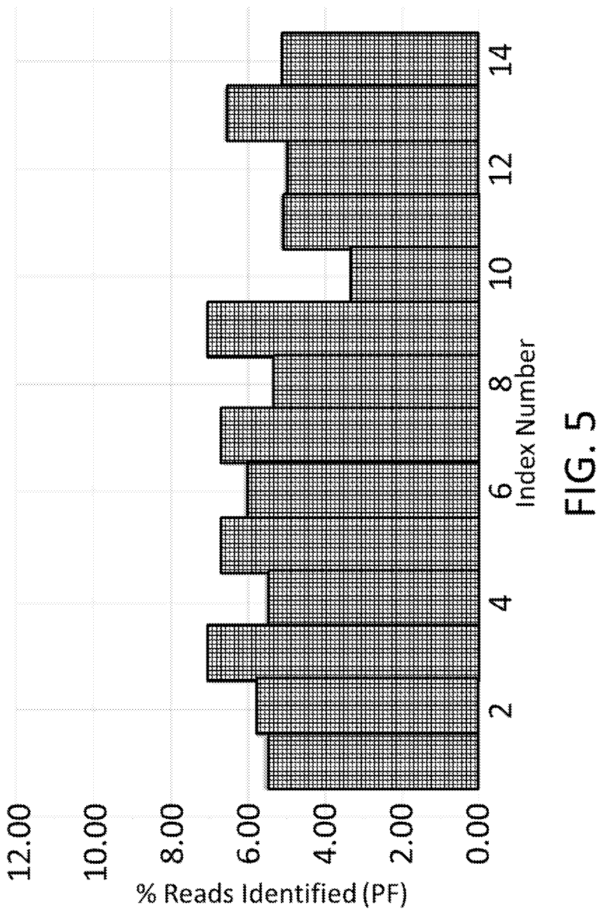

A nucleic acid sample was prepared using the general methods of example 5 or 6, with modification: dual-index adapters were replaced with universal adapters. After ligation of universal adapters, amplification of the adapter-ligated sample nucleic acid library was conducted with a barcoded primer library, to generate a barcoded adapter-ligated sample nucleic acid library. This library was then sequenced directly. Use of universal adapters resulted in increased library nucleic acid concentration after amplification (FIG. 4A) relative to standard dual-index Y-adapters. Additionally, a library prepared with universal adapters provided for lower AT dropouts compared to standard dual-index Y-adapters, (FIG. 4B), and resulted in uniform representation of all index sequences. (FIG. 5)

### Example 8. Library Preparation with Universal Adapters and Enrichment

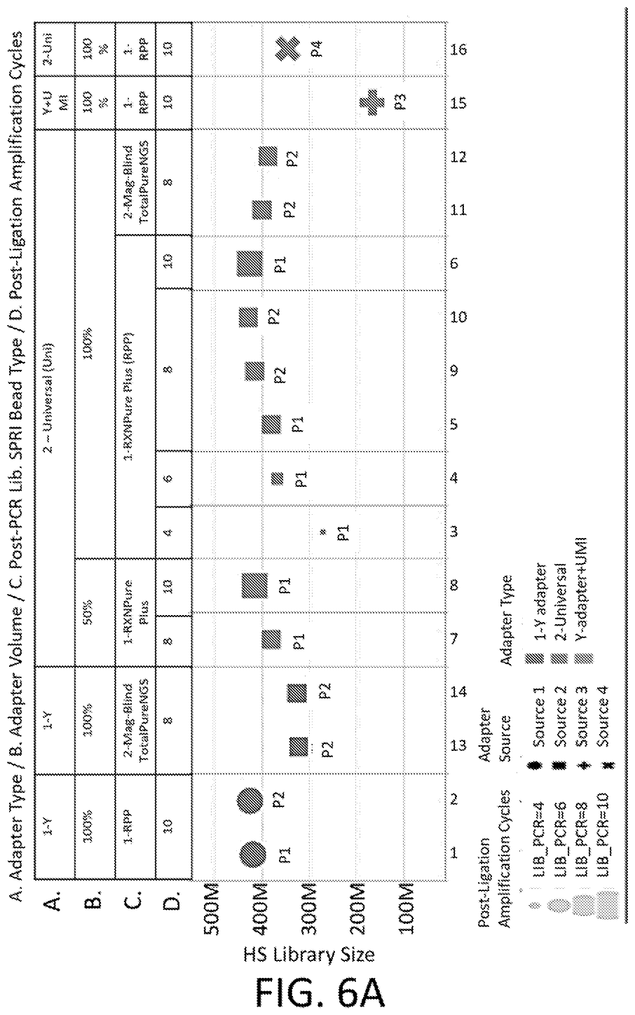

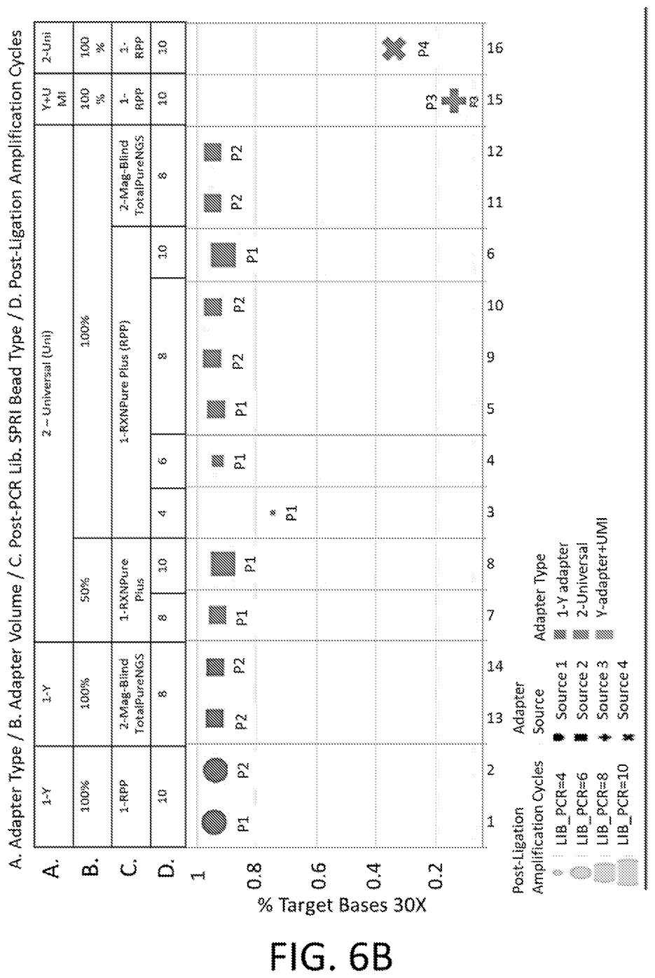

A nucleic acid sample was prepared using the general methods of Example 5 or 6, with modification: dual-index adapters were replaced with universal adapters. After ligation of universal adapters, amplification of the adapter-ligated sample nucleic acid library was conducted with a barcoded primer library, to generate a barcoded adapter-ligated sample nucleic acid library. This library was then subjected to analogous enrichment, purification, and sequencing steps. Use of universal adapters resulted in comparable or better sequencing outcomes (FIG. 6A and FIG. 6B).

### Example 9. Library Preparation with Universal Adapters Comprising Modified Bases

A nucleic acid sample is prepared using the general methods of Example 8, with modification: universal adapters comprise at least one locked nucleic acid or bridged nucleic acid. After ligation of universal adapters, amplification of the adapter-ligated sample nucleic acid library is conducted with a barcoded primer library, to generate a barcoded adapter-ligated sample nucleic acid library. This library is then subjected to analogous enrichment, purification, and sequencing steps.

### Example 10. Library Preparation with Universal Adapters with Short Barcoded Primers

A nucleic acid sample is prepared using the general methods of Example 8, with modification: Each of the barcoded primers binds to less than the entire length of the universal adapter.

### Example 11. Library Preparation with Nucleobase Analogue-Containing Universal Adapters and Amplification with Short Barcoded Primers

A nucleic acid sample is prepared using the general methods of Example 8, with modification: dual-index adapters are replaced with universal adapters comprising one or more nucleobase analogue (e.g., locked nucleic acid or bridged nucleic acid). After ligation of universal adapters, amplification of the adapter-ligated sample nucleic acid library is conducted with a barcoded primer library, to generate a barcoded adapter-ligated sample nucleic acid library. Each of the barcodes binds to less than the entire length of the universal adapter. This library is then subjected to analogous enrichment, purification, and sequencing steps.

### Example 12. Comparison of Sequencing Libraries Prepared with Universal Adapters and Standard Dual-Index Adapters

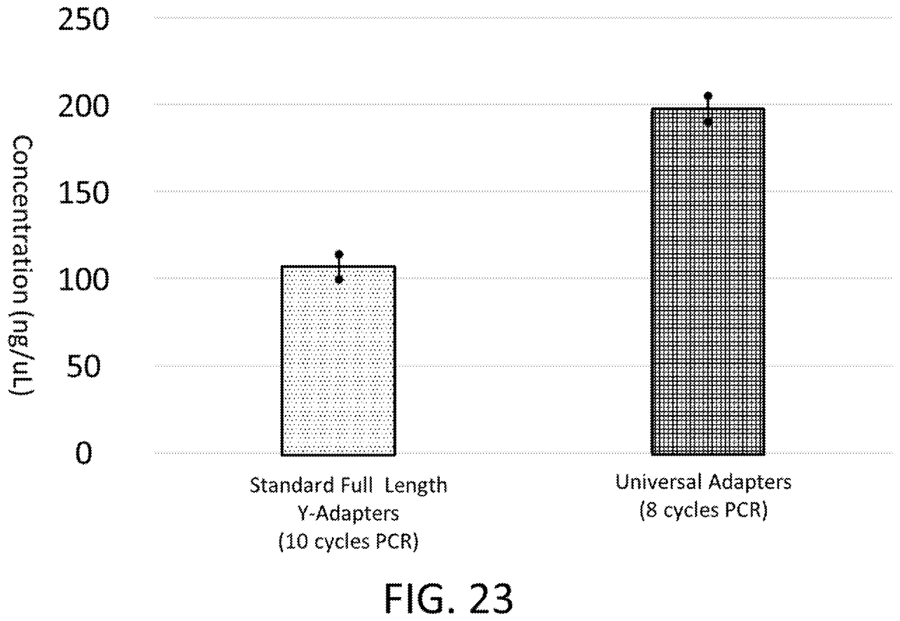

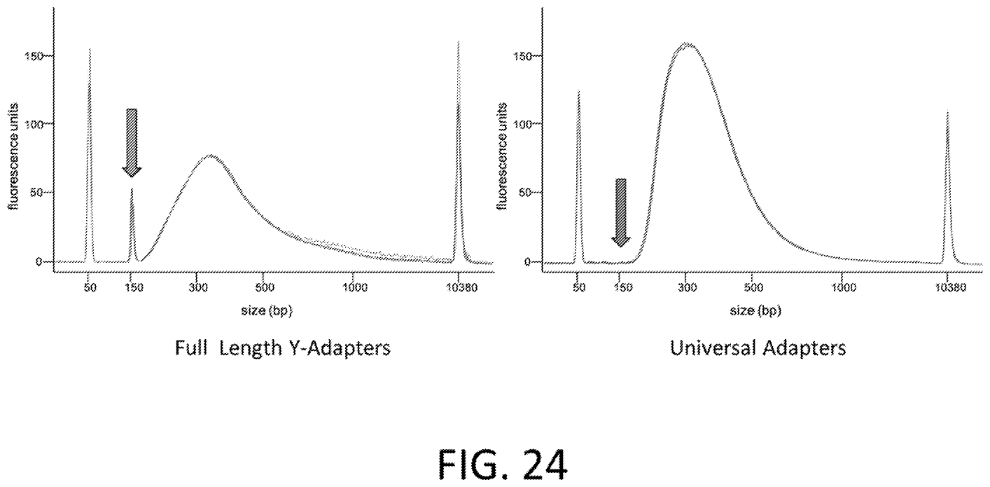

A nucleic acid sample was prepared from genomic DNA (50 ng of NA12878) using the general methods of Example 8, with modification: universal adapters comprising 10 bp dual indices were utilized (8 PCR cycles, N=12). For comparison, standard full-length Y adapters were also tested for the same genomic DNA sample (10 PCR cycles, N=12). The protocol utilizing universal adapters led to higher total yields after amplification (FIG. 23), and lower adapter dimer formation (FIG. 24).

### Example 13. Comparison of Sequencing Libraries Prepared with 10 bp UDI Universal Adapters and 8 bp Combinatorial Dual Primers

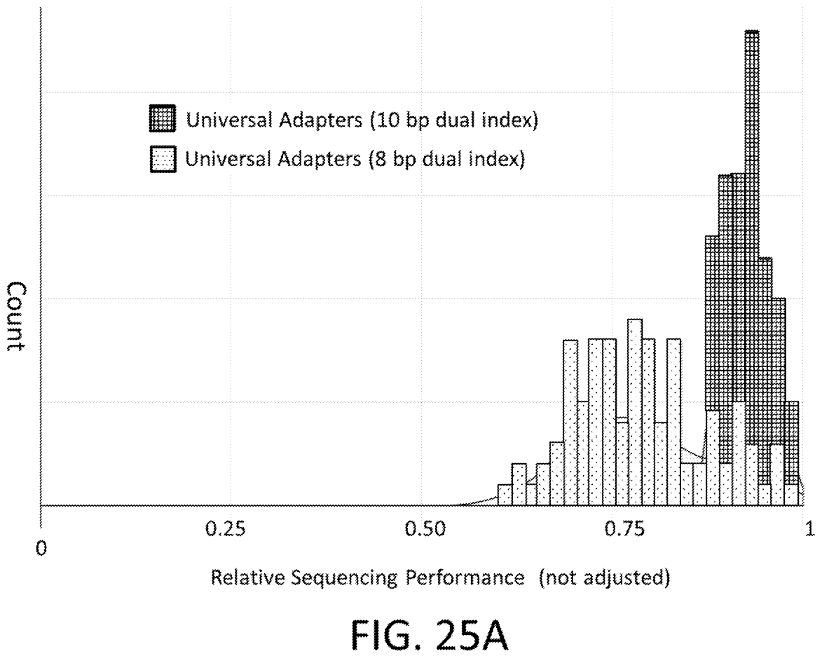

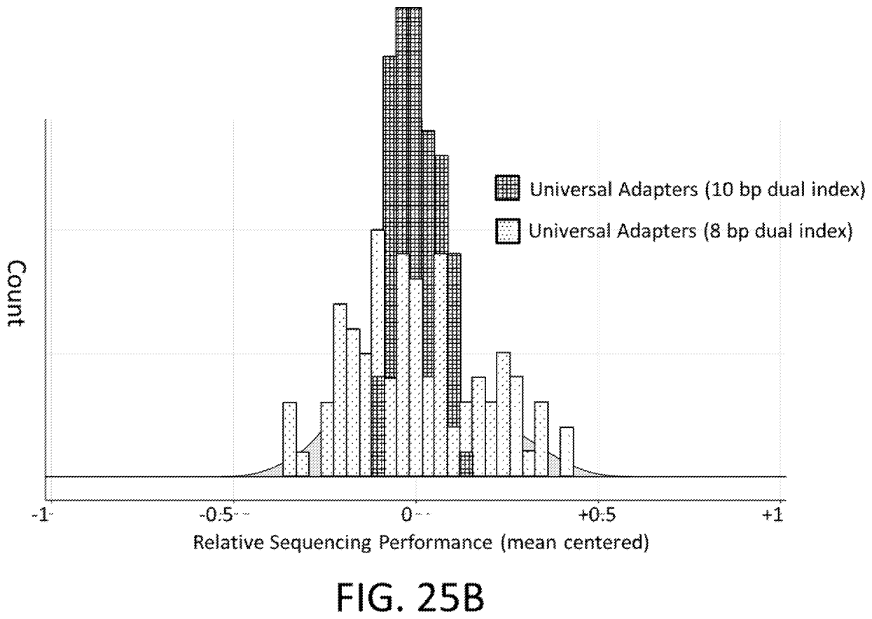

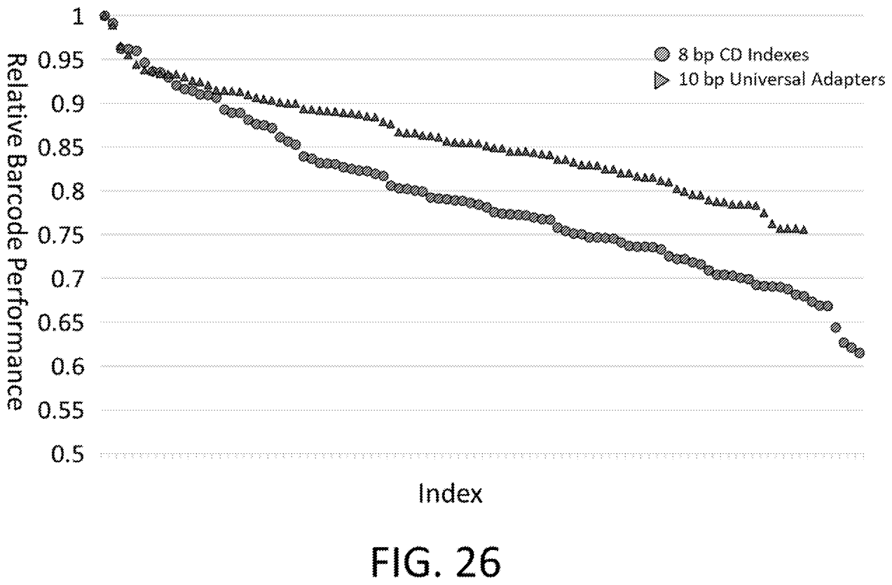

A nucleic acid sample was prepared from genomic DNA (NA12878) using the general methods of Example 8, with modification: universal primers comprising either a 10 bp index sequence (N=96) or an 8 bp index sequence (N=96) were used for the final amplification step of the library. Relative sequencing performance was calculated by normalizing the total number of perfect index reads for each design and normalizing to the top performer; resulting distributions of each population were centered on their calculated mean for direct comparison. The experiment using 10 bp universal primers exhibited a tighter relative performance and more even sequencing representation (FIGS. 25A and 25B) and had higher relative performance across all 96 unique indexes (FIG. 26).

### Example 14. Screening and Evaluation of Unique Dual Index Libraries

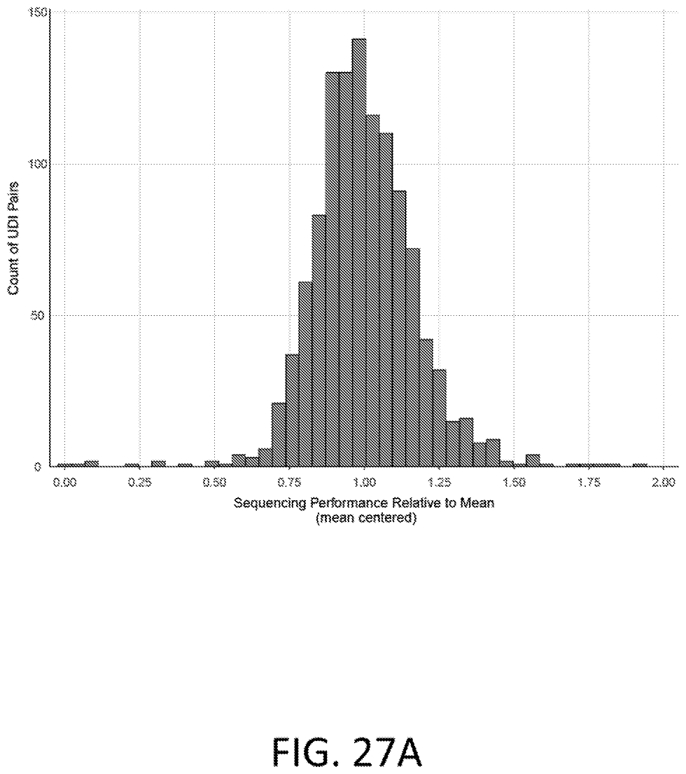

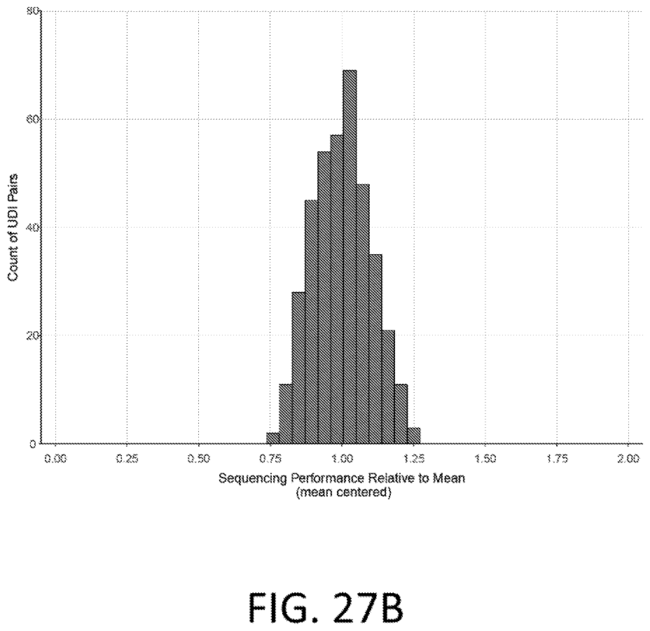

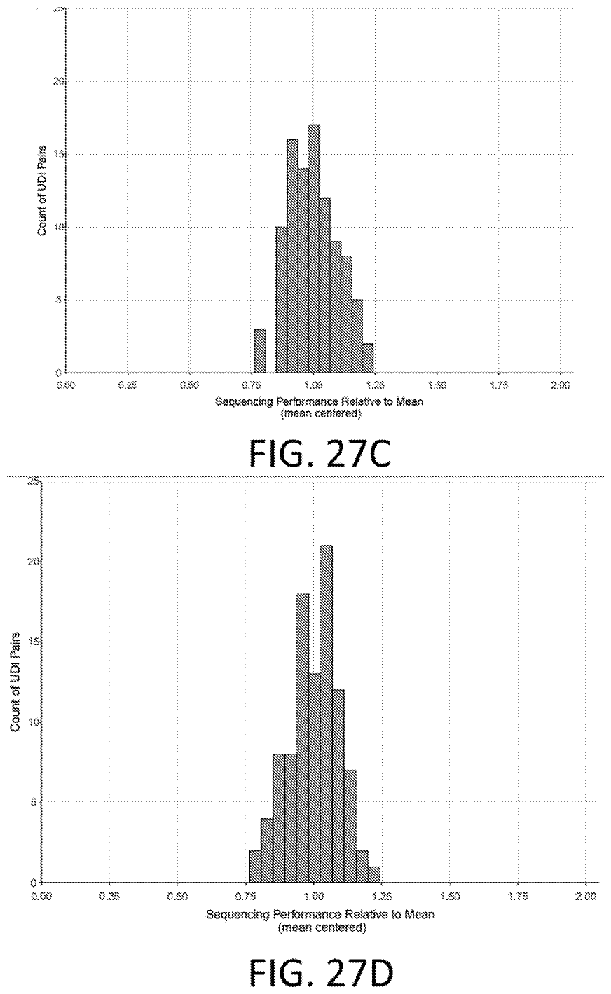

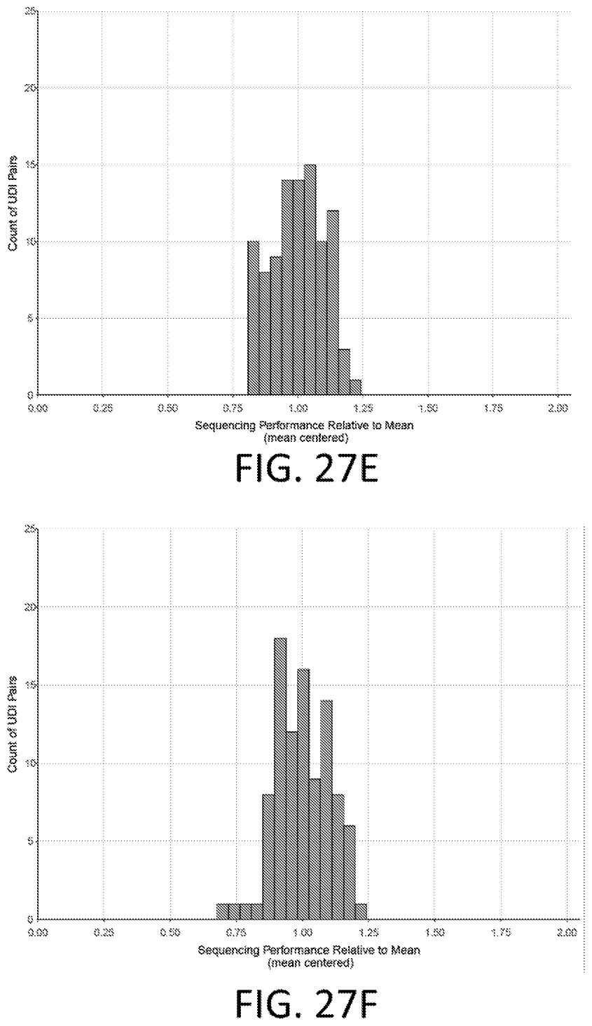

Following the general procedures of Example 13, 1,152 libraries containing unique dual index sequences were constructed and screened in an iterative fashion for even sequencing performance (FIG. 27A). Libraries were generated using enzymatic fragmentation and comprised human genomic material as an insert. Individual libraries were pooled by mass and sequenced with a NextSeq 500/550 High Output v2 kit to generate 2×10 bp index reads. The total count of individual pairs of index reads (1 mismatch allowed) was determined and the relative performance of each individual pair was calculated relative to the mean. As a result, 384 UDI sequences were identified that provided sequencing performance relative to the mean of +/−25% either as a single large pool (FIG. 27B) or as individual sets of 4×96 members (FIGS. 27C-27F).

### Example 15: Genomic DNA Capture with Various Exome-Targeting Polynucleotide Probe Libraries

A polynucleotide targeting library comprising at least 500,000 non-identical polynucleotides targeting the human exome was designed and synthesized on a structure by phosphoramidite chemistry using the general methods from Example 3, and the stoichiometry controlled using the general methods of Example 5 to generate Library 4A. The polynucleotides were then labeled with biotin, and then dissolved to form an exome probe library solution. A dried indexed library pool was obtained from a genomic DNA (gDNA) sample using the general methods of Example 5.

DNA capture using the various probe libraries was performed using method as described in Example 6. Briefly, the exome probe library solution, a hybridization solution, a blocker mix A, and a blocker mix B were mixed, and hybridization mix/probe solution prepared. A hybridization reaction was performed followed by a capture reaction. The solution was then subject to amplification followed by Next Generation Sequencing (NGS).

Library 4A was compared to various comparator exome capture kits including Comparator Kit D described in Example 6. A summary of NGS metrics is shown in Table 10 of the various comparator exome capture kits with Library 4A.

TABLE 10

| | Comparator | | Comparator | | Comparator | | Library | |
|---|---|---|---|---|---|---|---|---|
| | A1 | | A2 | | D | | 4A | |
| Target Size | 38.0 | Mb | 35.8 | Mb | 38.8 | Mb | 33.1 | Mb |
| Design Size | 60.5 | Mb | 49.5 | Mb | 50.8 | Mb | 36.6 | Mb |

| | | | | |
|---|---|---|---|---|
| % Probe | 62.9% | 72.4% | 76.5% | 90.3% |
| Design Efficiency | | | | |
| % of Bases | 37.1% | 27.6% | 23.5% | 9.7% |
| Outside of | | | | |
| Target Region | | | | |

Library Prep

| | | | | | | | | |
|---|---|---|---|---|---|---|---|---|
| Input DNA | 1000 | ng | 1000 | ng | 100 | ng | 50 | ng |
| Input Library | 750 | ng | 750 | ng | 500 | ng | 187.5 | ng |

| | | | | |
|---|---|---|---|---|
| Plex | Single | Single | 8-plex | 8-plex |

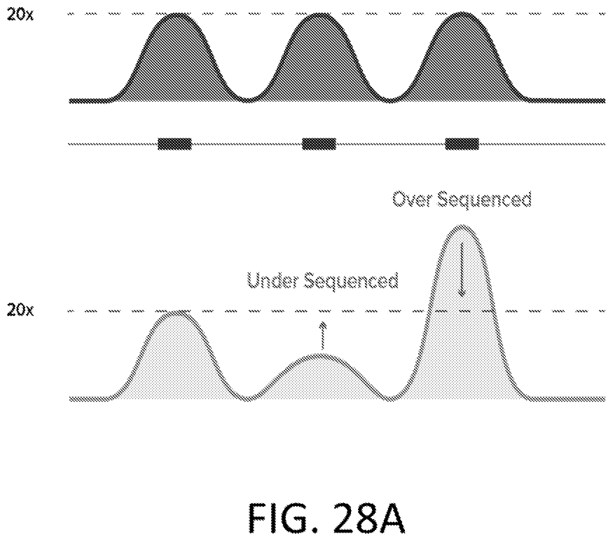

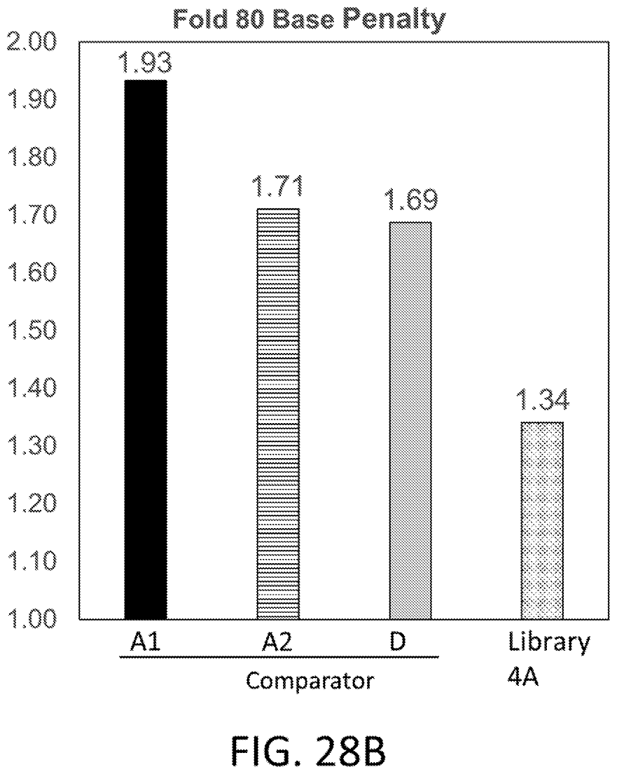

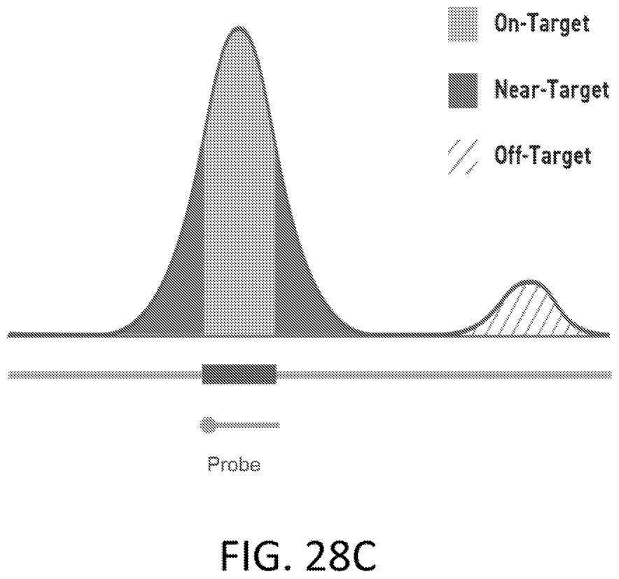

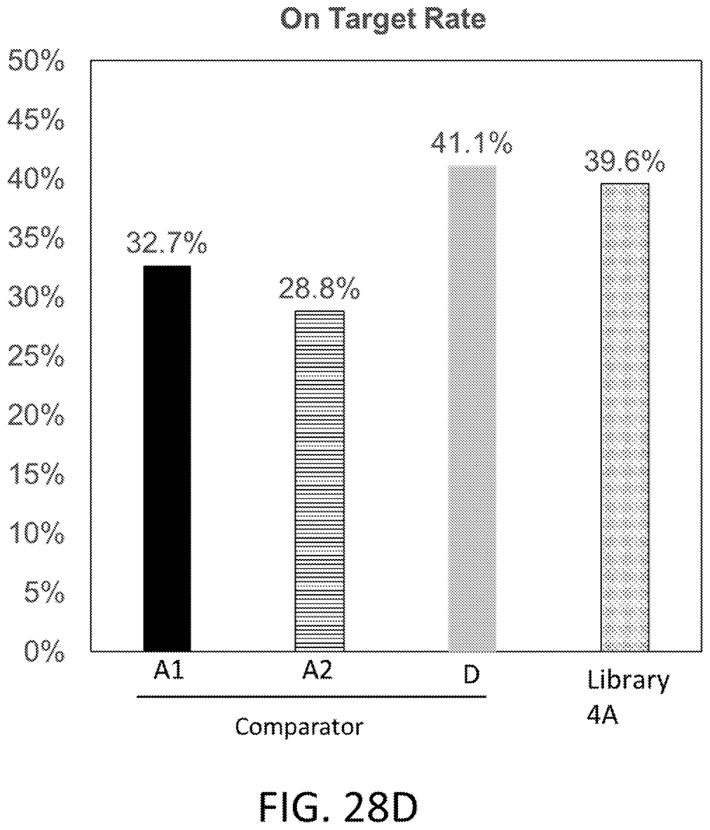

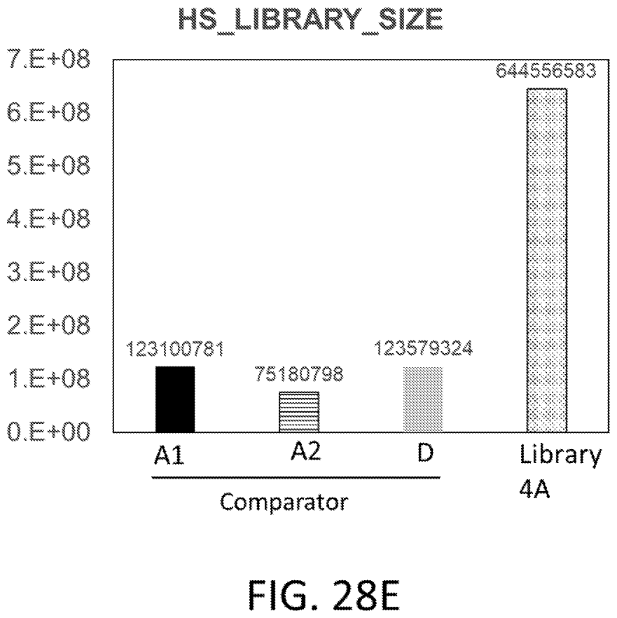

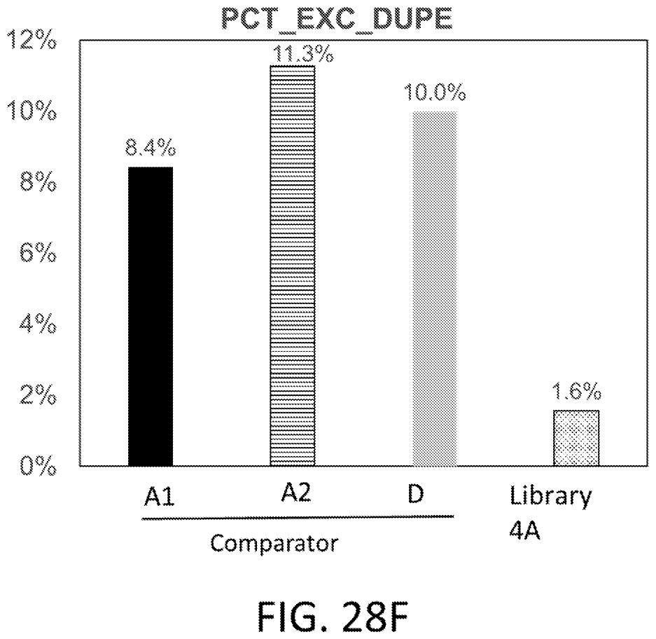

The various libraries were assessed for uniformity, specificity, and duplication rate. As seen in FIG. 28B, Library 4A increased target enrichment efficiency (as measured by fold-80 base penalty) by 35-60% in comparison to comparator kits. As seen in FIGS. 28C-28D, Library 4A had increased specificity and on target rate. On target rate was measured as on target bases divided by PF bases aligned. Library 4A exhibited improved oligonucleotide synthesis, optimized double stranded probes, and compatible buffer and workflow as indicated by duplication rate as seen in FIGS. 28E-28F.

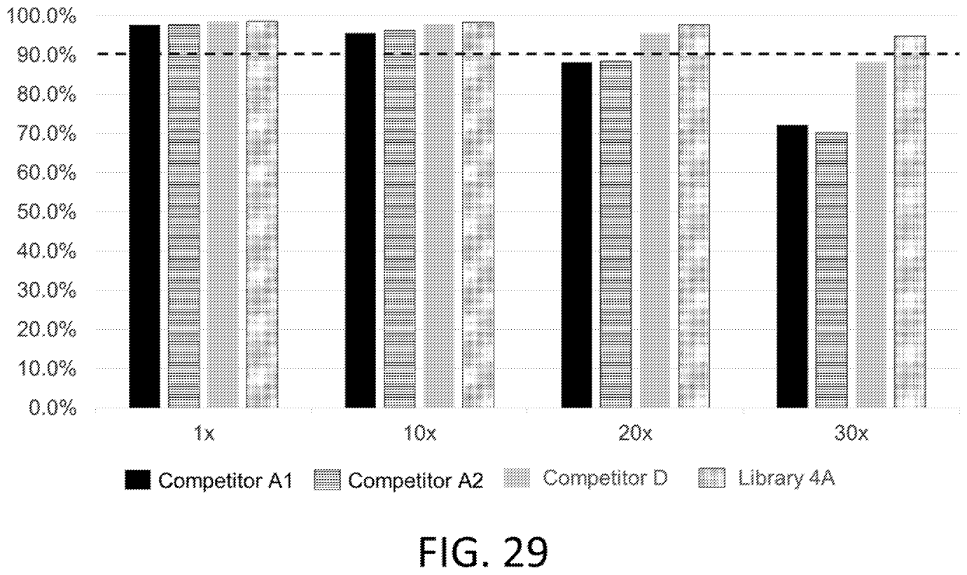

The various libraries were also assessed for depth of coverage and maximized sequencing output. As seen in FIG. 29, 95% targeted bases covered at 30× with 150× total raw sequencing using Library 4A. Table 11 shows that Library 4A maximized sequencing output.

TABLE 11

| | Comparator | Comparator | Comparator | Library |
|---|---|---|---|---|
| | A1 | A2 | D | 4A |
| Capture Size | 38.0 | 35.8 | 38.8 | 33.1 |
| Sequencing | 83,047,756 | 74,745,634 | 54,745212 | 40,398,726 |
| Required | | | | |
| for 90% of | | | | |
| bases at 30x | | | | |
| GigaBases | 8.3 | 7.4 | 5.4 | 4.0 |
| per Exome | | | | |

### Example 16. Flexible and Modular Custom Panels

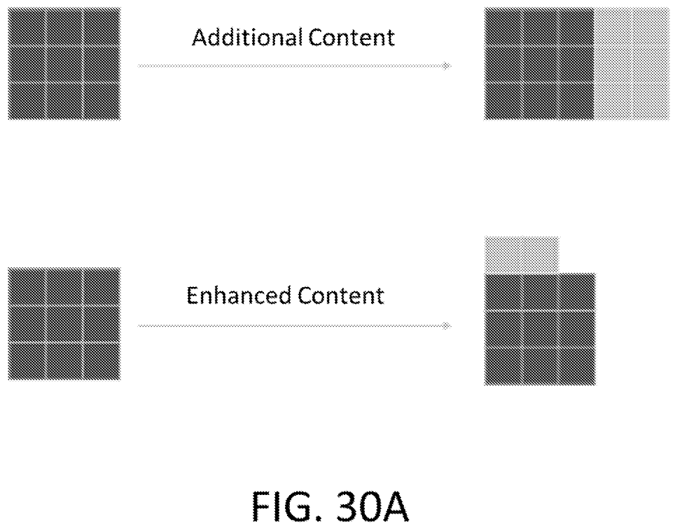

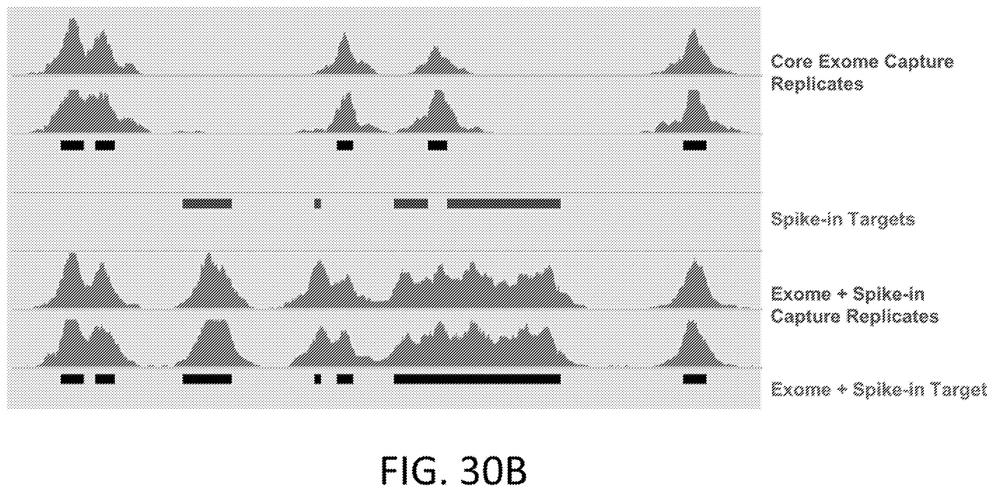

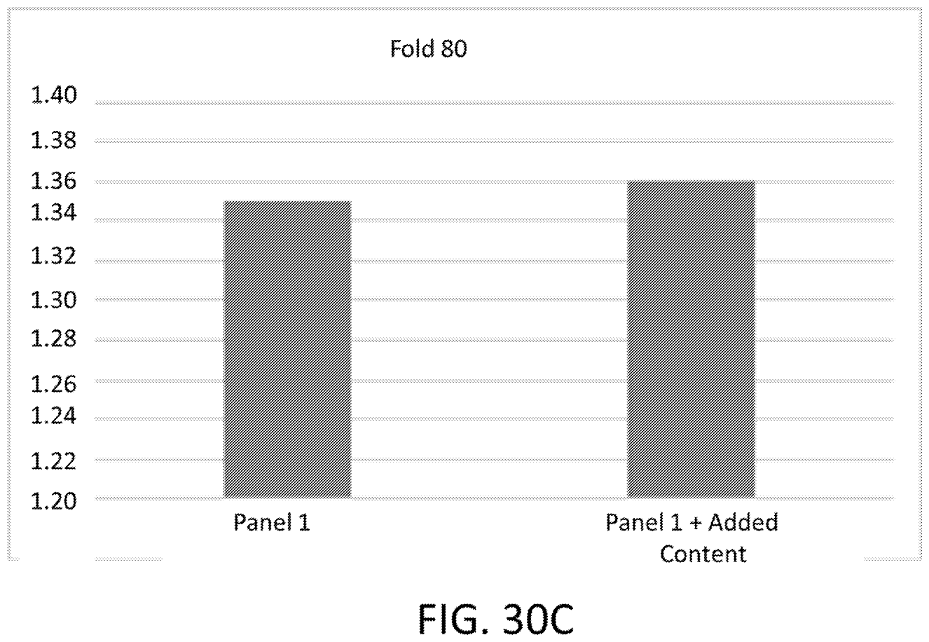

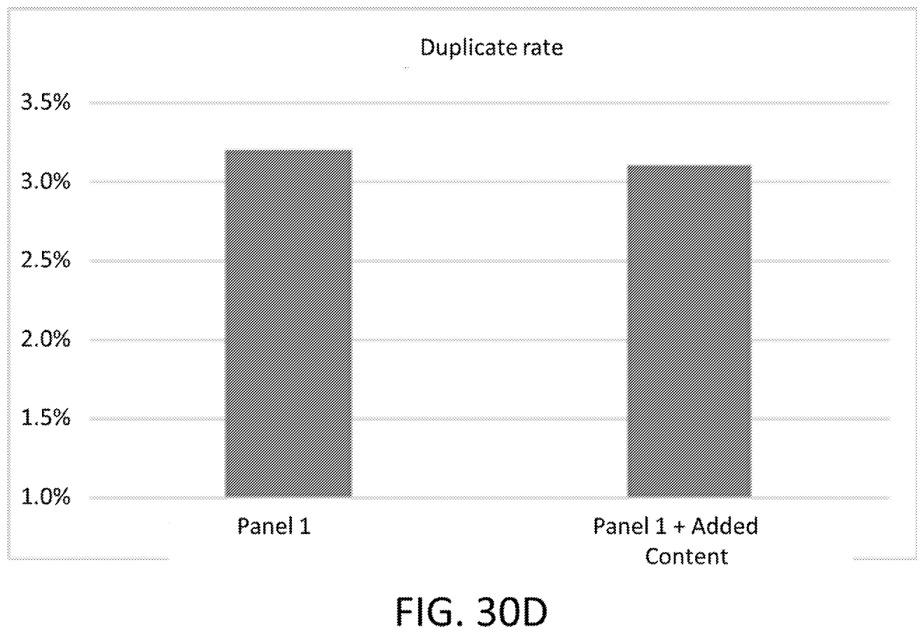

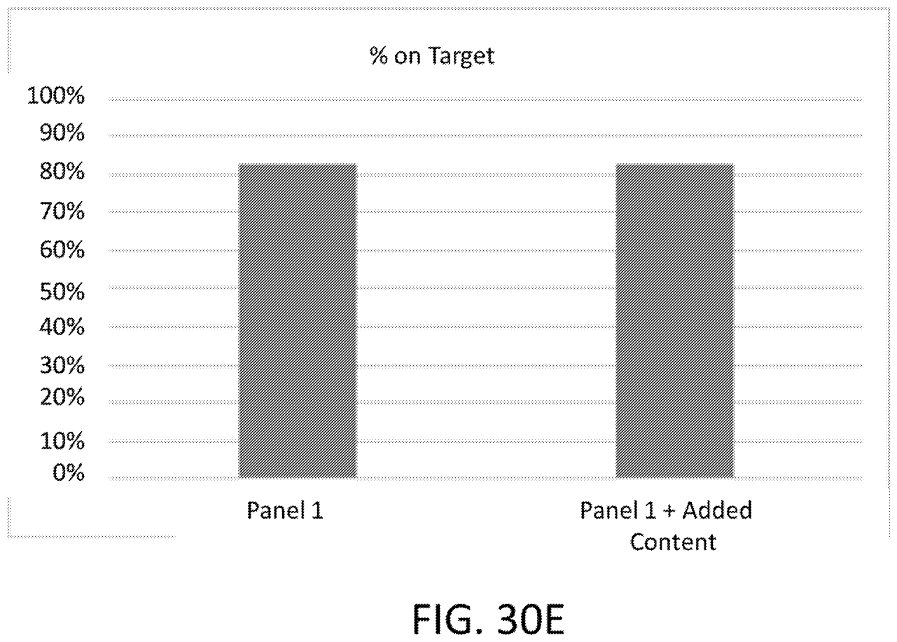

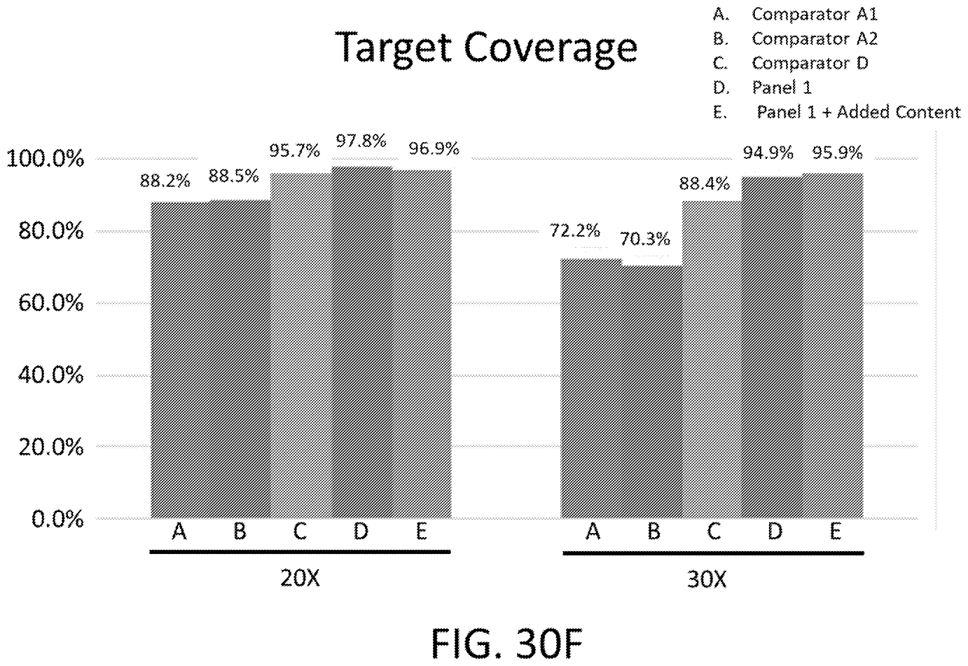

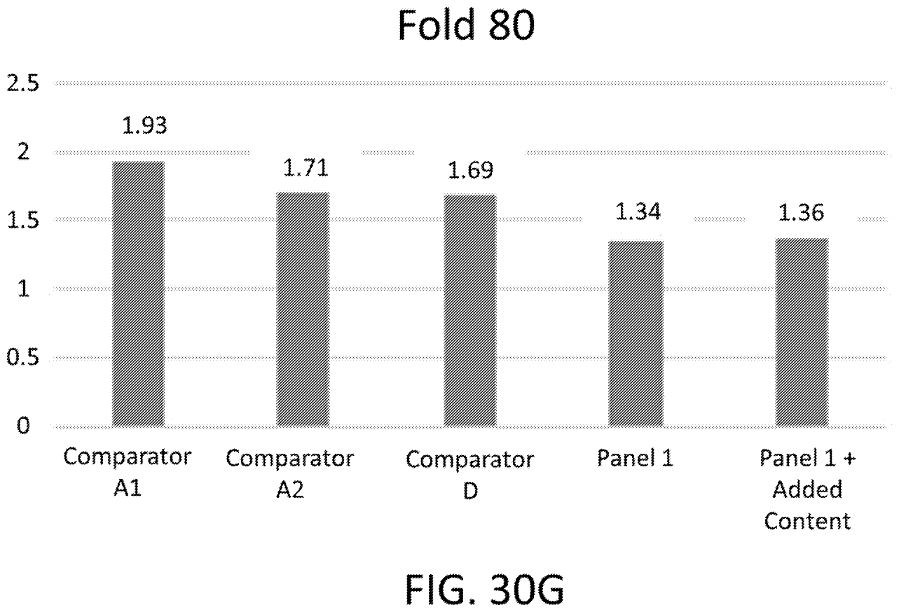

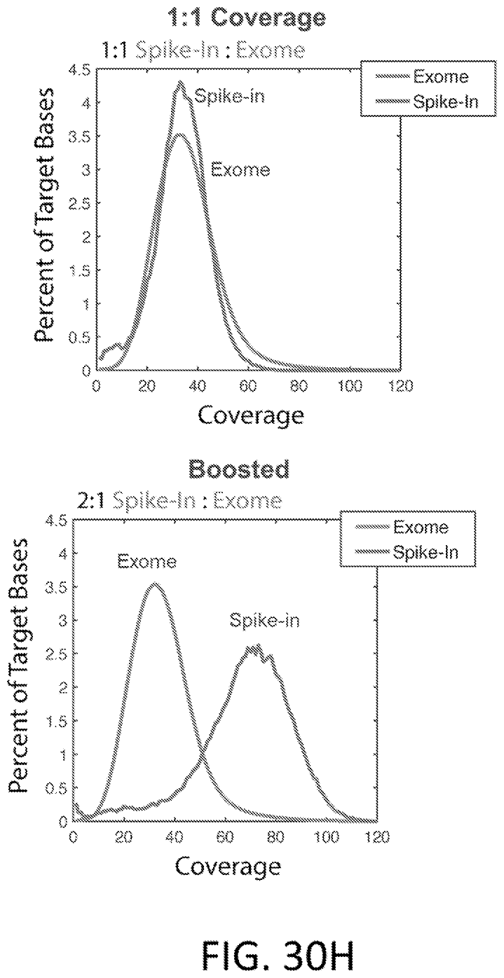

Content can be added to or enhanced. See FIGS. 30A-30B. Adding content to the panel increases the number of targets covered. Enhancing content to the panel refers to the coverage of specific regions.

3 Mb of additional target regions was added derived from the RefSeq database. The production of this panel increased coverage and did not decrease performance. Coverage improved to >99% of the RefSeq, CCDS, and GENCODE databases. Further, the custom panel displayed high uniformity and on-target rate, as well as a low duplicate rate (all results based on 150× sequencing).

The database coverage as seen in Table 12 was increased using the custom panels as described herein. The data compared the overlap between panel content to the protein-coding regions in the databases annotated on the primary human genome assembly (alternative chromosomes were excluded) as of May 2018 (UCSC genome browser). Comparator A1, Comparator A2, and Comparator D are commercially available comparator panels. Comparisons were performed using the BEDtools suite and genome version indicated in parentheses. The addition of 3 Mb of content improved the coverage of RefSeq and GENCODE databases to >99%.

TABLE 12

| Database Coverage | | |
|---|---|---|
| RefSeq | CCDS21 | GENCODE v28 |
| (35.9 Mb) | (33.2 Mb) | (34.8 Mb) |

Experiment 1

| | | | |
|---|---|---|---|
| Panel 1 | 92.3% | 99.5% | 95.1% |
| Panel 1+ Supplemental | 99.2% | 99.5% | 99.1% |
| Probes | | | |
| Comparator A1 (hg19)* | 88.3% | 91.9% | 90.8% |
| Comparator A2 (hg38)* | 91.0% | 94.6% | 94.0% |
| Comparator D (hg19) | 94.1% | 98.3% | 95.7% |

Experiment 2

| | | | |
|---|---|---|---|
| Panel 1 | 91.8% | 99.9% | 99.8% |
| Panel 1 + Supplemental | 99.2% | 99.9% | 99.8% |
| Probes | | | |
| Comparator Al (hg19)* | 91.7% | 92.0% | 90.8% |
| Comparator A2 (hg38)* | 95.4% | 100% | 99.2% |
| Comparator D (hg19) | 98.3% | 99.2% | 95.9% |

FIGS. 30C-30E show data from Panel 1 and Panel 1+Supplemental Probes on Fold (FIG. 30C), duplicate rate (FIG. 30D), and percent on target (FIG. 30E). FIG. 30F and FIG. 30G show comparative data for target coverage (FIG. 30F) and fold-80 base penalty (FIG. 30G).

FIG. 30H shows the tunable target coverage of the libraries described herein. As seen in FIG. 30H in the top panel, there was 34.9 mean coverage and 91% of target bases at greater than 20× were observed. As seen in FIG. 30H in the bottom panel, there was 67.5 mean coverage and 97% of target bases at greater than 20× were observed.

### Example 17. RefSeq Design

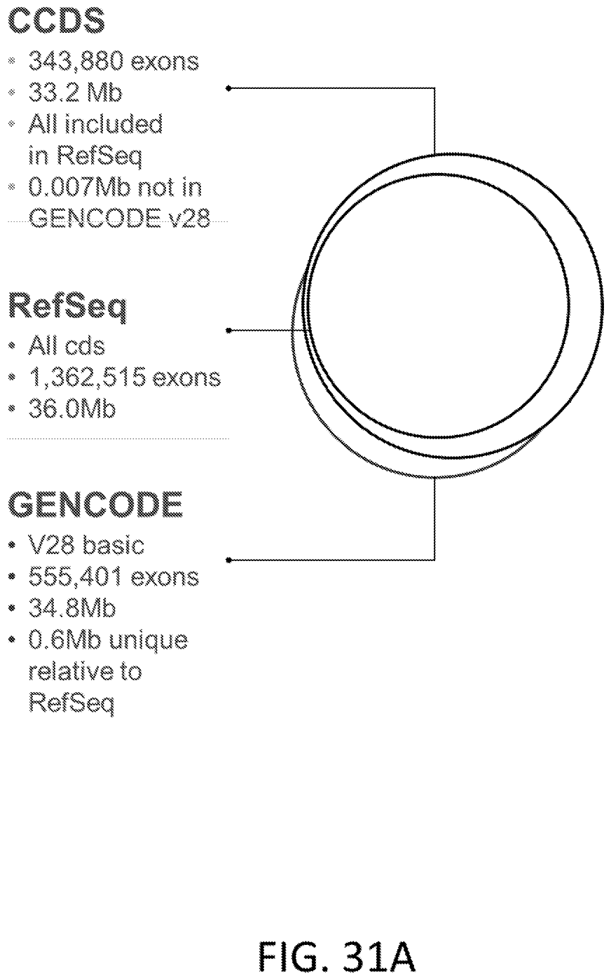

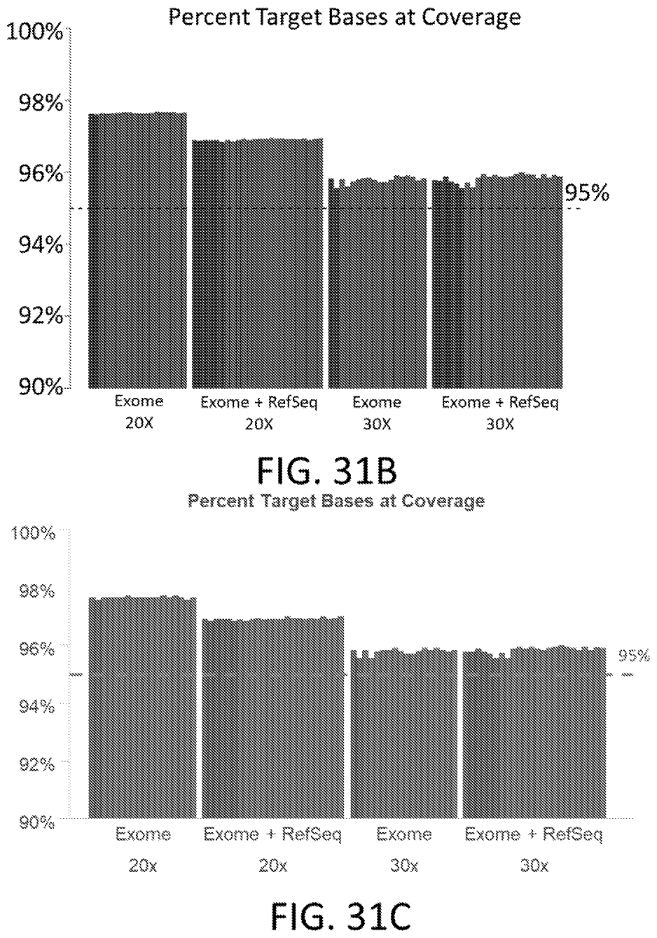

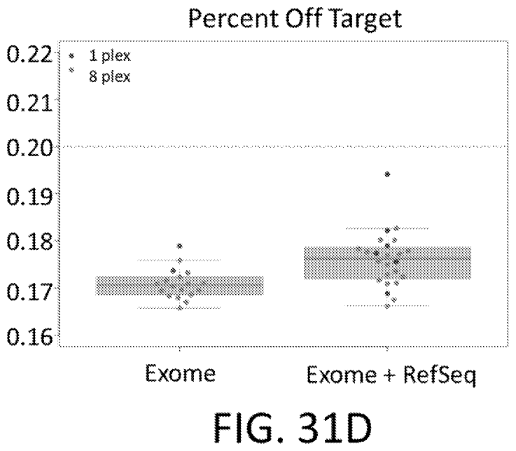

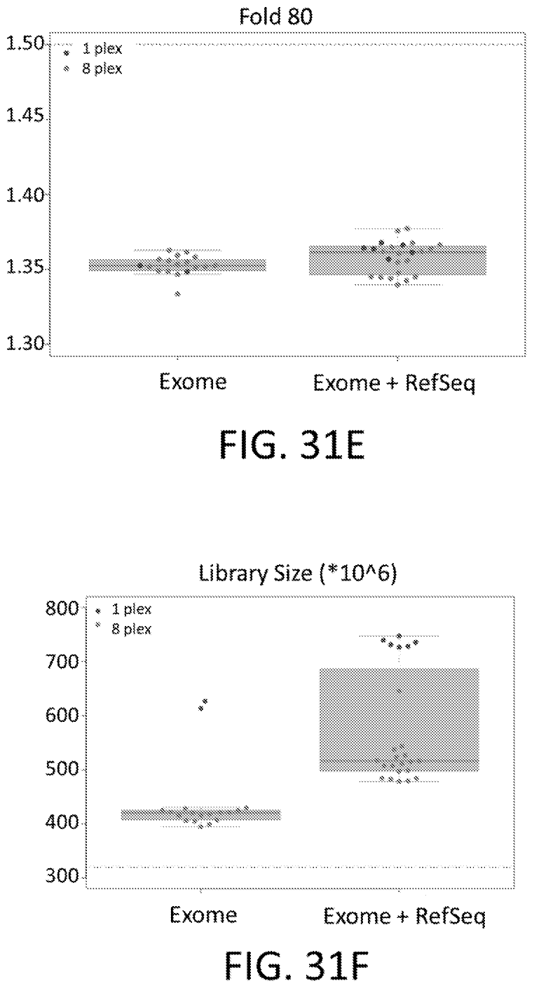

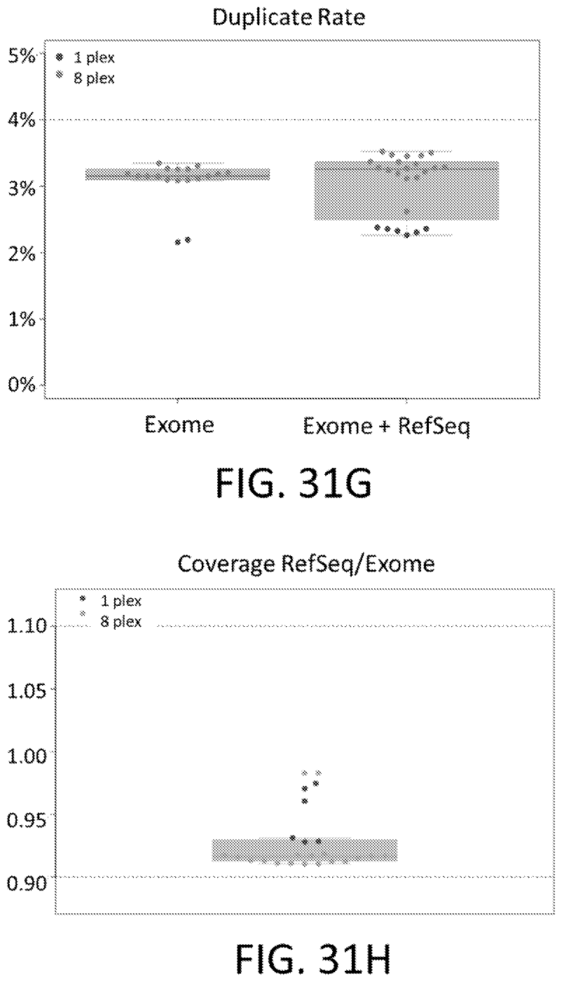

A RefSeq panel design was designed in hg38 and included the union of CCDS21, RefSeq all coding sequence, and GENCODE v28 basic coding sequences. The size of RefSeq alone (Exome) was 3.5 Mb and the combined Core Exome+RefSeq (Exome+RefSeq) was 36.5 Mb. Experiments were run using 50 ng of gDNA (NA12878) as 1-plex and 8-plex run in triplicate, and evaluated at 150× sequencing with 76 bp reads. The target file was 36.5 Mb. See FIG. 31A

The RefSeq panel design was assessed for depth of coverage, specificity, uniformity, library complexity, duplicate rate, and coverage rate. FIGS. 31B-31C shows depth of coverage. More than 95% of target bases at 20× were observed. More than 90% of target bases at 30× were observed. FIG. 31D shows specificity of the RefSeq panel. The percent off target was less than 0.2. FIG. 31E shows uniformity of the RefSeq panel. The fold 80 was less than 1.5. FIG. 31F shows the complexity of the library. The library size was greater than 320 million. FIG. 31G shows the duplicate rate of the RefSeq panel. The duplicate rate was less than 4%. FIG. 31H shows the coverage ratio of the RefSeq panel. The coverage ratio was between 0.9 and 1.1. As seen in FIG. 31H, the coverage ratio was less than 1.1.

### Example 18. Custom Panel Designs Across a Range of Panel Sizes and Target Regions

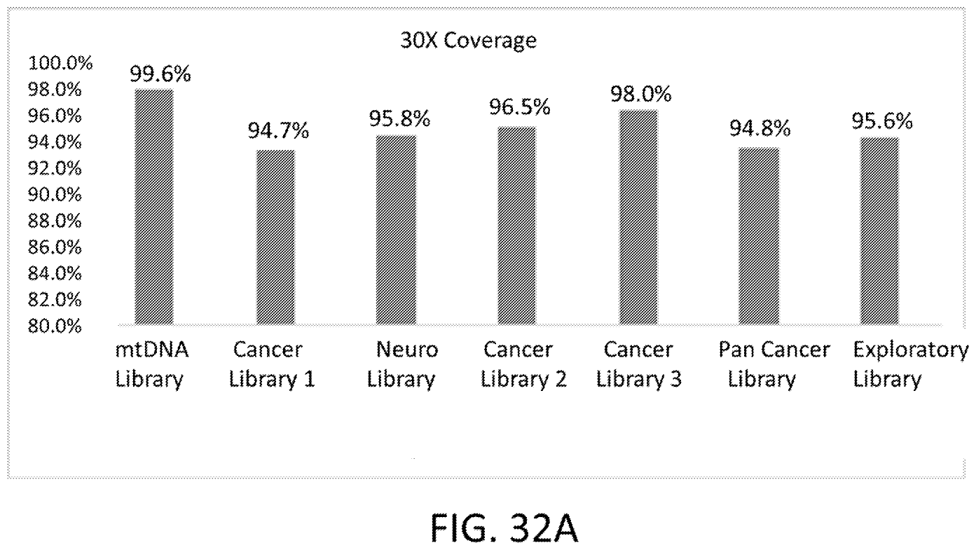

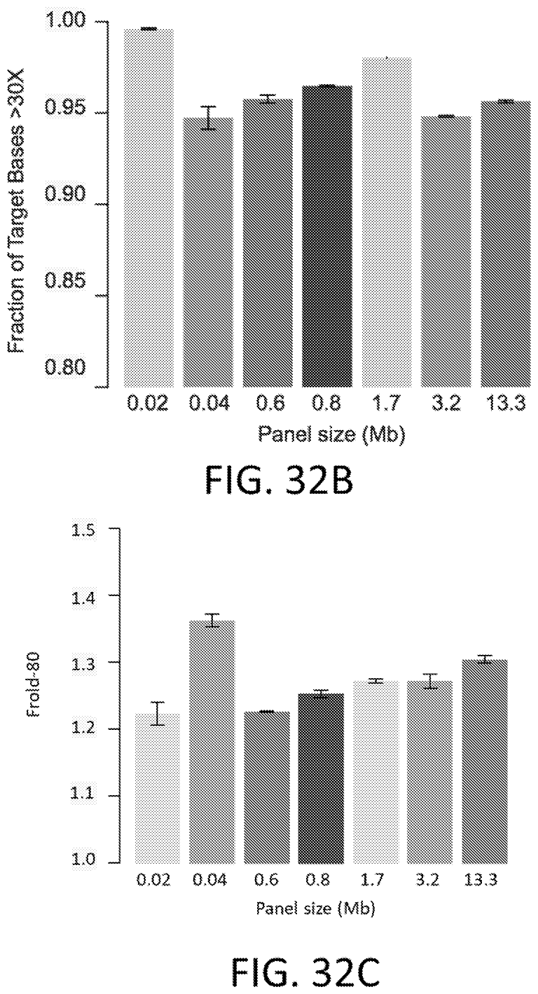

Sequencing data was acquired using the general method of Example 6. Details of the library are seen in Table 13. Briefly, hybrid capture was performed using several target enrichment panels designed herein using 500 ng of gDNA (NA12878; Coriell) per single-plex pool following manufacturer's recommendations. Sequencing was performed with a NextSeq 500/550 High Output v2 kit to generate 2×76 paired end reads. Data was downsampled to 150× of target size and analyzed using Picard Metrics with a mapping quality of 20; N=2. The panels resulted in a high percentage of on-target reads, as well improved uniformity and low duplication rate. FIGS. 32A-32B show percentage of reads in each panel achieving 30× coverage and FIG. 32C shows uniformity (fold-80).

TABLE 13
Panel Description

| | Target | | |
|---|---|---|---|
| | Size | | |
| Name | (Mb) | Probes | Genes |
| mtDNA Library | 0.02 | 139 | 37 |
| Cancer Library | 0.04 | 384 | 50 |
| Neurodegenerative | 0.6 | 6,024 | 118 |
| Library | | | |
| Cancer Library 2 | 0.8 | 7,446 | 127 |
| Cancer Library 3 | 1.7 | 19,661 | 522 |
| Pan-Cancer | 3.2 | 31,002 | 578 |
| Library | | | |
| Exploratory | 13.3 | 135,937 | 5,442 |
| Cancer Library | | | |

### Example 19. Enrichment Workflow

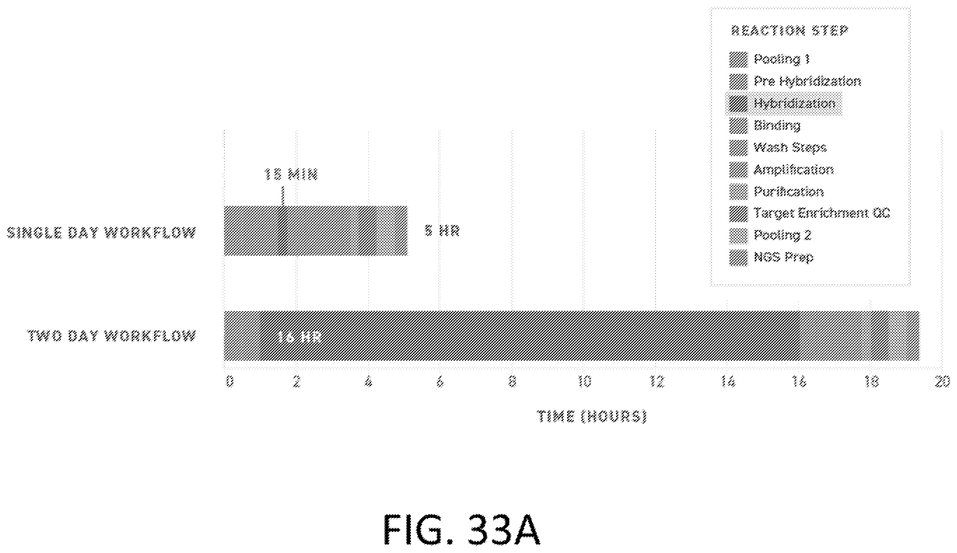

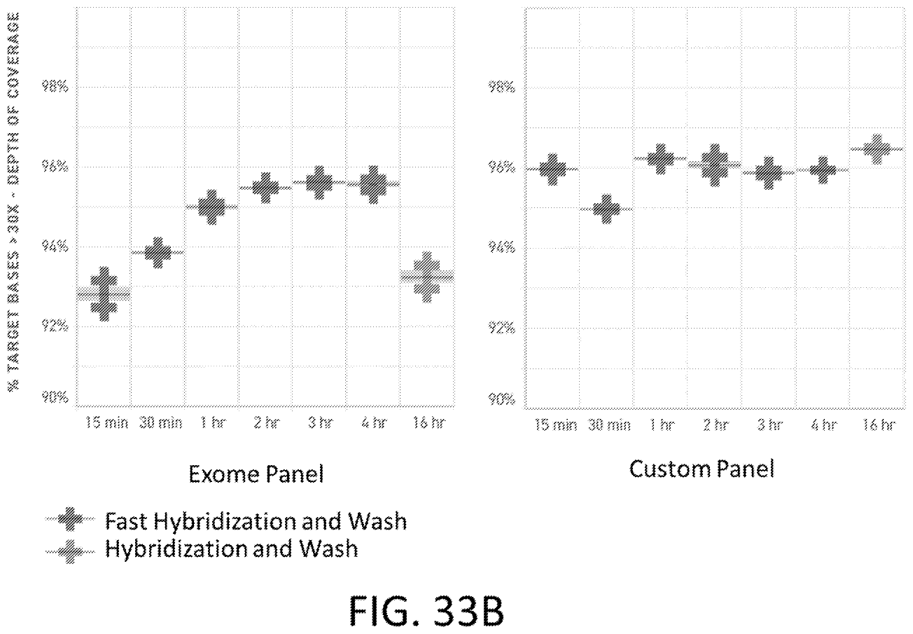

An enrichment workflow timeline is seen in FIG. 33A. Sequencing data was acquired using the general method of Example 6. Briefly, genomic DNA (NA12878, Corriell) was hybridized and captured using either an exome panel or custom panel. A “fast” hybridization buffer was used with liquid polymer during hybridization of the two different probe libraries (exome probes or custom panel) to the nucleic acid sample, and the capture/hybridization reaction was heated at 65° C. for various periods of time in a PCR thermocycler, with a lid temperature of 85° C. Following sequencing, Picard HS_Metric tools (Pct_Target_Bases_30×) with default values were used for sequence analysis. For either panels, a 15-min hybridization in Fast Hybridization Solution produced an equivalent performance to the 16-hr standard hybridization, and increasing hybridization times improved performance over the standard protocol using conventional hybridization buffers as seen in FIG. 33B.

### Example 20. Target Enrichment Using Nanoball Sequencing

Target enrichment panels were sequenced using nanoball sequencing. Briefly, nanoball sequencing uses rolling circle amplification (RCA) to amplify fragments of genomic DNA into DNA nanoballs. The DNA nanoballs are adsorbed onto a flow cell and the fluorescence at each position is determined and used to identify the base.

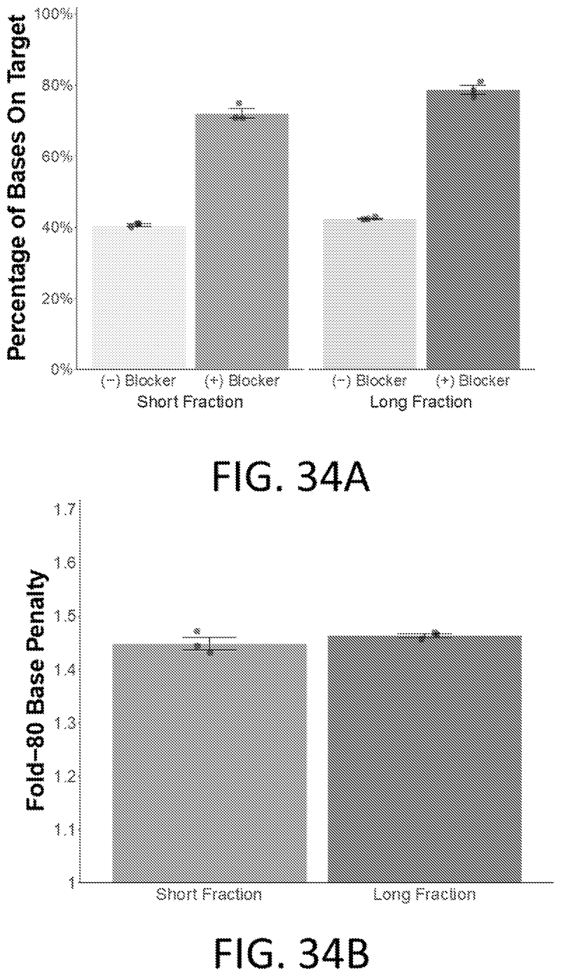

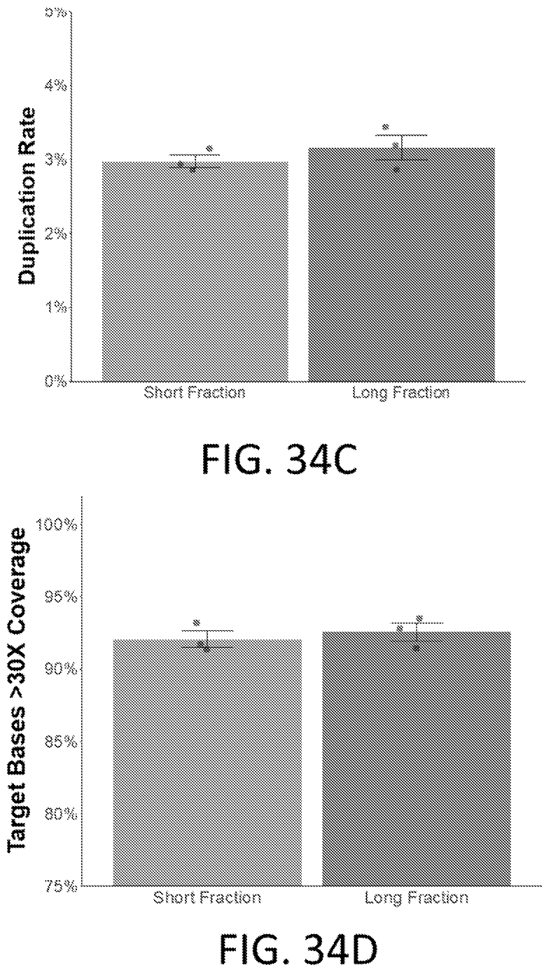

Libraries were prepared with two different insert sizes and sequenced using nanoball sequencing. Circularized adaptors were compatible for nanoball sequencing. The libraries were assessed for on-target rate, specificity, duplication rate, coverage. As seen in FIGS. 34A-34D, there was an increase in percentage of on-target rate from 40% to 75% using the circularized adaptors (FIG. 34A), greater uniformity with a fold 80 at about 1.45 (FIG. 34B), lower duplication rate at about 3% (FIG. 34C), and about 92% target bases at 30× coverage or higher were observed (FIG. 34D).

### Example 21. Blockers which Bind Stem Regions of Adapters

Different commercially available adapter systems comprise different stem (Y-stem, yoke) lengths and melting temperatures (Table 14), such as standard dual barcode adapter system T; transposase adapter system N; and adapter system B designed for nanoball-based sequencing.

TABLE 14
Summary of the Y-stem regions of various adapter systems.

| | Length between | | % of the Y-stem | % GC |
|---|---|---|---|---|
| | genomic material | Length | annealing region length | of the |
| Commercial | and last base | of | (defined from genomic | Y-stem |
| Adapter | before the index | Y-stem | material to the last | annealing |
| System | (bp) | (bp) | base before the barcode) | region |
| T | 33 | 13 | 39.4% | 54% |
| N | 33 | 19 | 57.8% | 37% |
| B (single index) | 32 | 8 | 25.0% | 50% |

Following the general procedures of Example 19, blocking nucleic acids comprising locked nucleic acids (LNAs) were used with an N adapter system during enrichment/capture, and NGS performance as a function of percentage observed “off bait” (fraction of PF_BASES_ALIGNED that are mapped away from any baited region, OFF_BAIT_BASES/PF_BASES_ALIGNED) was measured. Generally, increasing the number of locked nucleic acids annealing to the adapter stem region led to poorer off bait performance (Table 15).

TABLE 15
Observed off bait performance with blockers containing various numbers of
DNA modifications that increase melting temperature in the sequence that is
designed to anneal to the Y-stem of the adapter of an N adapter system.

| Index: | 1 | 2 | 3 | 4 | |
|---|---|---|---|---|---|
| | #of LNAs in Y-stem annealing | | | | Observed Off Bait Performance |
| Experiment | portion of universal blocker | | | | (lower values are desired) |
| 1 | N/A | 4 | 3 | N/A | 30 ± 5% |
| 2 | N/A | 8 | 8 | N/A | 53 ± 10% |
| 3 | N/A | 7 | 6 | 0 | 40 ± 5% |
| 4 | N/A | 0 (10)* | 0 (9)* | 0 | Not tested |

*Number in parentheses indicates number of LNAs outside of Y-stem annealing portion.

Without being bound by theory, decreased performance in some instances may be caused by an increase in undesirable hybridization species populations B-D (FIGS. 36B-36D), and a decrease in the desired species population A (FIG. 36A), Table 16.

TABLE 16
Overview of quantity of DNA modifications that increase melting
temperature in Y-stem annealing region of blocker and the expected
off-bait performance during target enrichment workflows.

| Quantity of DNA | | | | | Expected |
|---|---|---|---|---|---|
| modifications that | | | | | Off-Bait |
| increase melting | | | | | Performance |
| temperature in | | | | | During |
| Y-stem annealing | Population | Population | Population | Population | Target |
| region of blocker | A | B | C | D | Enrichment |
| + | ++++ | + | + | ++ | ++++ |
| ++ | +++ | ++ | ++ | ++ | +++ |
| +++ | + | ++ | ++ | + | ++ |
| ++++ | + | ++++ | ++++ | + | + |

### Example 22. PUSH-PULL Universal Blockers

Universal blockers may be designed with regions that both enhance and decrease binding affinity of targeted sequences to cause an overall net positive increase in affinity and improvement off-bait performance during target enrichment. Such designs provide potential advantages, for example: 1) each region can be either theoretically or empirically tuned for a given desired level of off-bait activity during target enrichment applications; 2) each region can be altered with either a single type of chemical modification or multiple types that may either increase or decrease overall affinity of a molecule for a targeted sequence; 3) the melting temperature of all individual members of a blocker set must be held above a specified temperature for optimal performance with other modifications (e.g., LNA &BNA); 4) a given set of blockers will improve off bait performance independent of index length, independent of index sequence, and independent of how many adapter indices are present in hybridization.

One approach of addressing the Y-stem adapter annealing portion of Universal Blockers is by completely removing DNA alterations and designing blockers with only the standard A, C, G, & T bases in this problematic region. There is also the possibility of adding in additional DNA modifications that will decrease binding affinity for a given region. If this is accompanied by a region where DNA alterations are introduced to increase binding affinity, then one can create blocker oligos designed with both increased and decreased regions of affinity for a given target region. An example of a commercially available modification that can be introduced during chemical synthesis is 2′-deoxylnosine.

While some designs utilize stretches of these types of moieties (6-10 bp in length) to cover adapter barcodes, they could also be utilized in a sparse fashion across a sequence to decrease melting temperature (Tₘ). A random 18 bp sequence is shown below without and with the inclusion of different numbers of 2′-deoxyinosine moieties to demonstrate that the Tₘ can be adjusted to a desired target (Table 17). When such sequences are concatenated with sequences containing moieties that increase Tₘ, one can generate hybrid molecules with varying thermodynamic properties. In such hybrid molecules, specific regions can be thermodynamically tuned to specific melting temperatures to either avoid or increase the affinity for a particular targeted sequence. This combination of modifications is designed to help increase the affinity of the blocker molecule for specific and unique adapter sequence and decrease the affinity of the blocker molecule for repeated adapter sequence (e.g., Y-stem annealing portion of adapter). Without being bound by theory, such designs may increase binding for desired populations and decrease binding for undesired populations in the context of hybridization during a target enrichment workflow.

An example where the number of moieties that increase affinity in the unique region is held constant and the number of moieties that decrease affinity in the region that binds to the Y-stem portion of the adapter are increased is presented Table 17.

TABLE 17
Melting temperature effects on a
random sequence when 2′-deoxyinosine
moieties are introduced.

| Sequence | Number of | Tₘ (° C.; calculated at |
|---|---|---|
| (5′ to 3′; ‘/ideoxyI/’ | 2′- | https://geneglobe.qiagen.com/ |
| denotes a single | deoxyInosine | us/explore/tools/ |
| 2′-deoxyInosine moiety). | moieties | tm-prediction/form) |
| ACTACGTACGATCGATCG | 0 | 59 |
| (SEQ ID NO: 7) | | |
| ACTA/ideoxyI/GTACGATC/ | 2 | 52 |
| ideoxyI/ATCG | | |
| (SEQ ID NO: 8) | | |
| ACTA/ideoxyI/GTA/ | 4 | 42 |
| ideoxyI/GATC/ideoxyI/AT/ | | |
| ideoxyI/G (SEQ ID NO: 9) | | |

When the number of DNA modifications that decrease affinity in the Y-stem annealing region of the blocker are increased, the populations ‘A’& ‘D’ dominate and either have the desired (A, FIG. 36A) or minimal effect (D, FIG. 36D) (Table 18). As the number of DNA modifications that decrease affinity in the Y-stem annealing region of the blocker are decreased, the populations ‘B’ & ‘C’ dominate and have undesired effects where daisy-chaining or annealing to other adapters can occur (‘B’ FIG. 36B) or sequester blockers where they are unable to function properly (C, FIG. 36C).

TABLE 18
Overview of quantity of DNA modifications that increase melting temperature in Y-stem
annealing region of blocker and the expected off-bait performance during target
enrichment workflows. Population A corresponds to FIG. 36A, Population B
corresponds to FIG. 36B, Population C corresponds to FIG. 36C,
and Population D corresponds to FIG. 36D.

| | Quantity of | | | | | |
|---|---|---|---|---|---|---|
| | DNA | | | | | |
| Quantity of DNA | modifications | | | | | Expected |
| modifications that | that increase | | | | | Off-Bait |
| reduce affinity in | affinity in | | | | | Performance |
| Y-stem annealing | unique adapter | | | | | During |
| region of blocker | region of blocker | Population | Population | Population | Population | Target |
| (varied) | (held constant) | A | B | C | D | Enrichment |
| ++++ | ++ | ++++ | + | + | ++ | ++++ |
| +++ | ++ | +++ | ++ | ++ | ++ | +++ |
| ++ | ++ | + | ++ | ++ | + | ++ |
| + | ++ | + | ++++ | ++++ | + | + |

### Example 23. Universal Adapters Covering Indices with Universal Bases

The index on both single or dual index adapter designs are either partially or fully covered by universal blockers that have been extended with specifically designed DNA modifications to cover adapter index bases. Such designs provide potential advantages, such as 1) adjustments to either partially or fully cover barcodes of various lengths from either side of the index; 2) the melting temperature of all individual members of a blocker set in some instances is held above a specified temperature for optimal performance with other modifications (e.g., LNAs and/or BNAs); and 3) a given set of blockers will improve off bait performance when index length is equal to or greater than a defined minimal length, independent of sequence, and independent of how many adapter indices are present in hybridization.

Blockers are designed in such a manner that they bind to regions which are not part of the adapter index (FIG. 37A). As a consequence, all index bases with this design are left completely exposed (i.e., ‘1|2|3| . . . |(n−1)|n’ in FIG. 37A). This design is also extended with a variety of moieties that will extend blockers to cover index bases. Covering index bases in such a manner is demonstrated to enhance off-bait performance during target enrichment when an individual index of a dual index system is covered from a single side by either 3 bp or 5 bp stretches of 2′-deoxyinosine moieties (FIG. 37B). Additional designs include FIGS. 37C-37G.

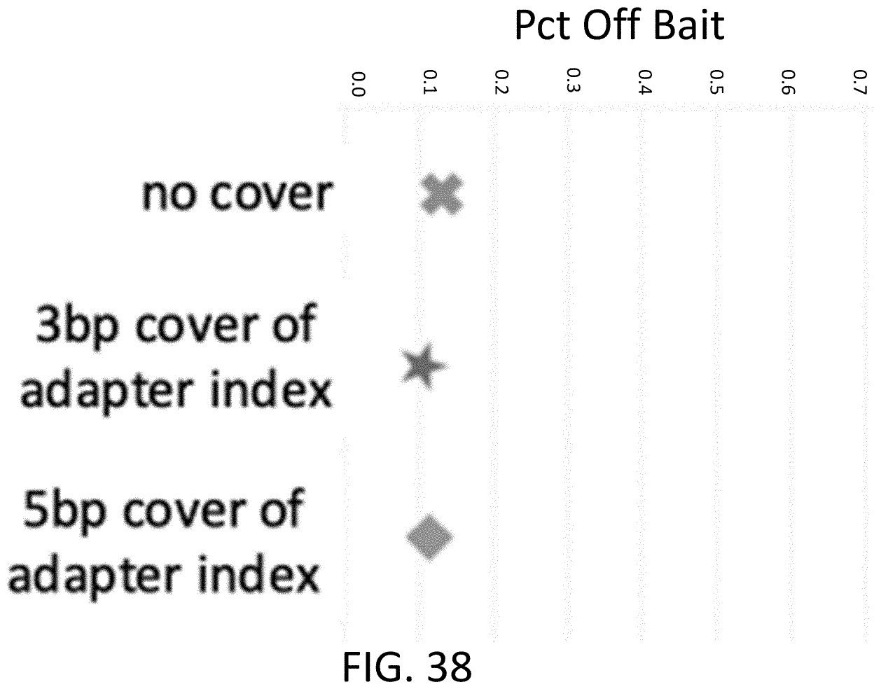

Following the general procedures of Example 19, a 33.1 Mb exome panel was used for capture with a two hour hybridization time, and NGS metrics were obtained. Improvements were observed for (a) percent off bait (PCT_OFF_BAIT), (b) uniformity (FOLD_80_BASE_PENALTY), and (c) depth of coverage (PCT_TARGET_BASES_30) (FIG. 38, Table 19). Such changes can have a significant impact on the number of samples that can be placed onto next generation sequencing machines (e.g., Illumina's NGS NovaSeq platform).

TABLE 19
Summary of metrics of blocker sets that cover various number of index bases.

| Universal Blocker Design | PCT_OFF_BAIT | FOLD_80_BASE_PENALTY | PCT_TARGET_BASES_30X |
|---|---|---|---|
| no cover of 10 bp index | 0.128385 | 1.476669 | 0.926015 |
| 3 bp cover of 10 bp adapter | 0.105497 | 1.447092 | 0.93253 |
| index | | | |
| 5 bp cover of 10 bp adapter | 0.112926 | 1.459129 | 0.931812 |
| index | | | |

### Example 24. Exome Enrichment for Targeted Methylation Sequencing

Materials and Methods. Genomic DNA samples from NA12878 (Coriell Institute) and EpiScope® hypo- and hypermethylated gDNA controls (<5% and >95% methylated HCT116 DKO gDNA, respectively) were mechanically sheared to a size of −300 bp (on Covaris® ME220). Samples of various simulated methylation levels were prepared by blending sheared hypo- and hypermethylated controls. 500 ng of gDNA input were put into the Swift Accel-NGS® Methyl-seq DNA Library Kit in combination with bisulfite treatment (Zymo EZ DNA Methylation-Lightning Kit), Omega Bio-Tek Mag-Bind RxnPure Plus SPRI Beads, and KAPA HiFi Uracil+ DNA Polymerase. 200 ng of gDNA input were put into the NEBNext® Enzymatic Methyl-seq Kit. Sheared samples and libraries were verified with the Agilent BioAnalyzer 7500 and the Invitrogen Qubit Broad Range Kits.

Following the general protocol of Example 19, fast hybridization buffer was used for a four hour hybridization with four methylation panels covering a range of different target sizes (0.05, 1.0, 1.5, and 3.0 Mb). 200 ng of library was used for each single-plex capture, followed by 2×151 bp sequencing on an Illumina NextSeq 550 with a v2.5 High Output Kit. Alignment and methylation analyses were performed using Bismark 19.1 and Picard HsMetrics after sampling to a raw coverage of 250× per sample.

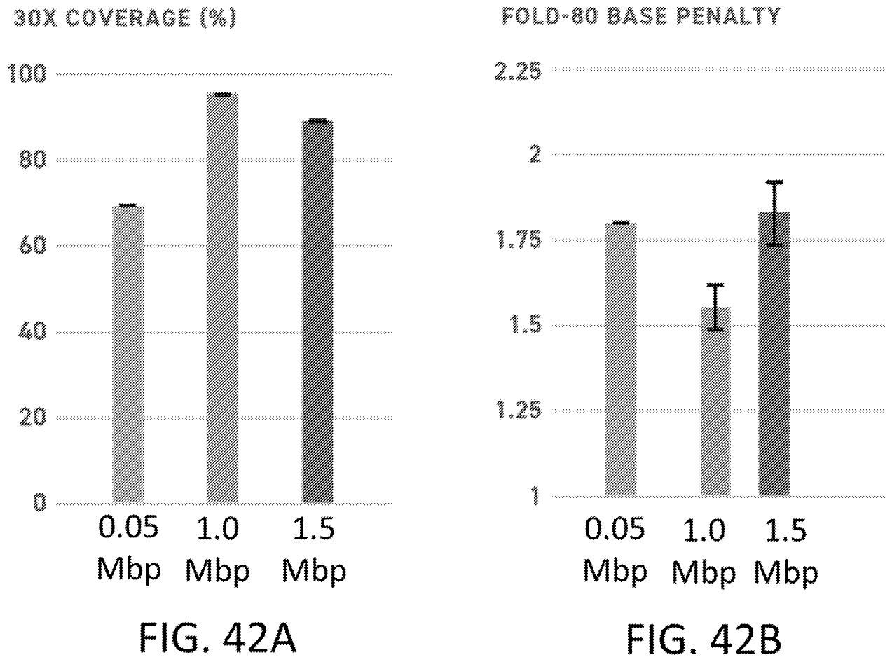

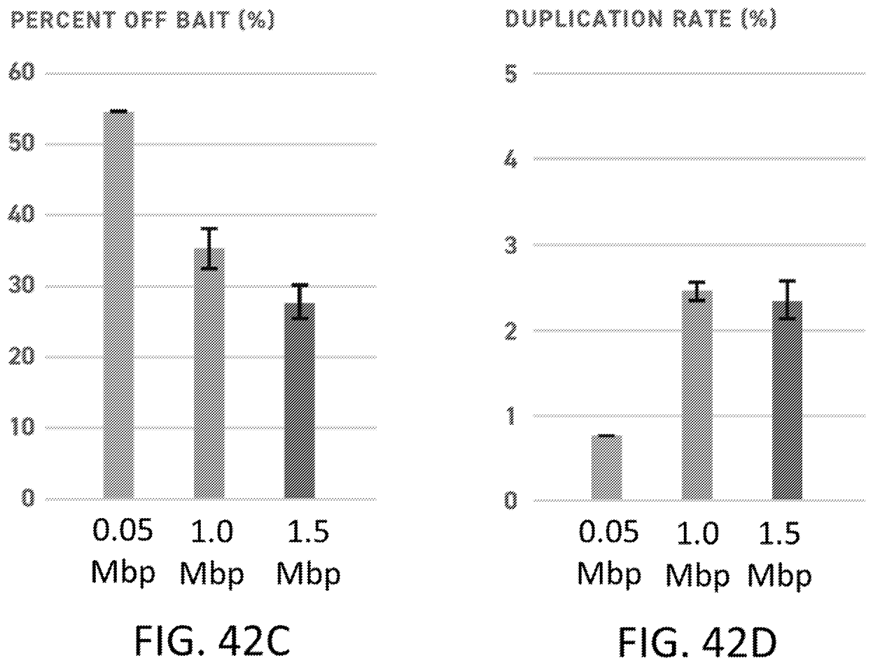

Results. While pre-capture conversion can enable highly sensitive epigenetic applications, key challenges originate from the reduced complexity of the genome after conversion. Compared to non-methylated panels, this generally leads to markedly high off-target (levels >50-60%), lower sequencing coverage of baits, and a strong reduction of capture uniformity (fold 80 base penalty values >2.5). Results obtained from three of the panels covering a wide range of different methylation targets are shown in FIGS. 42A-42D. Panels evaluated showed off-target values as low as 27%. The 0.05 Mb panel showed higher off-target compared to the other three panels. Without being bound by theory, his may be due to the nature of an extremely small target size. Capture uniformity was >2.5 fold 80 and reached values as low as 1.75 and 1.5. The duplication rate was very low among all four tested panels, indicating the capture step was efficient and able to retain high sample complexity throughout the workflow. Overall, with 250× raw sequencing coverage, a raw coverage of bases higher than 84% at 20× and 70% at 30×, even for the smallest panel, was achieved.

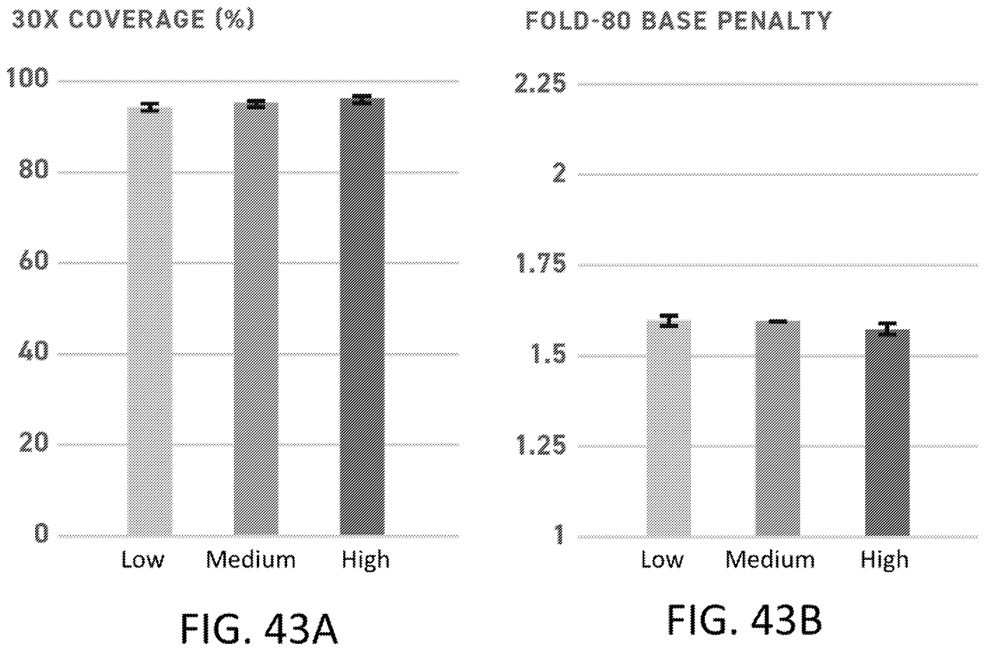

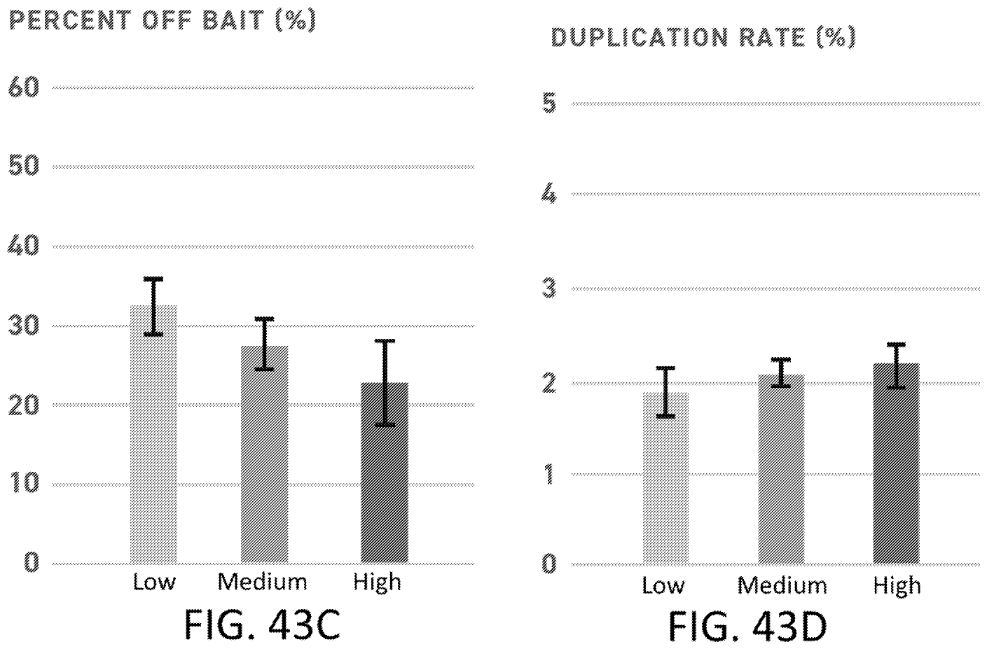

Adaptive panel design optimization algorithms enable the use of empirical data from capture experiments to learn about specific probe characteristics to quantitatively tune performance. This method becomes particularly useful for methylation panels where controlling high off-target rates becomes a priority. In addition, using data collected for over ˜30,000 methylation targets, informative sequence features were derived and used develop optimized default panel designs with three levels of stringency. The 1 Mb panel was used as an example of default panels with low, medium, and high stringency which provide increasing control of off-target rates while leading to only minor changes in other key metrics (FIGS. 43A-43D).

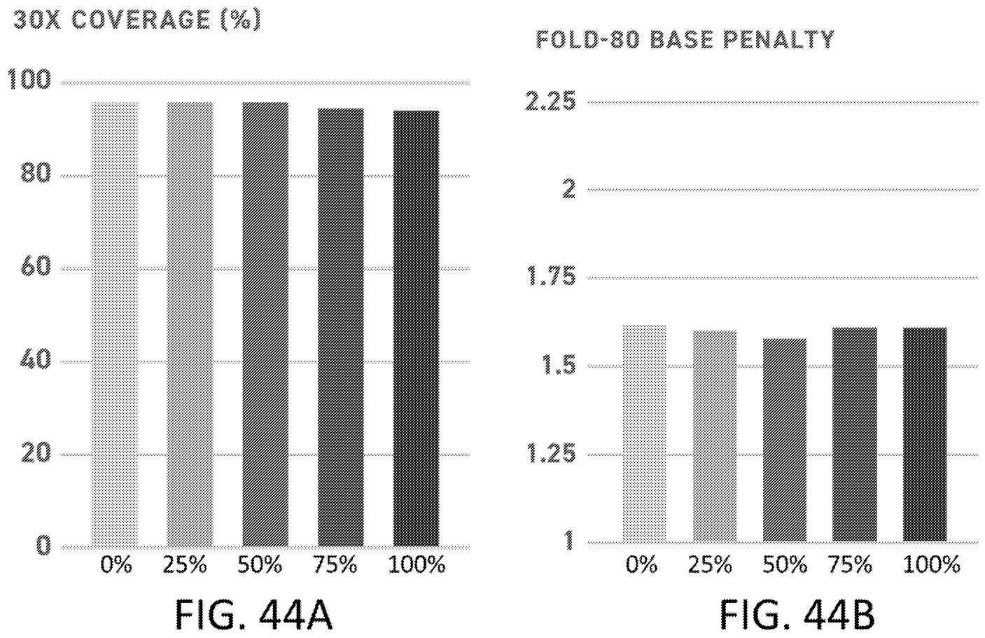

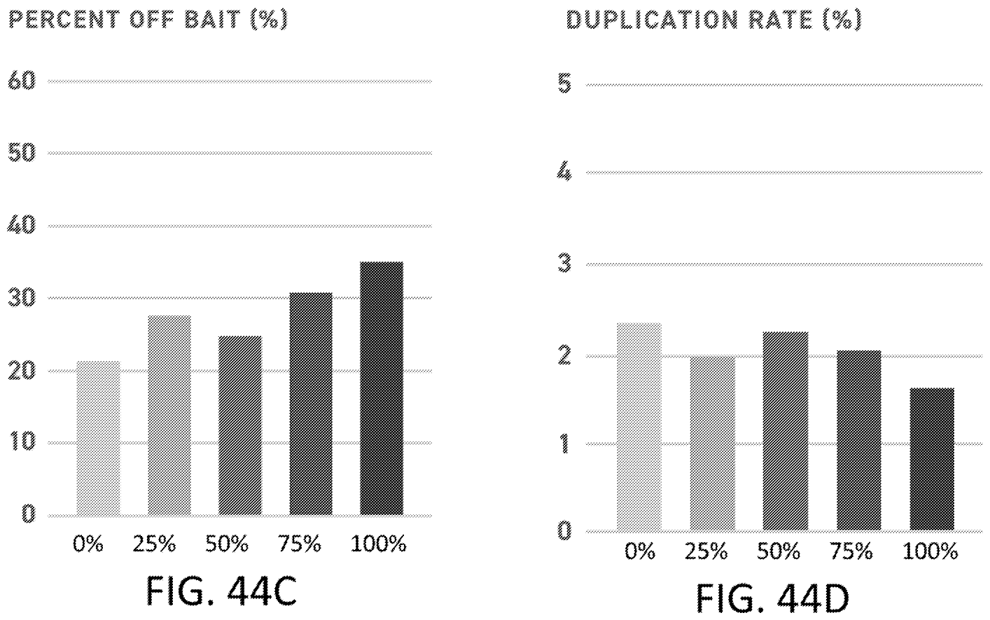

To evaluate compatibility across a range of possible methylation levels, captures on the medium stringency 1 Mb panel were performed with gDNA libraries generated from hypomethylated and hypermethylated cell lines blended to final ratios of 0, 25, 50, 75, and 100% methylation, respectively. FIGS. 44A-44D highlight key capture metrics with the bar showing average values and standard errors representing the variability in capture performance between differentially methylated samples. Metrics show little to no response to varying methylation levels, demonstrating the compatibility of the system with a wide range of methylation states including hypo- and hypermethylated DNA.

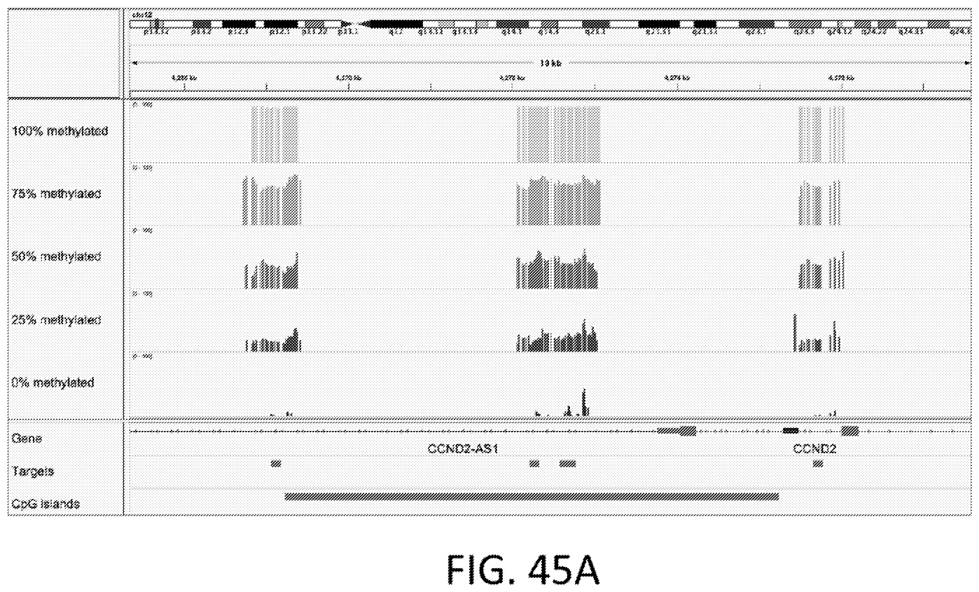

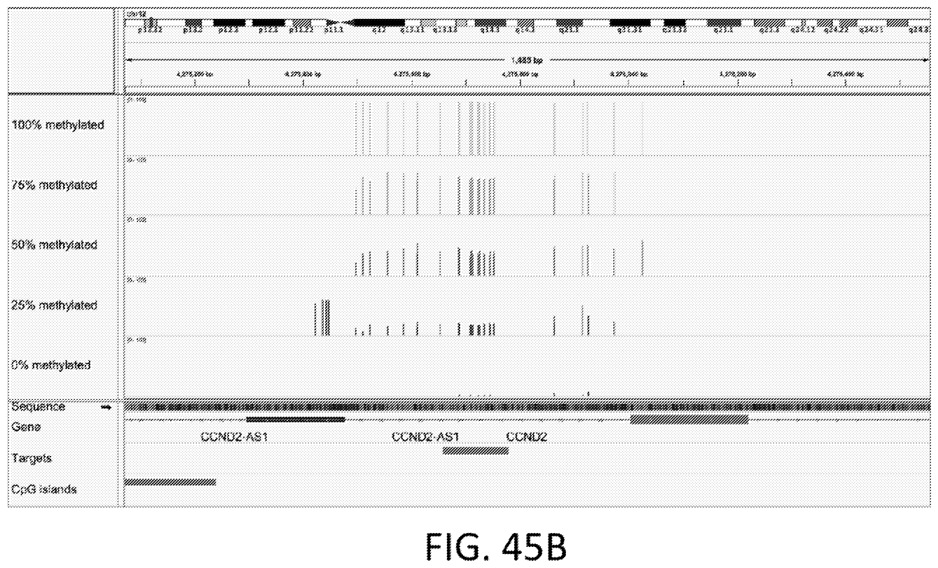

Changes in methylation levels of promoters and other regulatory elements are emerging as some of the most sensitive markers available for the early detection of cancer. Targeted methylation sequencing can detect and quantify differential levels of DNA methylation. Hypo- and hypermethylated DNA were blended to different ratios and used for capture with a 1 Mb panel. FIGS. 45A and 45B highlight the detection of different DNA methylation levels along targets and individual CpG sites in the clinically relevant Cyclin D2 locus, which is known to change methylation states in certain cancers (e.g., breast cancer). Detecting methylated cytosines involves the conversion of unmethylated cytosines to thymine while methylated cytosines are protected from conversion. Traditionally conversion occurred through a chemical bisulfite method. Other methods including enzymatic conversion of unmethylated cytosines have been adopted in the field at increasing rates. Each conversion method has advantages and disadvantages, such as greater potential sensitivity of the enzyme to conversion reaction conditions or the context biased degradation of DNA by bisulfite.

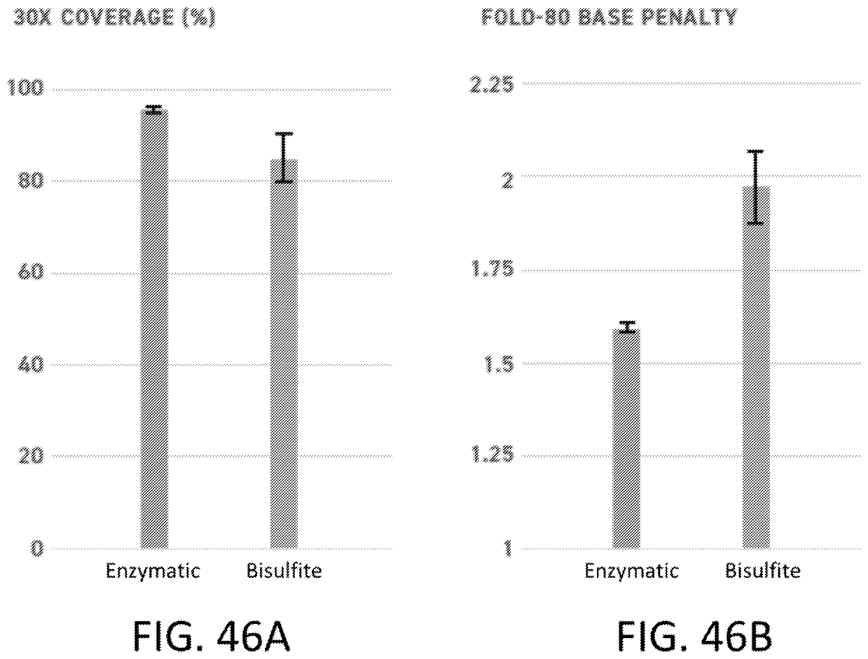

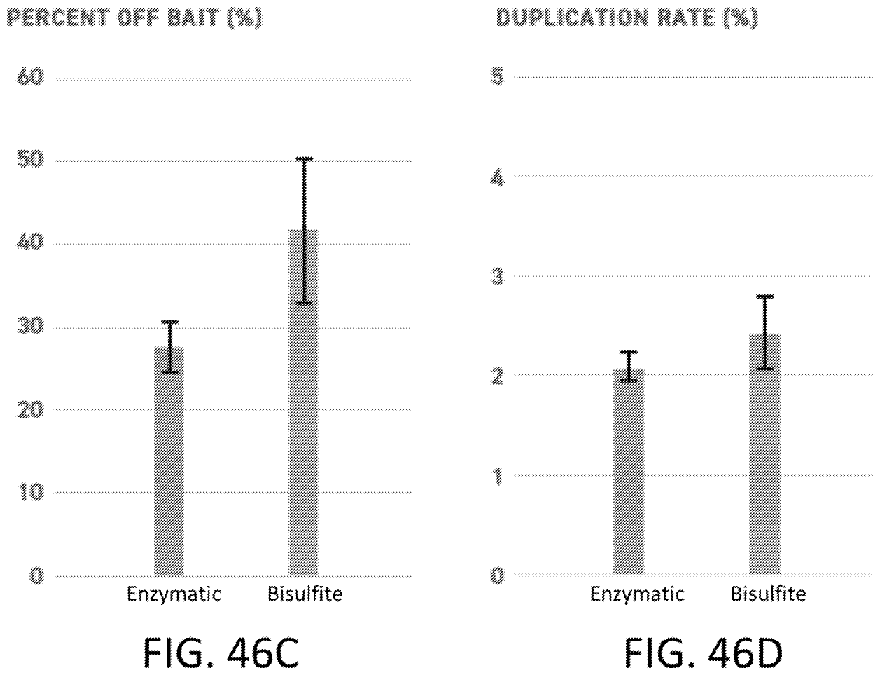

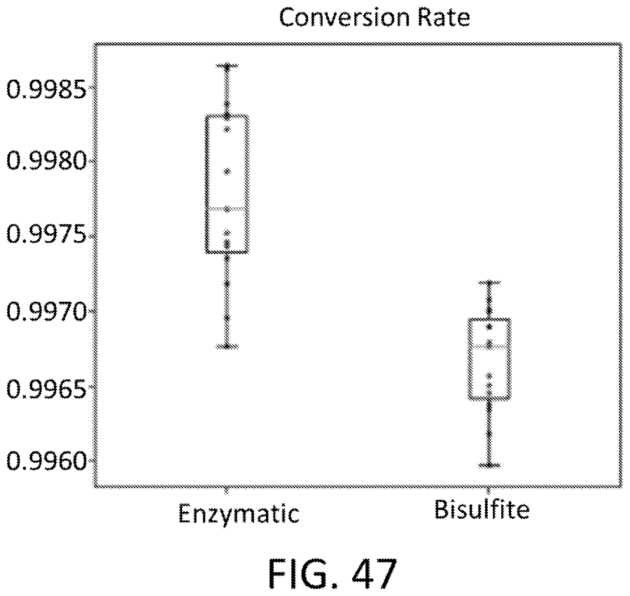

Methylation sequencing with the panels synthesis herein were compatible with both enzymatic and bisulfite based approaches (FIGS. 46A-46D). Conversion rates, measured as the fraction of cytosines converted in non-CpG sites were >99.5% for both methods (FIG. 47). Overall capture metrics were comparably on the same order for both library preparation methods, though certain metrics such as uniformity, and off-target were reduced for the bisulfite method. Without being bound by theory, the reduced uniformity may be at least partially due to the inherent GC bias introduced by the bisulfite based library preparation method (data not shown).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-35. (canceled)

36. A composition comprising:
at least three polynucleotide blockers, wherein the at least three polynucleotide blockers are configured to bind to one or more regions of an adapter-ligated sample nucleic acid, wherein the adapter-ligated sample nucleic acid comprises:
i) a first non-complementary region, a first index region, a second non-complementary region, and a first yoke region; and
ii) a third non-complementary region, a second index region, a fourth non-complementary region, and a second yoke region;
wherein the first yoke region and the second yoke region are complementary, and wherein the first non-complementary region and the second non-complementary region are not complementary; and
iii) a genomic insert, located adjacent to the first yoke region and the second yoke region, wherein at least one polynucleotide blockers is not complementary to the first yoke region or the second yoke region, and comprises at least one nucleotide analog configured to increase the binding between the polynucleotide blocker and the adapter-ligated sample nucleic acid.

37. The composition of claim 36, wherein the composition wherein at least two polynucleotide blockers are not complementary to the first yoke region or the second yoke region, and each comprises at least one modified nucleobase configured to increase the binding between the polynucleotide blocker and the adapter-ligated sample nucleic acid.

38. The composition of claim 36, wherein at least one index region comprises a barcode or unique molecular identifier.

39. The composition of claim 36, wherein at least one index region is 5-15 bases in length.

40. The composition of claim 36, wherein at least one of the polynucleotide blockers comprises at least one universal base.

41. The composition of claim 40, wherein the at least one universal base is 5-nitroindole or 2-deoxyinosine.

42. The composition of claim 40, wherein the at least one universal base is configured to overlap with at least one index sequence.

43. The composition of claim 40, wherein at least two universal bases are configured to overlap with at least two index sequences.

44. The composition of claim 40, wherein at least two of the polynucleotide blockers comprise at least one universal base, wherein each of the at least one universal base overlaps with at least one index sequence.

45. The composition of claim 42, wherein the overlap is 2-10 bases in length.

46. The composition of claim 36, wherein the composition comprises no more than four polynucleotide blockers.

47. The composition of claim 36, wherein the polynucleotide blocker comprises one or more locked nucleic acids (LNAs) or one or more bridged nucleic acids (BNAs).

48. The composition of claim 36, wherein the polynucleotide blocker comprises at least 5 nucleotide analogues.

49. (canceled)

50. The composition of claim 36, wherein the polynucleotide blocker has a Tm of at least 78 degrees C.

51-53. (canceled)

54. A method for nucleic acid hybridization comprising:
providing an adapter-ligated sample nucleic acid library comprising a plurality of genomic inserts;
contacting the adapter-ligated sample nucleic acid library with a probe library comprising at least 5000 polynucleotide probes in the presence of the composition of claim 36; and
hybridizing at least some of the probes to the genomic inserts.

55. The method of claim 54, wherein the sample nucleic acid library comprises at least 1 million unique genomic inserts.

56. The method of claim 54, wherein at least some of the genomic inserts comprise human DNA.

57. The method of claim 54, wherein the method further comprises generating an enriched sample nucleic acid library.

58. The method of claim 57, wherein the method further comprises sequencing the enriched sample nucleic acid library.

59. The method of claim 54, wherein the sample nucleic acid library comprises adapters configured for next generation sequencing.


SEQUENCE LISTING (9 sequences)

SEQ ID NO: 1 - DNA, 62 bp, Artificial Sequence
  Description of Artificial Sequence Synthetic oligonucleotide
  modified_base (51)..(52): Thymidine-succinyl hexamide CED phosphoramidite
agacaatcaaccatttggggtggacagccttgacctctagacttcggcattttttttttttt

SEQ ID NO: 2 - DNA, 112 bp, Artificial Sequence
  Description of Artificial Sequence Synthetic polynucleotide
  modified_base (101)..(102): Thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, Artificial Sequence
  Description of Artificial Sequence Synthetic primer
atgcggggttctcatcatc

SEQ ID NO: 4 - DNA, 20 bp, Artificial Sequence
  Description of Artificial Sequence Synthetic primer
cgggatccttatcgtcatcg

SEQ ID NO: 5 - DNA, 20 bp, Artificial Sequence
  Description of Artificial Sequence Synthetic primer
aatgatacggcgaccaccga

SEQ ID NO: 6 - DNA, 24 bp, Artificial Sequence
  Description of Artificial Sequence Synthetic primer
caagcagaagacggcatacgagat

SEQ ID NO: 7 - DNA, 18 bp, Artificial Sequence
  Description of Artificial Sequence Synthetic oligonucleotide
actacgtacgatcgatcg

SEQ ID NO: 8 - DNA, 18 bp, Artificial Sequence
  Description of Artificial Sequence Synthetic oligonucleotide
  modified_base (5)..(5): 2′-deoxyInosine
  modified_base (14)..(14): 2′-deoxyInosine
actangtacgatcnatcg

SEQ ID NO: 9 - DNA, 18 bp, Artificial Sequence
  Description of Artificial Sequence Synthetic oligonucleotide
  modified_base (5)..(5): 2′-deoxyInosine
  modified_base (9)..(9): 2′-deoxyInosine
  modified_base (14)..(14): 2′-deoxyInosine
  modified_base (17)..(17): 2′-deoxyInosine
actangtangatcnatng